(19) United States
(12) Patent Application Publication
(10) Pub. No.: US 2014/0093537 A1
(43) Pub. Date: Apr. 3, 2014

(54) IMMUNOGENIC COMPOSITIONS COMPRISING NANOEMULSION AND METHODS OF ADMINISTERING THE SAME

(71) Applicants: James R. Baker, JR., Ann Arbor, MI (US); Douglas Smith, Ann Arbor, MI (US); Ali I. Fattom, Ann Arbor, MI (US); Jakub Simon, Ann Arbor, MI (US)

(72) Inventors: James R. Baker, JR., Ann Arbor, MI (US); Douglas Smith, Ann Arbor, MI (US); Ali I. Fattom, Ann Arbor, MI (US); Jakub Simon, Ann Arbor, MI (US)

(73) Assignees: NANOBIO CORPORATION, Ann Arbor, MI (US); THE REGENTS OF THE UNIVERSITY OF MICHIGAN, Ann Arbor, MI (US)

(21) Appl. No.: 14/042,128

(22) Filed: Sep. 30, 2013

Related U.S. Application Data
(60) Provisional application No. 61/708,008, filed on Sep. 30, 2012.

(51) Int. Cl. A61K 39/245; A61K 39/02; A61K 39/145; A61K 39/155; A61K 39/00; A61K 39/12

(52) U.S. Cl. CPC A61K 39/245; A61K 39/00; A61K 39/12; A61K 39/145; A61K 39/155; A61K 39/02; A61K 39/0011
USPC 424/209.1; 424/184.1; 424/204.1; 424/211.1; 424/231.1; 424/234.1; 424/277.1

(57) ABSTRACT

The present invention provides methods and compositions for the stimulation of immune responses. In particular, the present invention provides immunogenic nanoemulsion compositions and methods of administering the same (e.g., via a heterologous prime/boost protocol (e.g., utilizing the same nanoemulsion in each the prime and boost administrations)) to induce immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

This application claims the benefit of U.S. Pat. Appl. Ser. No. 61/708,008 filed 30 Sep. 2012, which is incorporated herein by reference.

### STATEMENT REGARDING FEDERALLY SPONSORED RESEARCH OR DEVELOPMENT

This invention was made with government support under AI090031 awarded by the National Institutes of Health. The government has certain rights in the invention.

### FIELD OF THE INVENTION

The present invention provides methods and compositions for the stimulation of immune responses. In particular, the present invention provides immunogenic nanoemulsion compositions and methods of administering the same (e.g., via a heterologous prime/boost protocol (e.g., utilizing the same nanoemulsion in each of the prime and boost administrations)) to induce immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

### BACKGROUND

The body's immune system activates a variety of mechanisms for attacking pathogens (See, e.g., Janeway, Jr, C A. and Travers P., eds., in Immunobiology, “The Immune System in Health and Disease,” Second Edition, Current Biology Ltd., London, Great Britain (1996)). However, not all of these mechanisms are necessarily activated after immunization. Protective immunity induced by immunization is dependent upon the capacity of an immunogenic composition to elicit the appropriate immune response to resist or eliminate the pathogen. Depending on the pathogen, cell-mediated and/or humoral immune responses are important for pathogen neutralization and/or elimination.

Many antigens are poorly immunogenic or non-immunogenic when administered by themselves. Strong adaptive immune responses to antigens generally require that the antigens be administered together with an adjuvant, a substance that enhances the immune response (See, e.g., Audbert, F. M. and Lise, L. D. 1993 Immunology Today, 14: 281-284).

The need for effective immunization procedures is particularly acute with respect to infectious organisms that cause acute infections at, or gain entrance to the body through, the gastrointestinal, pulmonary, nasopharyngeal or genitourinary surfaces. These areas are bathed in mucus, which contains immunoglobulins comprising secretory immunoglobulin IgA (See, e.g., Hanson, L. A., 1961 Intl. Arch. Allergy Appl. Immunol., 18, 241-267; Tomasi T. B., and Zigelbaum, S., 1963 J. Clin. Invest., 42, 1552-1560; Tomasi, T. B., et al., 1965 J. Exptl. Med., 121, 101-124). This immunoglobulin is derived from large numbers of IgA-producing plasma cells, which infiltrate the lamina propria regions underlying the mucosal membranes (See, e.g., Brandtzaeg, P., and Baklein, K, 1976 Scand. J. Gastroenterol., 11 (Suppl. 36), 1-45; and Brandtzaeg, P., 1984 “Immune Functions of Human Nasal Mucosa and Tonsils in Health and Disease”, page 28 et seq. in Immunology of the Lung and Upper Respiratory Tract, Bienenstock, J., ed., McGraw-Hill, New York, N.Y.). The secretory immunoglobulin IgA is specifically transported to the luminal surface through the action of the secretory component (See, e.g., Solari, R, and Kraehenbuhl, J-P, 1985 Immunol. Today, 6, 17-20).

Parenteral immunization regimens are usually ineffective in inducing secretory IgA responses. Secretory immunity is most often achieved through the direct immunization of mucosally associated lymphoid tissues. Following their induction at one mucosal site, the precursors of IgA-producing plasma cells extravasate and disseminate to diverse mucosal tissues where final differentiation to high-rate IgA synthesis occurs (See, e.g., Crabbe, P. A., et al., 1969 J. Exptl. Med., 130, 723-744; Bazin, H., et al., 1970 J. Immunol., 105, 1049-1051; Craig, S. W., and Cebra, J. J., 1971 J. Exptl. Med., 134, 188-200).

### SUMMARY OF THE INVENTION

The present invention provides methods and compositions for the stimulation of immune responses. In particular, the present invention provides immunogenic nanoemulsion compositions and methods of administering the same (e.g., via a heterologous prime/boost protocol (e.g., utilizing the same nanoemulsion in each of the prime and boost administrations)) to induce immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

In one embodiment, the invention provides a method of inducing an immune response in a subject (e.g., an immunogen-specific immune response) comprising providing a subject; and an immunogenic composition comprising a nanoemulsion and immunogen; and administering multiple deliveries (e.g., via a prime/boost protocol) of the immunogenic composition to the subject in order to generate a desired immune response in the subject (e.g., an immunogen-specific immune response). In such immunization protocols, a priming delivery may be via a different route of administration than one or more boost deliveries. In preferred embodiments, one or more of the prime and boost deliveries comprises delivering to the subject via a mucosal route (e.g., intranasal, vaginal) an immunogenic composition of the invention. In other preferred embodiments, one or more of the prime and boost deliveries comprises delivering to the subject via a parenteral route (e.g., infusion, injection or implantation) an immunogenic composition of the invention. The invention is not limited by the injectable route of administration. Indeed, any type of injection may be utilized including, but not limited to, subcutaneous, intramuscular, intraperitoneal, intradermal, and/or intravenous administration. In some preferred embodiments, intramuscular injection is utilized. In some embodiments, a prime administration is via a mucosal route (e.g., nasal mucosa, genital mucosa, oral mucosa, rectal mucosa) and a boost administration is via an intramuscular route. For example, in some preferred embodiments, a prime administration is via an intranasal route and a boost administration is via an intramuscular route (e.g., in order to generate an immunogen-specific, T helper type 17 (Th17) immune response. In some embodiments, the same immunogenic composition is used for both the prime and subsequent boost administrations/deliveries. In a preferred embodiment, the same nanoemulsion is used for both the prime and subsequent boost administrations/deliveries. In some embodiments, the same nanoemulsion is used for both the prime and subsequent boost administrations/deliveries, but at a different dilution (e.g., an immunogenic composition comprising the same amount of immunogen and same nanoemulsion is used for both prime and boost administrations, but the percent of nanoemulsion present in the prime administration is different from the percent of nanoemulsion present in the boost administration). In some embodiments, a different nanoemulsion is used for the prime administration than is used in a subsequent boost administration/delivery. In some embodiments, an immunogenic composition comprising the same amount of immunogen and same nanoemulsion is used for both prime and boost administrations. In some embodiments, the amount of immunogen administered to a subject via the immunogenic composition is the same for both prime and boost administrations/deliveries. In some embodiments, the amount of immunogen administered to a subject via the immunogenic composition is different between the prime and boost administrations/deliveries. In a preferred embodiment, the amount of immunogen/antigen delivered in a prime and/or boost administration is an effective amount to induce a desired immune response in a subject. The invention is not limited by the amount of immunogen/antigen delivered in a prime and/or boost administration. Indeed, any amount of immunogen/antigen may be delivered (e.g., independently or together with one or more different immunogens/antigens and/or adjuvants) to a subject including, but not limited to, those amounts disclosed herein. In some embodiments, a first amount of immunogen is utilized in a prime administration/delivery, and a different, second amount of immunogen is utilized in a boost administration/delivery (e.g., in order to generate a desired type and/or strength of immune response). The invention is not limited by the type of immunogens/antigens delievered via a method of the invention. Indeed, a variety of immunogens/antigens may be administered including, but not limited to, those disclosed herein. In a preferred embodiment, the antigen is a respiratory syncytial virus (RSV) antigen. In accordance with an aspect of the present invention, there is provided an immunogenic composition for eliciting an immune response (e.g., a desired type (e.g., Th1, Th2, Th17, etc.) or strength (e.g., certain immunogen-specific antibody titer)) in a subject, the immunogenic composition comprising a nanoemulsion adjuvant described herein. The invention is not limited by the type of nanoemulsion utilized in an immunogenic composition administered. Indeed, any nanoemulsion may be utilized including, but not limited to, those disclosed herein.

For example, in one aspect of the invention, there is provided a method of generating an immune response in a subject comprising administering thereto an immunogenic nanoemulsion composition of the present invention (e.g., independently and/or in combination with one or more antigenic (e.g., microbial pathogen (e.g., bacteria, viruses, etc.) protein, glycoprotein, lipoprotein, peptide, glycopeptide, lipopeptide, toxoid, carbohydrate, tumor-specific antigen))) components. In some embodiments, a host immune response attained via administration of a nanoemulsion adjuvant to a host subject is a humoral immune response. In some embodiments, a host immune response attained via administration of a nanoemulsion adjuvant to a host subject is a cell-mediated immune response. In some embodiments, a host immune response attained via administration of a nanoemulsion adjuvant to a host subject is an innate immune response. In some embodiments, a host immune response attained via administration of a nanoemulsion adjuvant to a host subject is a combination of innate, cell-mediated and/or humoral immune responses. In some embodiments, a composition comprising a nanoemulsion adjuvant further comprises a pharmaceutically acceptable carrier.

In some embodiments, the prime and one or more boost deliveries of an immunogen/antigen utilizes an immunogenic composition comprising a nanoemulsion and immunogen/antigen. In other embodiments, the prime and one or more boost deliveries of an immunogen/antigen utilizes an immunogenic composition comprising a nanoemulsion and immunogen/antigen in only the prime or the one or more boost administrations, and uses a different immunogenic composition comprising the same or different immunogen and not comprising a nanoemulsion for the other delivery/administration. The invention is not limited by the other type of composition or platform utilized to deliver immunogen/antigen. Alternative compositions and platforms for delivery of immunogens are well known in the art and include, but are not limited to, delivery of antigen in a liposome, non-liposomal vaccine formulation, delivery of DNA vaccine encoding the antigen, delivery of a recombinant viral vaccine, a carrier molecule (e.g., proteins, polysaccharides, polylactic acids, polyglycollic acids, polymeric amino acids, amino acid copolymers, and inactive virus particles). Examples of particulate carriers include those derived from polymethyl methacrylate polymers, as well as microparticles derived from poly(lactides) and poly(lactide-co-glycolides), known as PLG. See, e.g., Jeffery et al., Pharm. Res. 10:362, 1993; McGee et al., J. Microencapsul. 14: 197, 1997; O'Hagan et al., Vaccine 11:149, 1993. Such carriers are well known to those of ordinary skill in the art.

In another embodiment, the invention provides a method of inducing an immune response in a subject (e.g., an immunogen-specific immune response, e.g., an immunogen-specific multi-component immune response) comprising providing a subject; and an immunogenic composition comprising a nanoemulsion and immunogen; and administering multiple deliveries via different routes of administration (e.g., administering via a first route (e.g., injection, e.g., parenterally, e.g., intramuscularly) and administering via a second route (e.g., mucosal administration, e.g., intranasally) the immunogenic composition to the subject to generate a desired immune response in the subject (e.g., an immunogen-specific immune response, e.g., an immunogen-specific multicomponent immune response, e.g., comprising a component induced by the first route and a component induced by the second route)). In such immunization protocols, a first route of delivery is a different route of administration than one or more second routes of deliveries of administration. As used herein, any particular reference to a “first route” and a “second route” indicates that the two routes are different. As such, as used herein a “first route” may be any route provided it is different than a “second route”; and, use of a “first route” or a “second route” to refer to a specific route (e.g., parenteral, mucosal, IN, IM, etc.) in one context does not preclude reference to a different specific route as a “first route” or a “second route” in another context. Accordingly, a specific route (e.g., parenteral, mucosal, IN, IM, etc.) may be referred to herein in some contexts as a “first route” and in other contexts as a “second route” and such references shall not be construed to be contradictory.

In preferred embodiments, one or more of the first route of administration and/or the second route of administration comprise(s) delivering an immunogenic composition of the invention to the subject via a mucosal route (e.g., intranasal, vaginal). In other preferred embodiments, one or more of the first route of administration and/or the second route of administration comprise(s) delivering to the subject an immunogenic composition of the invention to the subject via a parenteral route (e.g., infusion, injection, or implantation). The invention is not limited by the injectable route of administration. Indeed, any type of injection may be utilized including, but not limited to, subcutaneous, intramuscular, intraperitoneal, intradermal, and/or intravenous administration. In some preferred embodiments, intramuscular injection is utilized. In some embodiments, a first route of administration is via a mucosal route (e.g., nasal mucosa, genital mucosa, oral mucosa, rectal mucosa) and a second route of administration is via a parenteral route (e.g., intramuscular route). For example, in some preferred embodiments, a first route of administration is via an intranasal route and a second route of administration is via an intramuscular route (e.g., in order to generate an immunogen-specific, T helper type 17 (Th17) immune response).

In some preferred embodiments, the immune response generated via a first route of administration (e.g., a first component of a multi-component immune response) is qualitatively and/or quantitatively different than the immune response generated via a second route of administration (e.g., a second component of a multi-component immune response). For example, in one embodiment, a first route of administration via a mucosal route (e.g., nasal mucosa, genital mucosa, oral mucosa, rectal mucosa) generates an immune response in a subject characterized by a cytokine profile (e.g., elevated levels of Th17) and/or a T cell mediated immune response that is not obtained or observed utilizing administration via a second, parenteral route (intramuscular route). In another embodiment, a second route of administration via a parenteral route (e.g., intramuscular route) generates an immune response in a subject characterized by an immunogen-specific antibody titer (e.g., immunogen-specific IgG titer) that is not obtained or observed utilizing administration via a second, mucosal route (intranasal route). Thus, in a preferred embodiment, administration of an immunogenic composition of the invention via two or more routes of administration induces an immunogen-specific immune response (e.g., a multicomponent immune response) in a subject that is not attainable via administration of the immunogenic composition via only a single route. In some embodiments, the immunogen-specific immune response obtained provides superior neutralizing antibody capacity and/or ability to clear subsequent exposure to pathogens.

Accordingly, embodiments of the technology provide a method for inducing a multi-component immunogen-specific immune response in a subject, the method comprising: administering to the subject an immunogenic composition comprising a nanoemulsion and an immunogen via a first route to induce a first component of an immunogen-specific immune response and administering to the subject an immunogenic composition comprising a nanoemulsion and an immunogen via a second route to induce a second component of an immunogen-spocific immune response. For example, in some embodiments, the immunogenic composition is administered via a mucosal route of administration, e.g., in some embodiments the mucosal route of administration is via the nasal mucosa; in some embodiments the immunogenic composition is administered via a parenteral route of administration, e.g., in some embodiments the parenteral route of administration is selected from the group consisting of infusion, injection, and implantation. The technology is not limited in the type of injection, e.g., in some embodiments the injection is a subcutaneous injection, intramuscular injection, intradermal injection, intraperitoneal injection, and/or intravenous injection.

The technology provides for a multi-component immunogen-specific immune response. In some embodiments the first component of the immunogen-specific immune response is not attainable by administering to the subject an immunogenic composition comprising a nanoemulsion and an immunogen via the second route alone. In some embodiments the second component of the immunogen-specific immune response is not attainable by administering to the subject an immunogenic composition comprising a nanoemulsion and an immunogen via the first route alone. And, in some embodiments the multi-component immunogen-specific immune response is not attainable by administering to the subject an immunogenic composition comprising a nanoemulsion and an immunogen via the first route alone and/or in some embodiments the multi-component immunogen-specific immune response is not attainable by administering to the subject an immunogenic composition comprising a nanoemulsion and an immunogen via the second route alone.

The technology is not limited in the first and second routes used. For example, in some embodiments the first route is a mucosal route and the second route is an intramuscular route. Also, in some embodiments the same immunogenic composition is used for administering via the first route and for administering via the second route. For example, in some embodiments the immunogenic composition administered via the first route and the immunogenic composition administered via the second route comprise the same immunogen and the same nanoemulsion and the same amount of immunogen, but the percent of nanoemulsion present in the immunogenic composition administered via the first route is different than the percent of nanoemulsion present in the immunogenic composition administered via the second route. Additionally, some embodiments provide that the amount of immunogen present in the immunogenic composition administered via the first route is the same as the amount of immunogen present in the immunogenic composition administered via the second route.

The first and second components of the multi-component immune response comprise combinations of immune system entities such as antibodies, T cells, cytokines, and other immune system responses known in the art. For example, in some embodiments, the first component of the immunogen-specific immune response comprises induction of antibodies, cytokines, and/or a T cell response and the second component of the immunogen-specific immune response comprises a different induction of antibodies, cytokines, and/or a T cell response. In particular embodiments, the first component of the immunogen-specific immune response comprises a Th17 type immune response and in some embodiments the second component of the immunogen-specific immune response comprises an increased titer of IgG antibodies. For example, in some embodiments the second component of the immunogen-specific immune response comprises an increased titer of IgG antibodies that is 10 times to 100 times the titer of IgG antibodies of the first component of the immunogen-specific immune response.

The technology encompasses administrations (e.g., first administrations) via a first and second route and subsequent boost administrations (e.g., one or more second administrations) via a first and/or a second route. Accordingly, in some embodiments the methods further comprise one or both of administering to the subject a boost immunogenic composition comprising a nanoemulsion and an immunogen via the first route and/or administering to the subject a boost immunogenic composition comprising a nanoemulsion and an immunogen via the second route.

Further embodiments of the technology provide an immunization regimen for inducing a multi-component immunogen-specific immune response in a subject comprising (a) an immunogenic composition comprising a nanoemulsion and an immunogen for administration via a first route to induce a first component of an immunogen-specific immune response and (b) an immunogenic composition comprising a nanoemulsion and an immunogen for administration via a second route to induce a second component of an immunogen-spocific immune response and comprising the same nanoemulsion as in (a). In some embodiments of the immunization regimen, the same immunogen is present in both the immunogenic composition for administration via the first route and the immunogenic composition for administration via the second route. In some embodiments of the immunization regimen, the same immunogen is present in the same quantity in both the immunogenic composition for administration via the first route and the immunogenic composition for administration via the second route. The immunization regimen is not limited in the routes of administration for which it finds use. For example, in some embodiments of the immunization regimen the first route is a mucosal route, e.g., in some embodiments of the immunization regimen the mucosal route is via nasal mucosa. In some embodiments of the immunization regimen the second route is a parenteral route. In some embodiments, the first route is a mucosal route and the second route is an intramuscular injection. Moreover, in some embodiments the immunogenic composition for administration via the first route is the same as the immunogenic composition for administration via the second route.

In particular embodiments of the immunization regimen, the immunogenic composition administered via the first route and the immunogenic composition administered via the second route comprise the same immunogen and the same nanoemulsion and the same amount of immunogen, but the percent of nanoemulsion present in the immunogenic composition administered via the first route is different than the percent of nanoemulsion present in the immunogenic composition administered via the second route. In some embodiments of the immunization regimen, the immunogen present in the immunogenic composition administered via the first route is different than the immunogen present in the immunogenic composition administered via the second route. In some embodiments of the immunization regimen, the immunogenic composition for administration via the first route and the immunogenic composition for administration via the second route further comprise an adjuvant. In particular embodiments of the immunization regimen, the immunogen is a cancer antigen or a viral immunogen. For example, in some embodiments of the immunization regimen, the viral antigen is a respiratory syncytial virus (RSV) antigen, a herpes simplex virus (HSV) antigen, or an influenza antigen. In some embodiments of the immunization regimen, the immunogen is a bacterial antigen. In some embodiments of the immunization regimen the immunogen is a recombinant antigenic peptide, for example, in some embodiments the immunogen is a glycoprotein D2 subunit of HSV.

The invention is not limited by the duration of time between administrations of an immunogenic composition to a subject via a first route of administration and the administration of the same or different immunogenic composition via a second route of administration. In some embodiments, an immunogenic composition is administered via a first route and a second route at the same time. In some embodiments, an immunogenic composition is administered via a first route and within minutes is administered via a second route. In some embodiments, an immunogenic composition is administered via a first route and within hours is administered via a second route. In some embodiments, an immunogenic composition is administered via a first route and within days is administered via a second route. In some embodiments, an immunogenic composition is administered via a first route and within weeks is administered via a second route. In some embodiments, an immunogenic composition is administered via a first route and within months is administered via a second route.

In some embodiments, the same immunogenic composition is used for administrations to a subject via a first route of administration and for administration via a second route of administration. In some embodiments, a first immunogenic composition is used for administrations to a subject via a first route of administration and a second immunogenic composition is used for administration via a second route of administration. In some embodiments, the same nanoemulsion is used for administrations to a subject via a first route of administration and for administration via a second route of administration, but at a different dilution (e.g., an immunogenic composition comprising the same amount of immunogen and same nanoemulsion is used for both first and second routes of administration, but the percent of nanoemulsion present in the first route is different from the percent of nanoemulsion present in the second route). In some embodiments, a different nanoemulsion is used for the first route of administration than is used in a second route. In some embodiments, an immunogenic composition comprising the same amount of immunogen and same nanoemulsion is used for both first and second routes of administration. In some embodiments, the amount of immunogen administered to a subject via the immunogenic composition is the same for both first and second routes of administration. In some embodiments, the amount of immunogen administered to a subject via the immunogenic composition is different between the first and second routes of administration. In a preferred embodiment, the amount of immunogen/antigen delivered in a first and/or second route of administration is an effective amount to induce a desired immune response in a subject. The invention is not limited by the amount of immunogen/antigen delivered in a first and/or second route of administration. Indeed, any amount of immunogen/antigen may be delivered (e.g., independently or together with one or more different immunogens/antigens and/or adjuvants) to a subject including, but not limited to, those amounts disclosed herein. In some embodiments, a first amount of immunogen is utilized in a first route of administration, and a different, second amount of immunogen is utilized in a second route of administration (e.g., in order to generate a desired type and/or strength of immune response).

The invention is not limited by the type of immunogens/antigens delievered via the methods of the invention. Indeed, a variety of immunogens/antigens may be administered including, but not limited to, those disclosed herein. In accordance with an aspect of the present invention, there is provided an immunogenic composition for eliciting an immune response (e.g., a desired type (e.g., Th1, Th2, Th17, etc.) or strength (e.g., certain immunogen-specific antibody titer)) in a subject, the immunogenic composition comprising a nanoemulsion adjuvant described herein. The invention is not limited by the type of nanoemulsion utilized in an immunogenic composition administered. Indeed, any nanoemulsion may be utilized including, but not limited to, those disclosed herein.

In some embodiments of the present invention, there is provided a kit for preparing an immunogenic nanoemulsion adjuvant composition, comprising: (a) means for containing a nanoemulsion adjuvant; and (b) means for containing at least one antigen/immunogen; and (c) means for combining the nanoemulsion adjuvant and at least one antigen/immunogen to produce the immunogenic composition. The present invention provides several advantages over conventional adjuvants including, but not limited to, ease of formulation; effectiveness of adjuvanticity; lack of unwanted toxicity and/or host morbidity; and compatibility of antigens/immunogens with the adjuvant composition.

The present invention is not limited by the type of antigenic component (e.g., pathogen, pathogen component, antigen, immunogen, etc.) that can be utilized with (e.g., combined with, co-administered, administered before or after, etc.) a nanoemulsion adjuvant. In certain embodiments, the antigen/immunogen is selected from the group consisting of virus, bacteria, fungus and pathogen products derived from the virus, bacteria, or fungus. The present invention is not limited to a particular virus. A variety of viral immunogens are contemplated including, but not limited to, influenza A virus, avian influenza virus, H5N1 influenza virus, H1N1 influenza virus, West Nile virus, SARS virus, Marburg virus, Arenaviruses, Nipah virus, alphaviruses, filoviruses, herpes simplex virus I, herpes simplex virus II, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus. The present invention is not limited to a particular bacterium. A variety of bacterial immunogens are contemplated including, but not limited to, Bacillus cereus, Bacillus circulans and Bacillus megaterium, Bacillus anthracis, bacteria of the genus Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis. The present invention is also not limited to a particular fungus. A variety of fungal immunogens are contemplated including, but not limited to, Candida and Aspergillus.

In some embodiments, a nanoemulsion adjuvant provided herein skews an immune response toward a Th1 type response (e.g., when delivered via a prime/boost protocol described herein). In some embodiments, a nanoemulsion provided herein skews an immune response toward a Th2 type response (e.g., when delivered via a prime/boost protocol described herein). In some embodiments, a nanoemulsion provided herein skews an immune response toward a Th17 type response (e.g., when delivered via a prime/boost protocol described herein). In some embodiments, a nanoemulsion adjuvant provided herein provides a balanced Th1/Th2 response and/or polarization (e.g., an IgG subclass distribution and cytokine response indicative of a balanced Th1/Th2 response). Thus, a variety of immune responses may be generated and/or measured in a subject administered a nanoemulsion adjuvant of the present invention including, but not limited to, activation, proliferation and/or differentiation of cells of the immune system (e.g., B cells, T cells, dendritic cells, antigen presenting cells (APCs), macrophages, natural killer (NK) cells, etc.); up-regulated or down-regulated expression of markers and/or cytokines; stimulation of IgA, IgM, and/or IgG titers; splenomegaly (e.g., increased spleen cellularity); hyperplasia, mixed cellular infiltrates in various organs, and/or other responses (e.g., of cells) of the immune system that can be assessed with respect to immune stimulation known in the art.

In some embodiments, inducing an immune response primes the immune system of a host to respond to (e.g., to produce a Th1 and/or Th2 type response (e.g., thereby providing protective immunity to)) one or more pathogens (e.g., RSV, B. anthracis, vaccinia virus, C. botulinum, Y. pestis and/or HIV, etc.) in the host subject (e.g., human or animal subject). In some embodiments, the immunity comprises systemic immunity. In some embodiments, the immunity comprises mucosal immunity. In some embodiments, the immune response comprises increased expression of IFN-γ and/or TNF-α in the subject. In some embodiments, the immune response comprises a systemic IgG response. In some embodiments, the immune response comprises a mucosal IgA response. In some embodiments, the present invention provides an immunogenic composition for eliciting an immune response in a host, including a human, the composition comprising: (a) at least one antigen and/or immunogen; and (b) a nanoemulsion adjuvant. In some embodiments, the composition comprises an additional adjuvant (e.g., a second nanoemulsion adjuvant and/or a non-nanoemulsion adjuvant (e.g., CpG oligonucleotide, toxin, or other adjuvant described herein). The invention is not limited by the type of adjuvant utilized. Indeed a variety of adjuvants find use in the invention including, but not limited to, (1) aluminum salts (alum), such as aluminum hydroxide, aluminum phosphate, aluminum sulfate, etc.; (2) additional oil-in-water nanoemulsions disclosed herein; (3) one or more bacterial cell wall components such as monophosphorylipid A (MPL), trehalose dimycolate (TDM), and cell wall skeleton (CWS), preferably MPL+CWS (Detoxu); (4) saponin adjuvants, such as STIMULON (Cambridge Bioscience, Worcester, Mass.); (5) Complete Freunds Adjuvant (CFA) and Incomplete Freunds Adjuvant (IFA); (6) cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); (7) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); and (8) other substances that act as immunostimulating agents to enhance a subject's immune response. Additional adjuvants include pathogen-associated molecular patterns (PAMPs), which mediate innate immune activation via Toll-like Receptors (TLRs), (NOD)-like receptors (NLRs), Retinoic acid inducible gene-based (RIG)-1-like receptors (RLRs), and/or C-type lectin receptors (CLRs). Examples of PAMPs include lipoproteins, lipopolypeptides, peptidoglycans, zymosan, lipopolysaccharide, neisserial porins, flagellin, profillin, alpha-galactosylceramide, muramyl dipeptide. Peptidoglycans, lipoproteins, and lipoteichoic acids are cell wall components of Gram-positive. Lipopolysaccharides are expressed by most bacteria, with MPL being one example. Flagellin refers to the structural component of bacterial flagella that is secreted by pathogenic and commensal bacterial. alpha-Galactosylceramide (alpha-GalCer) is an activator of natural killer T (NKT) cells. Muramyl dipeptide is a bioactive peptidoglycan motif common to all bacteria. Other adjuvants include viral double-stranded RNA, which is sensed by the intracellular receptor TLR3; CpG motifs present on bacterial or viral DNA or ssRNA, which are sensed by TLR7, 8, and 9; all-trans retinoic acid; and heat shock proteins such as HSP70 and Gp96, which are highly effective carrier molecules for cross-presentation. Pharmaceutical adjuvants include resiquimod, a TLR7/8 agonists, and imiquimod, a TLR7 agonist.

In yet another aspect of the invention, there is provided a method of modulating and/or inducing an immune response (e.g., toward and/or away from a Th1 and/or Th2 type response) in a subject (e.g., toward an antigen) comprising providing a host subject and a nanoemulsion adjuvant composition of the invention, and administering the nanoemulsion adjuvant to the host subject under conditions such that an immune response is induced and/or modulated in the host subject. In some embodiments, the host immune response comprises enhanced expression and/or activity of Th1 type cytokines (e.g., IL-2, IL-12, IFN-γ and/or TNF-α, etc.) while concurrently lacking enhanced expression and/or activity of Th2 type cytokines (e.g., IL-4, IL-5, IL-10, etc.). In some embodiments, the host immune response comprises enhanced expression of Th2 type cytokines (e.g., IL-4, IL-5, IL-10, etc.) while concurrently lacking enhanced expression and/or activity of Th1 type cytokines (e.g., (e.g., IL-2, IL-12, IFN-γ and/or TNF-α, etc.). In some embodiments, the host immune response comprises enhanced expression and/or activity of Th17 type cytokines. In some embodiments, a nanoemulsion adjuvant composition administered to a subject induces expression and/or activity of Th1-type cytokines that increases to a greater extent than the level of expression and/or activity of Th2-type cytokines. For example, in some embodiments, a subject administered a nanoemulsion adjuvant composition induces a greater than 3 fold, greater than 5 fold, greater than 10 fold, greater than 20 fold, greater than 25 fold, greater than 30 fold or more enhanced expression of Th1 type cytokines (e.g., IL-2, IL-12, IFN-γ and/or TNF-α), with lower increases (e.g., less than 3 fold, less than two fold or less) enhanced expression of Th2 type cytokines (e.g., IL-4, IL-5, and/or IL-10). In some embodiments, a nanoemulsion adjuvant composition administered to a subject induces expression and/or activity of Th2-type cytokines that increases to a greater extent than the level of expression and/or activity of Th1-type cytokines. For example, in some embodiments, a subject administered a nanoemulsion adjuvant composition induces a greater than 3 fold, greater than 5 fold, greater than 10 fold, greater than 20 fold, greater than 25 fold, greater than 30 fold or more enhanced expression of Th2 type cytokines (e.g., IL-4, IL-5, and/or IL-10), with lower increases (e.g., less than 3 fold, less than two fold or less) enhanced expression of Th1 type cytokines (e.g., IL-2, IL-12, IFN-γ and/or TNF-α). In some embodiments, the host immune response comprises enhanced IL6 cytokine expression and/or activity while concurrently lacking enhanced expression and/or activity of other cytokines (e.g., IL4, TNF-α and/or IFN-γ) in the host. In some embodiments, the host immune response is specific for an antigen co-administered with the nanoemulsion adjuvant. In some embodiments, administering the nanoemulsion adjuvant to the host subject (e.g., in combination with an antigenic component (e.g., whole cell pathogen or component thereof)) induces and/or enhances the generation of one or more antibodies in the subject (e.g., IgG and/or IgA antibodies) that are not generated or generated at low levels in the host subject in the absence of administration of the nanoemulsion adjuvant. In some embodiments, administering the nanoemulsion adjuvant to the host induces a specific response to the nanoemulsion adjuvant by epithelial cells of the host. In some embodiments, administering the nanoemulsion adjuvant to the host induces uric acid and/or inflamasome activation in the host (e.g., that is distinguishable from uric acid and/or inflamasome activation induced by other types of adjuvants (e.g., alum adjuvants).

Antigens and/or immunogens that may be included in an immunogenic nanoemulsion adjuvant composition of the present invention, include, but are not limited to, microbial pathogens, bacteria, viruses, proteins, glycoproteins lipoproteins, peptides, glycopeptides, lipopeptides, toxoids, carbohydrates, and tumor-specific antigens. In some embodiments, mixtures of two or more antigens/immunogens may be utilized. Examples of immunogens and/or antigenic components of pathogens are described in detail herein.

In some embodiments, an immunogenic composition comprising a nanoemulsion is formulated to comprise between 0.1 and 500 μg of a protein antigen (e.g., derived or isolated from a pathogen and/or a recombinant form of an immunogenic pathogen component). However, the present invention is not limited to this amount of protein antigen. For example, in some embodiments, more than 500 μg of protein antigen is present in an immunogenic composition comprising nanoemulsion for administration to a subject. In some embodiments, less than 0.1 μg of protein antigen is present in an immunogenic composition comprising nanoemulsion for administration to a subject. In some embodiments, a pathogen (e.g., a virus) is inactivated by the nanoemulsion adjuvant and is then administered to the subject under conditions such that between about 10 and 10⁷ pfu (e.g., about 10², 10³, 10⁴, 10⁵, or 10⁶ pfu) of the inactivated pathogen is present in a dose administered to the subject. However, the present invention is not limited to this amount of pathogen present in an immunogenic composition comprising nanoemulsion administered. For example, in some embodiments, more than 10⁷ pfu of the inactivated pathogen (e.g., 10⁸ pfu, 10⁹ pfu, or more) is present in a dose administered to the subject.

In some embodiments, the present invention provides an immunogenic composition comprising nanoemulsion comprising a 10% nanoemulsion. However, the present invention is not limited to this amount (e.g., percentage) of nanoemulsion. For example, in some embodiments, an immunogenic composition comprising nanoemulsion comprises less than 10% nanoemulsion (e.g., 9%, 8%, 7%, 6%, 5%, 4%, 3%, 2%, 1%, 0.5% or less). In some embodiments, a composition comprises more than 10% nanoemulsion (e.g., 15%, 20%, 25%, 30%, 35%, 40%. 45%, 50%, 60%, 70% or more). In some embodiments, an immunogenic composition comprising nanoemulsion of the present invention comprises any of the nanoemulsions described herein. In some embodiments, the nanoemulsion comprises W₂₀5EC. In some embodiments, the nanoemulsion comprises W₈₀5EC. In some embodiments, the nanoemulsion is X8P. In some embodiments, the nanoemulsion comprises P₄₀₇5EC. In some embodiments, immune responses resulting from administration of an immunogenic composition comprising nanoemulsion (e.g., individually and/or in combination with immunogenic pathogen components) protects the subject from displaying signs or symptoms of disease caused by a pathogen (e.g., vaccinia virus, B. anthracis, HIV, etc.). In some embodiments, immune responses resulting from administration of a nanoemulsion adjuvant (e.g., individually and/or in combination with immunogenic pathogen components) reduces the risk of infection upon one or more exposures to a pathogen. In some embodiments, administration of a nanoemulsion adjuvant to a host subject (e.g., in combination with an antigenic component (e.g., whole cell pathogen or component thereof)) induces the generation of one or more antibodies in the subject (e.g., IgG and/or IgA antibodies) that are not generated in the host subject in the absence of administration of the nanoemulsion adjuvant.

### BRIEF DESCRIPTION OF THE DRAWINGS

The following figures form part of the present specification and are included to further demonstrate certain aspects and embodiments of the present invention. The invention may be better understood by reference to one or more of these figures in combination with the description of specific embodiments presented herein.

FIG. 1 shows that route of NE administration drives type of immune response when an immunogenic composition comprising nanoemulsion and respiratory syncytial virus (NE-RSV) is administered.

FIG. 2 shows that heterologous prime/boost strategy enhances production of Th1-type cytokines in response to HBsAg.

FIG. 3 shows a strong Th17 response via intranasal but not intramuscular route, and that IN/IM heterologous prime/boost strategy maintains Th17 type immune response.

FIG. 4 shows that heterologous prime/boost strategy enhances production of Th2-type cytokines.

FIG. 5 shows that heterologous prime/boost strategy enhances anti-HBsAg serum IgG response compared to IN route alone.

FIG. 6 shows that heterologous prime/boost strategy enhances anti-HBsAg-specific IgG antibody responses in Bronchial Alveolar Lavage (BAL) compared to IN route alone.

FIG. 7 is a plot showing that one or three immunizations IM induced a higher serum antibody titer than three IN immunizations.

FIG. 8 is a plot showing that one or three immunizations IM induced a higher serum neutralizing activity than three IN immunizations.

FIG. 9 is a plot showing that the specific neutralizing activity of serum after IN immunization and the specific neutralizing activity of serum after IM immunization are the same.

FIG. 10 is a plot showing that both IN and IM immunized animals completely cleared a challenge by live virus infection.

FIG. 11 is a plot showing that an IN immunization does not prime a subsequent IM immunization and that an IM immunization does not prime a subsequent IN immunization.

FIG. 12 is a plot showing that IM immunization produces a higher neutralization activity in serum than IN immunization.

FIG. 13 is a plot showing that both IM and IN immunization induced a similar protection and clearing of a vaginal infection challenge.

FIG. 14 is a plot showing that both IM and IN immunization induced a similar protection against recurrence of infection post-acute phase.

### GENERAL DESCRIPTION OF THE INVENTION

The present invention provides methods and compositions for the stimulation of immune responses. In particular, the present invention provides immunogenic nanoemulsion compositions and methods of administering the same (e.g., via a heterologous prime/boost protocol (e.g., utilizing the same nanoemulsion in each the prime and boost administrations)) to induce immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

In one embodiment, the invention provides a method of inducing an immune response in a subject (e.g., an immunogen-specific immune response) comprising providing a subject; and an immunogenic composition comprising a nanoemulsion and immunogen; and administering multiple deliveries (e.g., via a prime/boost protocol) of the immunogenic composition to the subject in order to generate a desired immune response in the subject (e.g., an immunogen-specific immune response). In such immunization protocols, a priming delivery may be via a different route of administration than one or more boost deliveries. In preferred embodiments, one or more of the prime and boost deliveries comprises delivering to the subject via a mucosal route (e.g., nasal mucosa, genital mucosa, oral mucosa, rectal mucosa) an immunogenic composition of the invention. In other preferred embodiments, one or more of the prime and boost deliveries comprises delivering to the subject via a parenteral route (e.g., infusion, injection or implantation) an immunogenic composition of the invention. The invention is not limited by the injectable route of administration. Indeed, any type of injection may be utilized including, but not limited to, subcutaneous, intramuscular, intraperitoneal, and/or intravenous administration. In some preferred embodiments, intramuscular injection is utilized. In some embodiments, a prime administration is via a mucosal route (e.g., intranasal, vaginal) and a boost administration is via an intramuscular route. For example, in some preferred embodiments, a prime administration is via an intranasal route and a boost administration is via an intramuscular route (e.g., in order to generate an immunogen-specific, T helper type 17 (Th17) immune response

The present invention provides immunogenic compositions comprising nanoemulsion and methods of using the same (e.g., individually, or together with one or more antigens/immunogens (e.g., pathogens (e.g., RSV, vaccinia virus, H5N1 influenza virus, Bacillus anthracis, C. botulinum, Y. pestis, Hepatitis B, and/or HIV, etc.) or components thereof (e.g., recombinant proteins therefrom), in a prime/boost scheme or protocol, to induce an immune response in a subject (e.g., to prime, enable and/or enhance an immune response (e.g., against one or a plurality of pathogens in a subject)). In some embodiments, an immunogenic composition comprising nanoemulsion of the present invention is utilized by itself, or together with another adjuvant (e.g., another nanoemulsion adjuvant and/or non-nanoemulsion adjuvant) in the absence of an antigen/immunogen present in the emulsion to stimulate an immune response (e.g., innate immune response and/or adaptive immune response) in a host subject. In some embodiments, one or a plurality of pathogens is mixed with a nanoemulsion prior to administration for a time period sufficient to inactivate the one or plurality of pathogens. In some embodiments, one or a plurality of protein components (e.g., isolated and/or purified and/or recombinant protein) from one or a plurality of pathogens is mixed with the nanoemulsion.

Although an understanding of the mechanism is not necessary to practice the present invention and the present invention is not limited to any particular mechanism of action, in some embodiments, an immunogenic composition comprising nanoemulsion penetrates mucosa to which it is administered (e.g., through pores) and carry immunogens to submucosal locations (e.g., harboring dendritic cells (e.g., thereby initiating and/or stimulating an immune response)). In some embodiments, an immunogenic composition comprising nanoemulsion of the invention preserves and/or stabilizes antigenic epitopes (e.g., recognizable by a subject's immune system), stabilizing their hydrophobic and/or hydrophilic components in the emulsion (e.g., thereby providing one or more immunogens (e.g., stabilized antigens) against which a subject can mount an immune response). In some embodiments, an immunogenic composition comprising nanoemulsion of the invention (e.g., comprising one or more protein and/or cellular antigens) creates an environment in which a protein or cellular antigen is maintained for a longer period of time in a subject (e.g., thereby providing enhanced opportunity for the protein or cellular antigen to be recognized and responded to by a host immune system). Although an understanding of the mechanism is not necessary to practice the present invention and the present invention is not limited to any particular mechanism of action, in some embodiments, dendritic cells avidly phagocytose nanoemulsion (NE) oil droplets and provide a means to prime, enable and/or enhance host immune responses (e.g., toward a Th1, Th2 and/or Th17 type response, and/or to internalize immunogens (e.g., antigenic proteins or peptide fragments thereof present in the adjuvant) for antigen presentation). While other vaccines rely on inflammatory toxins or other immune stimuli for adjuvant activity (See, e.g., Holmgren and Czerkinsky, Nature Med. 2005, 11; 45-53), nanoemulsions (NEs) have not been shown to be inflammatory when placed on the skin or mucous membranes in studies on animals and in humans. Thus, although an understanding of the mechanism is not necessary to practice the present invention and the present invention is not limited to any particular mechanism of action, in some embodiments, an immunogenic composition comprising nanoemulsion of the present invention (e.g., a composition comprising NE adjuvant optionally combined with one or more immunogens (e.g., a NE adjuvant inactivated pathogen (e.g., a virus (e.g., VV))) acts as a “physical” adjuvant (e.g., that transports and/or presents antigens/immunogens or the nanoemulsion adjuvant itself to the immune system. In some embodiments, mucosal administration of a composition of the present invention generates mucosal (e.g., signs of mucosal immunity (e.g., generation of IgA antibody titers)) as well as systemic immunity. In some embodiments, mucosal administration of a nanoemulsion adjuvant composition of the invention generates an innate immune response (e.g., activates Toll-like receptor signaling and/or activation of NF-kB) in a subject.

Both cellular and humoral immunity play a role in protection against multiple pathogens and both can be induced with the immunogenic compositions comprising nanoemulsion of the present invention. For example, vaccinia-specific antibody titers are considered important for the estimate of protective immunity in human subjects and in animal models of vaccination (See, e.g., Hammarlund et al, Nat. Med. 2003, 9; 1131-1137). Several studies have identified proteins important for the elicitation of neutralizing antibodies (See, e.g., Galmiche et al, Virology, 1999, 254; 71-80; Hooper et al, Virology, 2003, 306; 181-195). A recent trial of dilutions of the licensed smallpox vaccine (Dryvax) in human volunteers, confirmed that pustule formation strongly correlated with development of both specific antibodies and induction of cytotoxic T lymphocytes (CTL) and elevated INF-γ T cell responses (See, e.g., Greenberg et al, 2005, 365; 398-409). Induction of IFN-γ is suggestive of activation of specific MHC class I-restricted CD8+ T cells. These types of cells have been implicated in the recognition and clearance of Vaccinia infected cells, and for maintenance of immunity after vaccination (See, e.g., Earl et al, Nature, 2004; 482; 182-185; Hammarlund et al, Nat. Med. 2003, 9; 1131-1137; Edghill-Smith et all, Nature Med. 2005, 11; 740-747).

Thus, in some embodiments, administration (e.g., mucosal administration) of an immunogenic composition comprising nanoemulsion of the present invention primes, enables and/or enhances induction of both humoral (e.g., development of specific antibodies) and cellular (e.g., cytotoxic T lymphocyte) immune responses (e.g., against a pathogen). In some embodiments, an immunogenic composition comprising nanoemulsion of the present invention is used in a vaccine (e.g., as an immunostimulatory adjuvant (e.g., that elicits and/or enhances immune responses (e.g., innate and or adaptive immune responses) in a host administered the nanoemulsion adjuvant). Furthermore, in some embodiments, a composition of the present invention (e.g., an immunogenic composition comprising nanoemulsion) induces (e.g., when administered to a subject) both systemic and mucosal immune responses (e.g., generates systemic and or mucosal immunity). Thus, in some embodiments, administration of a composition of the present invention to a subject results in protection against an exposure (e.g., a lethal mucosal exposure) to one or a plurality of pathogens (e.g., one or a plurality of viruses and/or bacteria). Although an understanding of the mechanism is not necessary to practice the present invention and the present invention is not limited to any particular mechanism of action, mucosal administration provides protection against pathogen infection (e.g., that initiates at a mucosal surface). Although it has heretofore proven difficult to stimulate secretory IgA responses and protection against pathogens that invade at mucosal surfaces (See, e.g., Mestecky et al, Mucosal Immunology. 3ed edn. (Academic Press, San Diego, 2005)), the present invention provides compositions and methods for stimulating mucosal immunity (e.g., a protective IgA response) against one or a plurality of pathogens in a subject.

In some embodiments, the present invention provides immunogenic compositions comprising nanoemulsion that replace the use of other adjuvants (e.g., adjuvants that cause inflammation, morbidity, and/or adverse side reactions in a host administered the composition). For example, in some embodiments, a nanoemulsion of the invention is utilized in an immunogenic composition (e.g., a vaccine) in place of a Th1-type adjuvant. In some embodiments, a nanoemulsion of the invention is utilized in an immunogenic composition (e.g., a vaccine) in place of a Th2-type adjuvant. In some embodiments, a nanoemulsion of the invention provides, when administered to a host subject (e.g., via a heterologous prime/boost protocol described herein), an immune response (e.g., an innate, cell mediated, adaptive and/or acquired immune response) that is similar to, the same as, or greater than an immune response elicited by a conventional adjuvant compositions (e.g., cholera toxin, CpG oligonucleotide, alum, and/or other adjuvant described herein) without adverse and/or unwanted side-effects.

### DEFINITIONS

To facilitate an understanding of the present invention, a number of terms and phrases are defined below:

As used herein, the term “microorganism” refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

As used herein the term “pathogen,” and grammatical equivalents, refers to an organism (e.g., biological agent), including microorganisms, that causes a disease state (e.g., infection, pathologic condition, disease, etc.) in another organism (e.g., animals and plants) by directly infecting the other organism, or by producing agents that causes disease in another organism (e.g., bacteria that produce pathogenic toxins and the like). “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc.

As used herein, the term “fungi” is used in reference to eukaryotic organisms such as molds and yeasts, including dimorphic fungi.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

The terms “host” or “subject,” as used herein, refer to an individual to be treated by (e.g., administered) the compositions and methods of the present invention. Subjects include, but are not limited to, mammals (e.g., murines, simians, equines, bovines, porcines, canines, felines, and the like), and most preferably includes humans. In the context of the invention, the term “subject” generally refers to an individual who will be administered or who has been administered one or more compositions of the present invention (e.g., a composition for inducing an immune response).

As used herein, the terms “inactivating,” “inactivation” and grammatical equivalents, when used in reference to a microorganism (e.g., a pathogen (e.g., a bacterium or a virus)), refer to the killing, elimination, neutralization and/or reducing of the capacity of the microorganism (e.g., a pathogen (e.g., a bacterium or a virus)) to infect and/or cause a pathological response and/or disease in a host. For example, in some embodiments, the present invention provides a composition comprising nanoemulsion (NE)-inactivated vaccinia virus (VV). Accordingly, as referred to herein, compositions comprising “NE-inactivated VV,” “NE-killed V,” NE-neutralized V″ or grammatical equivalents refer to compositions that, when administered to a subject, are characterized by the absence of, or significantly reduced presence of, VV replication (e.g., over a period of time (e.g., over a period of days, weeks, months, or longer)) within the host.

As used herein, the term “fusigenic” is intended to refer to an emulsion that is capable of fusing with the membrane of a microbial agent (e.g., a bacterium or bacterial spore). Specific examples of fusigenic emulsions are described herein.

As used herein, the term “lysogenic” refers to an emulsion (e.g., a nanoemulsion) that is capable of disrupting the membrane of a microbial agent (e.g., a virus (e.g., viral envelope) or a bacterium or bacterial spore). In preferred embodiments of the present invention, the presence of a lysogenic and a fusigenic agent in the same composition produces an enhanced inactivating effect compared to either agent alone. Methods and compositions (e.g., for inducing an immune response (e.g., used as a vaccine) using this improved antimicrobial composition are described in detail herein.

The term “emulsion,” as used herein, includes classic oil-in-water or water in oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in some embodiments, the nanoemulsions comprise an oil phase having droplets with a mean particle size of approximately 0.1 to 5 microns (e.g., about 150, 200, 250, 300, 350, 400, 450, 500 nm or larger in diameter), although smaller and larger particle sizes are contemplated. The terms “emulsion” and “nanoemulsion” are often used herein, interchangeably, to refer to the nanoemulsions of the present invention.

As used herein, the terms “contact,” “contacted,” “expose,” and “exposed,” when used in reference to a nanoemulsion and a live microorganism, refer to bringing one or more nanoemulsions into contact with a microorganism (e.g., a pathogen) such that the nanoemulsion inactivates the microorganism or pathogenic agent, if present. The present invention is not limited by the amount or type of nanoemulsion used for microorganism inactivation. A variety of nanoemulsion that find use in the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes). Ratios and amounts of nanoemulsion (e.g., sufficient for inactivating the microorganism (e.g., virus inactivation)) and microorganisms (e.g., sufficient to provide an antigenic composition (e.g., a composition capable of inducing an immune response)) are contemplated in the present invention including, but not limited to, those described herein.

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described, for example, by Meyers, (See, e.g., Meyers, Surfactant Science and Technology, VCH Publishers Inc., New York, pp. 231-245 (1992)), incorporated herein by reference. As used herein where appropriate, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water that are good solubilizers of water in oils are at the low end of the scale.

As used herein the term “interaction enhancers” refers to compounds that act to enhance the interaction of an emulsion with a microorganism (e.g., with a cell wall of a bacteria (e.g., a Gram negative bacteria) or with a viral envelope (e.g., Vaccinia virus envelope)). Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum albumin (BSA) and the like).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “a composition for inducing an immune response” refers to a composition that, once administered to a subject (e.g., once, twice, three times or more (e.g., separated by weeks, months or years)), stimulates, generates and/or elicits an immune response in the subject (e.g., resulting in total or partial immunity to a microorganism (e.g., pathogen) capable of causing disease). In preferred embodiments of the invention, the composition comprises a nanoemulsion and an immunogen. In further preferred embodiments, the composition comprising a nanoemulsion and an immunogen comprises one or more other compounds or agents including, but not limited to, therapeutic agents, physiologically tolerable liquids, gels, carriers, diluents, adjuvants, excipients, salicylates, steroids, immunosuppressants, immunostimulants, antibodies, cytokines, antibiotics, binders, fillers, preservatives, stabilizing agents, emulsifiers, and/or buffers. An immune response may be an innate (e.g., a non-specific) immune response or a learned (e.g., acquired) immune response (e.g. that decreases the infectivity, morbidity, or onset of mortality in a subject (e.g., caused by exposure to a pathogenic microorganism) or that prevents infectivity, morbidity, or onset of mortality in a subject (e.g., caused by exposure to a pathogenic microorganism)). Thus, in some preferred embodiments, a composition comprising a nanoemulsion and an immunogen is administered to a subject as a vaccine (e.g., to prevent or attenuate a disease (e.g., by providing to the subject total or partial immunity against the disease or the total or partial attenuation (e.g., suppression) of a sign, symptom or condition of the disease.

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, the nanoemulsion formulations described herein, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present invention (e.g., comprising HIV or an immunogenic epitope thereof (e.g., gp120)) are administered with one or more adjuvants (e.g., to skew the immune response towards a Th1 and/or Th2 type response).

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

A used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the terms “toll receptors” and “TLRs” refer to a class of receptors (e.g., TLR1, TLR2, TLR3, TLR4, TLR5, TLR6, TLR7, TLR8, TLR9, TLRT0, TLR 11) that recognize special patterns of pathogens, termed pathogen-associated molecular patterns (See, e.g., Janeway and Medzhitov, (2002) Annu. Rev. Immunol. 20, 197-216). These receptors are expressed in innate immune cells (e.g., neutrophils, monocytes, macrophages, dendritic cells) and in other types of cells such as endothelial cells. Their ligands include bacterial products such as LPS, peptidoglycans, lipopeptides, and CpG DNA. TLRs are receptors that bind to exogenous ligands and mediate innate immune responses leading to the elimination of invading microbes. The TLR-triggered signaling pathway leads to activation of transcription factors including NFkB, which is important for the induced expression of proinflammatory cytokines and chemokines TLRs also interact with each other. For example, TLR2 can form functional heterodimers with TLR1 or TLR6. The TLR2/1 dimer has different ligand binding profile than the TLR2/6 dimer (Ozinsky et al., 2000). In some embodiments, a nanoemulsion adjuvant activates cell signaling through a TLR (e.g., TLR2 and/or TLR4). Thus, methods described herein include a nanoemulsion adjuvant composition (e.g., composition comprising NE adjuvant optionally combined with one or more immunogens (e.g., proteins and/or NE adjuvant inactivated pathogen (e.g., a virus (e.g., VV)))) that when administered to a subject, activates one or more TLRs and stimulates an immune response (e.g., innate and/or adaptive/acquired immune response) in a subject. Such an adjuvant can activate TLRs (e.g., TLR2 and/or TLR4) by, for example, interacting with TLRs (e.g., NE adjuvant binding to TLRs) or activating any downstream cellular pathway that occurs upon binding of a ligand to a TLR. NE adjuvants described herein that activate TLRs can also enhance the availability or accessibility of any endogenous or naturally occurring ligand of TLRs. A NE adjuvant that activates one or more TLRs can alter transcription of genes, increase translation of mRNA or increase the activity of proteins that are involved in mediating TLR cellular processes. For example, NE adjuvants described herein that activate one or more TLRs (e.g., TLR2 and/or TLR4) can induce expression of one or more cytokines (e.g., IL-8, IL-12p40, and/or IL-23)

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the terms “immunogen” and “antigen” refer to an agent (e.g., a microorganism (e.g., bacterium, virus or fungus) and/or portion or component thereof (e.g., a protein antigen (e.g., gp120 or rPA))) that is capable of eliciting an immune response in a subject. In preferred embodiments, immunogens elicit immunity against the immunogen (e.g., microorganism (e.g., pathogen or a pathogen product)) when administered in combination with a nanoemulsion of the present invention.

As used herein, the term “pathogen product” refers to any component or product derived from a pathogen including, but not limited to, polypeptides, peptides, proteins, nucleic acids, membrane fractions, and polysaccharides.

As used herein, the term “enhanced immunity” refers to an increase in the level of adaptive and/or acquired immunity in a subject to a given immunogen (e.g., microorganism (e.g., pathogen)) following administration of a composition (e.g., composition for inducing an immune response of the present invention) relative to the level of adaptive and/or acquired immunity in a subject that has not been administered the composition (e.g., composition for inducing an immune response of the present invention).

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

As used herein, the terms “administration” and “administering” refer to the act of giving a composition of the present invention (e.g., a composition for inducing an immune response (e.g., a composition comprising a nanoemulsion and an immunogen)) to a subject. Exemplary routes of administration to the human body include, but are not limited to, through the eyes (ophthalmic), mouth (oral), skin (transdermal), nose (nasal), lungs (inhalant), oral mucosa (buccal), ear, rectal, by injection (e.g., intravenously, subcutaneously, intraperitoneally, etc.), topically, and the like.

As used herein, the terms “co-administration” and “co-administering” refer to the administration of at least two agent(s) (e.g., a composition comprising a nanoemulsion and an immunogen and one or more other agents—e.g., an adjuvant) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. In other embodiments, co-administration is preferable to elicit an immune response in a subject to two or more different immunogens (e.g., microorganisms (e.g., pathogens)) at or near the same time (e.g., when a subject is unlikely to be available for subsequent administration of a second, third, or more composition for inducing an immune response).

As used herein, the term “topically” refers to application of a compositions of the present invention (e.g., a composition comprising a nanoemulsion and an immunogen) to the surface of the skin and/or mucosal cells and tissues (e.g., alveolar, buccal, lingual, masticatory, vaginal or nasal mucosa, and other tissues and cells which line hollow organs or body cavities).

In some embodiments, the compositions of the present invention are administered in the form of topical emulsions, injectable compositions, ingestible solutions, and the like. When the route is topical, the form may be, for example, a spray (e.g., a nasal spray), a cream, or other viscous solution (e.g., a composition comprising a nanoemulsion and an immunogen in polyethylene glycol).

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk (e.g., a subject may be “at risk for disease” simply by being exposed to and interacting with other people), nor is it intended that the present invention be limited to any particular disease.

“Nasal application”, as used herein, means applied through the nose into the nasal or sinus passages or both. The application may, for example, be done by drops, sprays, mists, coatings or mixtures thereof applied to the nasal and sinus passages.

“Vaginal application”, as used herein, means applied into or through the vagina so as to contact vaginal mucosa. The application may contact the urethra, cervix, fornix, uterus or other area surrounding the vagina. The application may, for example, be done by drops, sprays, mists, coatings, lubricants or mixtures thereof applied to the vagina or surrounding tissue.

As used herein, the term “kit” refers to any delivery system for delivering materials. In the context of immunogenic agents (e.g., compositions comprising a nanoemulsion and an immunogen), such delivery systems include systems that allow for the storage, transport, or delivery of immunogenic agents and/or supporting materials (e.g., written instructions for using the materials, etc.) from one location to another. For example, kits include one or more enclosures (e.g., boxes) containing the relevant immunogenic agents (e.g., nanoemulsions) and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a nanoemulsion and an immunogen for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of an immunogenic agent needed for a particular use in a single container (e.g., in a single box housing each of the desired components). The term “kit” includes both fragmented and combined kits.

### DETAILED DESCRIPTION OF THE INVENTION

The present invention provides methods and compositions for the stimulation of immune responses. In particular, the present invention provides immunogenic nanoemulsion compositions and methods of administering the same (e.g., via a heterologous prime/boost protocol (e.g., utilizing the same nanoemulsion in each the prime and boost administrations)) to induce immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

In one embodiment, the invention provides a method of inducing an immune response in a subject (e.g., an immunogen-specific immune response) comprising providing a subject; and an immunogenic composition comprising a nanoemulsion and immunogen; and administering multiple deliveries (e.g., via a prime/boost protocol or administration via a first route of administration and administration via a second route of administration) of the immunogenic composition to the subject in order to generate a desired immune response in the subject (e.g., an immunogen-specific immune response). In such immunization protocols, a priming delivery may be via a different route of administration than one or more boost deliveries. In preferred embodiments, one or more of the prime and boost deliveries comprises delivering to the subject via a mucosal route (e.g., intranasal, vaginal) an immunogenic composition of the invention. In other preferred embodiments, one or more of the prime and boost deliveries comprises delivering to the subject via a parenteral route (e.g., infusion, injection or implantation) an immunogenic composition of the invention. The invention is not limited by the injectable route of administration. Indeed, any type of injection may be utilized including, but not limited to, subcutaneous, intramuscular, intraperitoneal, and/or intravenous administration. In some preferred embodiments, intramuscular injection is utilized. In some embodiments, a prime administration is via a mucosal route (e.g., nasal mucosa, genital mucosa, oral mucosa, rectal mucosa) and a boost administration is via an intramuscular route. For example, in some preferred embodiments, a prime administration is via an intranasal route and a boost administration is via an intramuscular route (e.g., in order to generate an immunogen-specific, T helper type 17 (Th17) immune response). In some embodiments, the same immunogenic composition is used for both the prime and subsequent boost administrations/deliveries. In a preferred embodiment, the same nanoemulsion is used for both the prime and subsequent boost administrations/deliveries. In some embodiments, the same nanoemulsion is used for both the prime and subsequent boost administrations/deliveries, but at a different dilution (e.g., an immunogenic composition comprising the same amount of immunogen and same nanoemulsion is used for both prime and boost administrations, but the percent of nanoemulsion present in the prime administration is different from the percent of nanoemulsion present in the boost administration). In some embodiments, a different nanoemulsion is used for the prime administration than is used in a subsequent boost administration/delivery. In some embodiments, an immunogenic composition comprising the same amount of immunogen and same nanoemulsion is used for both prime and boost administrations. In some embodiments, the amount of immunogen administered to a subject via the immunogenic composition is the same for both prime and boost administrations/deliveries. In some embodiments, the amount of immunogen administered to a subject via the immunogenic composition is different between the prime and boost administrations/deliveries. In a preferred embodiment, the amount of immunogen/antigen delivered in a prime and/or boost administration is an effective amount to induce a desired immune response in a subject. The invention is not limited by the amount of immunogen/antigen delivered in a prime and/or boost administration. Indeed, any amount of immunogen/antigen may be delivered (e.g., independently or together with one or more different immunogens/antigens and/or adjuvants) to a subject including, but not limited to, those amounts disclosed herein. In some embodiments, a first amount of immunogen is utilized in a prime administration/delivery, and a different, second amount of immunogen is utilized in a boost administration/delivery (e.g., in order to generate a desired type and/or strength of immune response). The invention is not limited by the type of immunogens/antigens delievered via a method of the invention. Indeed, a variety of immunogens/antigens may be administered including, but not limited to, those disclosed herein. In some embodiments, the antigen is a virus or component (e.g., a protein, peptide, nucleic acid, etc.) from a virus. In some embodiments, the antigen in a herpes simplex virus antigen (e.g., herpes simplex virus II). In a preferred embodiment, the antigen is a respiratory syncytial virus (RSV) antigen. In accordance with an aspect of the present invention, there is provided an immunogenic composition for eliciting an immune response (e.g., a desired type (e.g., Th1, Th2, Th17, etc.) or strength (e.g., certain immunogen-specific antibody titer)) in a subject, the immunogenic composition comprising a nanoemulsion adjuvant described herein. The invention is not limited by the type of nanoemulsion utilized in an immunogenic composition administered. Indeed, any nanoemulsion may be utilized including, but not limited to, those disclosed herein.

For example, in one aspect of the invention, there is provided a method of generating an immune response in a subject comprising administering thereto an immunogenic nanoemulsion composition of the present invention (e.g., independently and/or in combination with one or more antigenic (e.g., microbial pathogen (e.g., bacteria, viruses, etc.) protein, glycoprotein, lipoprotein, peptide, glycopeptide, lipopeptide, toxoid, carbohydrate, tumor-specific antigen))) components. In some embodiments, a host immune response attained via administration of a nanoemulsion adjuvant to a host subject is a humoral immune response. In some embodiments, a host immune response attained via administration of a nanoemulsion adjuvant to a host subject is a cell-mediated immune response. In some embodiments, a host immune response attained via administration of a nanoemulsion adjuvant to a host subject is an innate immune response. In some embodiments, a host immune response attained via administration of a nanoemulsion adjuvant to a host subject is a combination of innate, cell-mediated, and/or humoral immune responses. In some embodiments, a composition comprising a nanoemulsion adjuvant further comprises a pharmaceutically acceptable carrier.

In some embodiments, the prime and one or more boost deliveries of an immunogen/antigen utilizes an immunogenic composition comprising a nanoemulsion and immunogen/antigen. In other embodiments, the prime and one or more boost deliveries of an immunogen/antigen utilizes an immunogenic composition comprising a nanoemulsion and immunogen/antigen in only the prime or the one or more boost administrations, and uses a different immunogenic composition comprising the same or different immunogen and not comprising a nanoemulsion for the other delivery/administration. The invention is not limited by the other type of composition or platform utilized to deliver immunogen/antigen. Alternative compositions and platforms for delivery of immunogens are well known in the art and include, but are not limited to, delivery of antigen in a liposome, non-liposomal vaccine formulation, delivery of DNA vaccine encoding the antigen, delivery of a recombinant viral vaccine, a carrier molecules (e.g., proteins, polysaccharides, polylactic acids, polyglycollic acids, polymeric amino acids, amino acid copolymers, and inactive virus particles). Examples of particulate carriers include those derived from polymethyl methacrylate polymers, as well as microparticles derived from poly(lactides) and poly(lactide-co-glycolides), known as PLG. See, e.g., Jeffery et al., Pharm. Res. 10:362, 1993; McGee et al., J. Microencapsul. 14: 197, 1997; O'Hagan et al., Vaccine 11:149, 1993. Such carriers are well known to those of ordinary skill in the art.

A prime and a boost administration of an immunogenic composition comprising a nanoemulsion of the invention can be administered by any one or combination of the following routes. In one aspect, the prime and boost are administered by the same route. In another aspect, the prime and boost are administered by different routes (e.g., a first route and a second route that is different than the first route). The term “different routes” encompasses, but is not limited to, different sites on the body, for example, a site that is oral, non-oral, enteral, parenteral, rectal, intranode (lymph node), intravenous, arterial, subcutaneous, intramuscular, intratumor, peritumor, intratumor, infusion, mucosal, nasal, in the cerebrospinal space or cerebrospinal fluid, and so on, as well as by different modes, for example, oral, intravenous, and intramuscular.

During the development of embodiments of the technology provided herein, data were collected demonstrating that an immune response induced by administration of an immunogenic composition (e.g., an immunogenic composition comprising a nanoemulsion and an antigen) via a mucosal route (e.g., an intranasal or IN route) is different (e.g., comprises different components) than an immune response induced by administration of the same immunogenic composition (e.g., the immunogenic composition comprising the nanoemulsion and the antigen) via a parenteral (e.g., an intramuscular or IM) route. For example, in some embodiments, the immune response induced via mucosal administration of an immunogenic composition comprises production of lower (e.g., 10%, 20%, 30% 40%, 50%, 60%, 70%, 80%, 90%; 1/10, 1/9, ⅛, 1/7, ⅙, ⅕, ¼, ⅓, ½) antibody titers (e.g., lower serum IgG) than the immune response induced via parenteral administration of the same immunogenic composition. However, despite these differences in antibody titers, immunization via an IN route and immunization via an IM route provide the same or similar protection against infection (e.g., neutralization and clearance of pathogen). Accordingly, the immune response induced via mucosal (e.g., IN) administration of an immunogenic composition and the immune response induced via parenteral (e.g., IM) administration of the immunogenic composition are qualitatively different with respect to the total immunological response (e.g., comprising T-cell mediated components, cytokines, non-T-cell mediated components, etc.) of the organism to immunization via the two routes. For example, as shown by data collected during the development of embodiments of the technology provided herein, IN adiministration induces a Th17 response greater than the Th17 response induced by IM administration and IM adiministration induces a Th2 response greater than the Th2 response induced by IN administration. In some embodiments, IN administration induces a T cell mediated immune response not observed with IM administration.

As such, embodiments of the technology provided herein comprise methods, compositions, immunization regimens, and related technologies for inducing a multi-component immunogen-specific immune response in a subject. A used herein, a “component” of an immune response refers to a subset of the biological responses to immunogen that compose the (e.g., multi-component) immune response, e.g., comprising changes in antibody titers, cytokine profiles, T cell activities, etc. Some of the particular characteristics associated with one component may overlap in kind and/or amount (e.g., quantitatively and/or qualitatively) with the particular characteristics of another component. e.g., a first component comprising characteristic antibody titers, cytokine profiles, T cell activities, etc. and a second component comprising characteristic antibody titers, cytokine profiles, T cell activities, etc. may share some characteristics. In preferred embodiments, at least one characteristic (e.g., antibody titers, cytokine profiles, T cell activities, etc.) of a component of an immune response is different than the characteristics of a second component of an immune response. And, moreover, in preferred embodiments, at least one characteristic (e.g., antibody titers, cytokine profiles, T cell activities, etc.) of a component of an immune response is independent of another component and is not attainable by immunological phenomena (e.g., immunization via a particular route) that produce a second component of a multi-component immune response. Accordingly, a multi-component immunogen-specific immune response comprising at least two components provides an immune response that is different than the component immune responses associated with the individual components of the immune response.

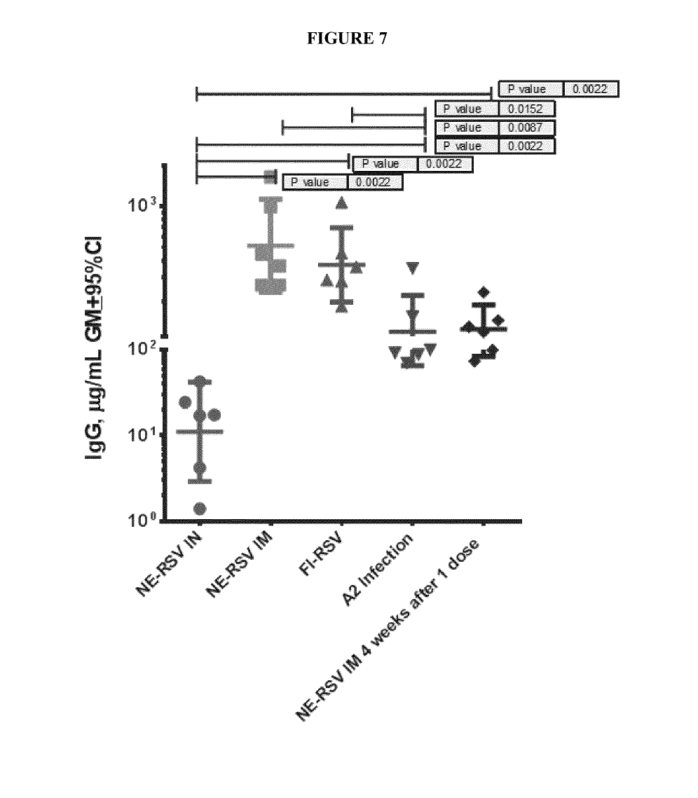

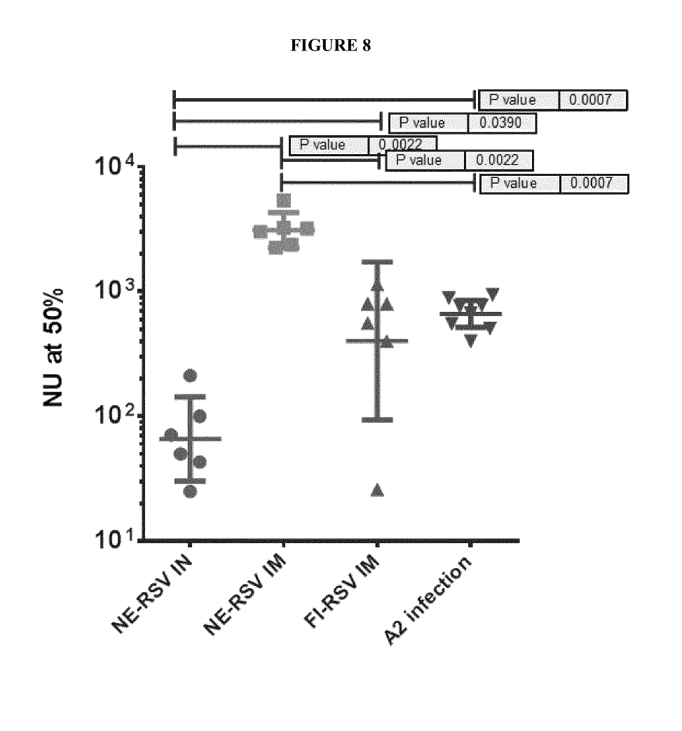

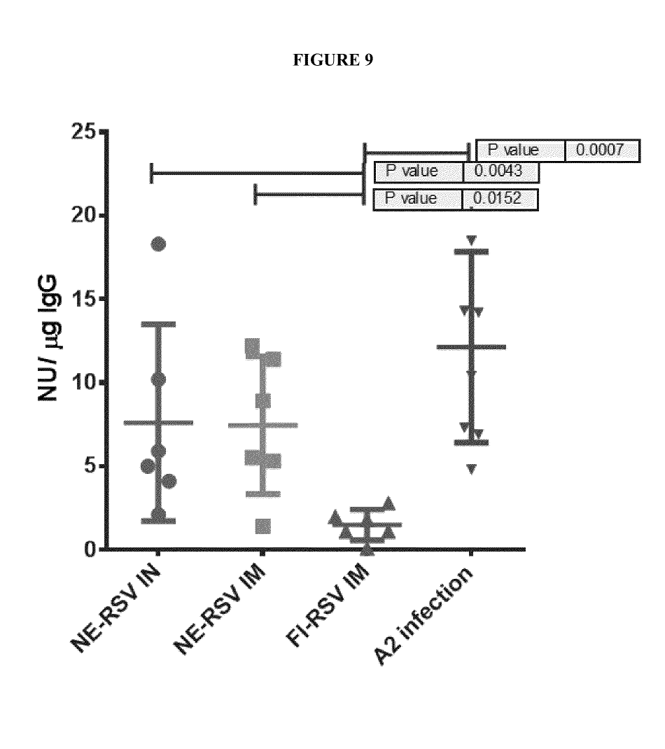

Furthermore, during the development of embodiments of the invention described, experiments were performed demonstrating that the serum antibodies produced by IN and IM immunization are functionally the same. As shown in FIG. 7, IM administration of an immunogenic composition induced a serum IgG antibody titer that was approximately 10 to 100 times the antibody titer in the serum induced by administration of the immunogenic composition via the IN route. In addition, three IM immunizations produced higher antibody titers in the serum (e.g., measured two weeks after the third immunization) than the antibody titer produced by one IM administration (e.g., measured 4 weeks after the single IM administration). The three IM immunizations also produced higher antibody titers in the serum (e.g., measured two weeks after the third immunization) than the antibody titers in the serum after immunization with formalin inactivated virus or infection with live virus (FIG. 7). The relative in vitro neutralization activities of sera from immunized animals (FIG. 8) closely resembled the trends observed in evaluating the antibody titers (FIG. 7). In particular, the neutralizing activity of serum from IM immunized animals was much higher (e.g., 10 to 100 times higher) than the neutralizing activity of serum from IN immunized animals (FIG. 8). Additionally, the neutralizing activity of serum from IM immunized animals was also higher (e.g., 2 to 5 times higher) than both the neutralizing activity of serum from animals immunized with formalin inactivated virus and the neutralizing activity of serum from animals infected with live virus (FIG. 8). After normalizing the neutralizing activities of sera from immunized animals for antibody (IgG) amount, the specific neutralization activities of the sera produced by IN and IM immunizations were similar or the same (FIG. 9); the specific neutralization activities of the sera produced by IN and IM immunizations were different than the specific activity of sera from animals immunized by formalin inactivated virus and the specific activity of sera from animals infected by live virus (FIG. 9). These data demonstrate that the serum antibodies produced by IN and IM immunization are functionally the same.

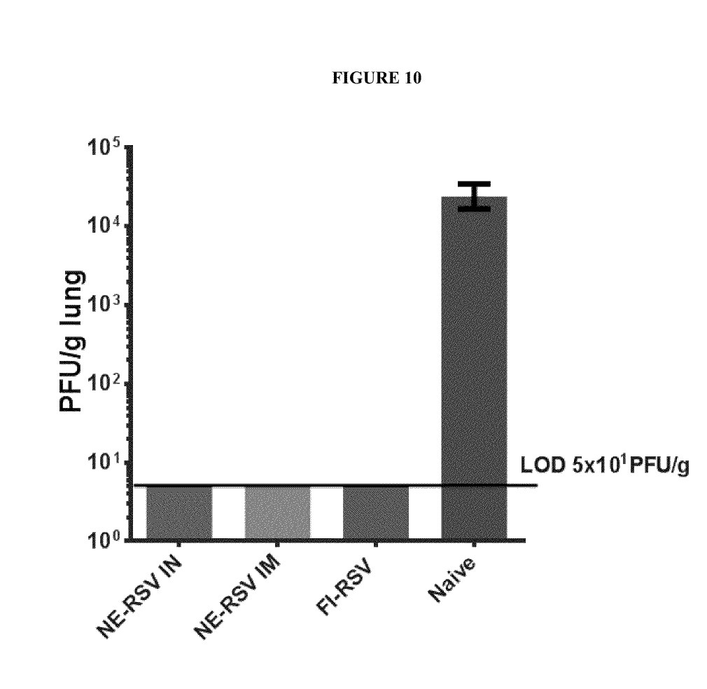

As production of antibodies (e.g., as measured by serum antibody titer) is a principal measure of the degree of immune protection conferred by an immune response (e.g., the result of an immunization), these data taken alone indicate that immunization via a mucosal (e.g., IN) route would have been predicted to provide less protection against infection than immunization via a parenteral (e.g., IM) route. However, additional experiments conducted during the development of embodiments of the invention provided herein demonstrated that both IN and IM immunization provided for a robust and complete clearance of a viral challenge in vivo (FIG. 10). That is, even though the antibody titers and serum neutralization activity of IN immunized animals was 1/100 to 1/10 of the antibody titers and serum neutralization activity of IM immunized animals, both IN and IM immunizations produced immunological protection in the mammalian (rat) animal model.

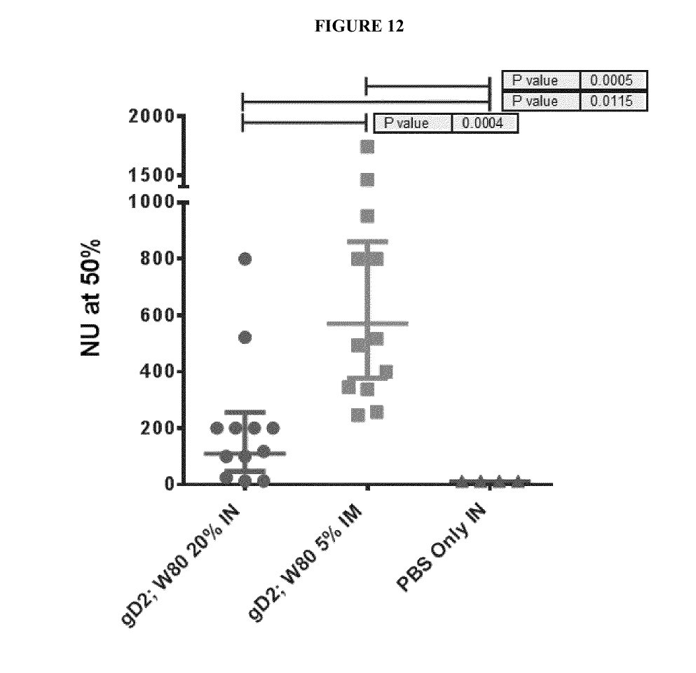

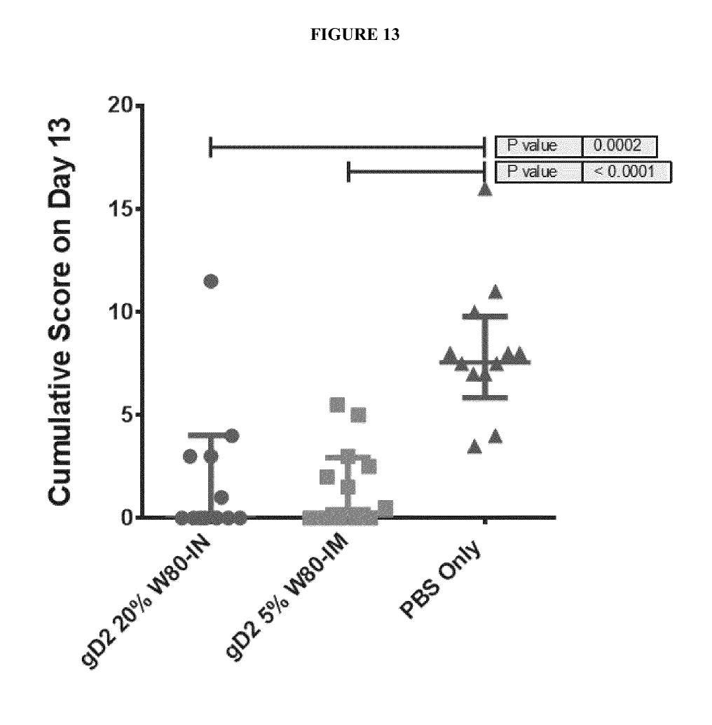

These results are further supported by in vitro studies in a guinea pig model (FIGS. 12-14). Data collected during the development of embodiments of the invention showed that protection against viral infection in IN and IM immunized animals was the same (FIG. 13) despite the IM immunization of guinea pigs having produced serum with a 6-fold higher neutralizing activity than serum from guinea pigs immunized by the IN route (FIG. 12).

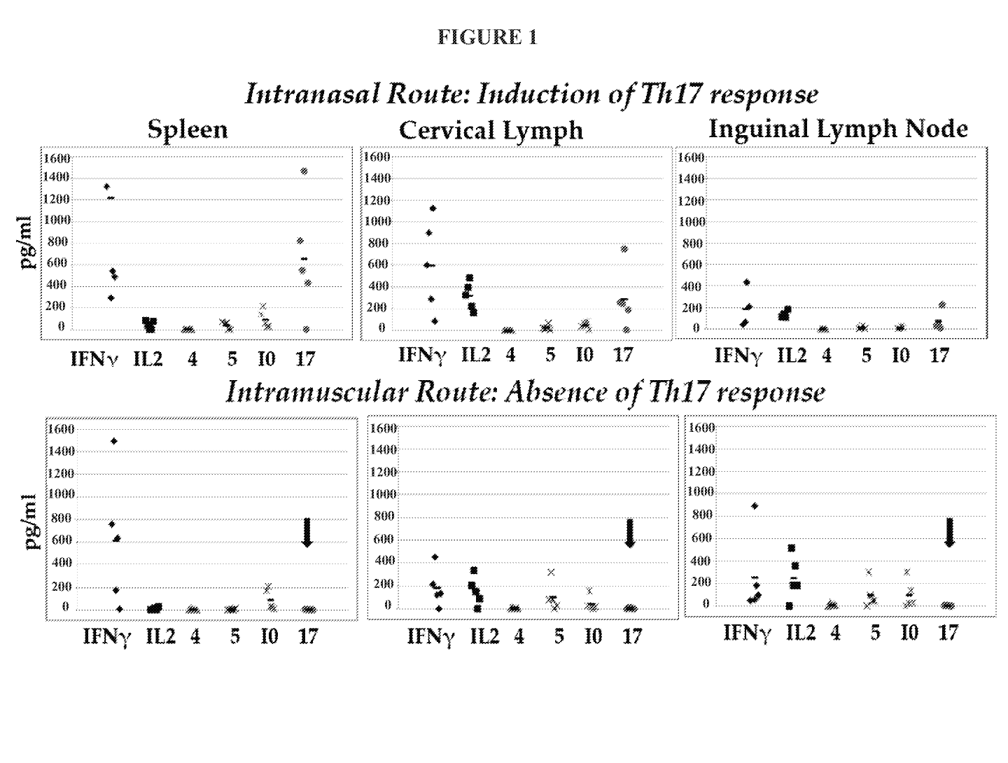

Without being bound to any theory (an understanding of the mechanism is not necessary to practice the present invention, and the present invention is not limited to any particular mechanism), it is proposed that IN immunization and IM immunization produce different immunological protective effects, e.g., induce different immunological responses and/or immune system components as part of a total immunological response to antigen. Indeed, the data collected during the development of embodiments of the technology support this mechanism. In particular, the data shown in FIGS. 1-6 demonstrate the different T-cell (e.g., Th17, Th1, Th2) and cytokine (e.g., IFN-gamma, IL-2, IL-4, IL-5, IL-10, IL-17, etc.) responses induced by IN versus IM immunization.

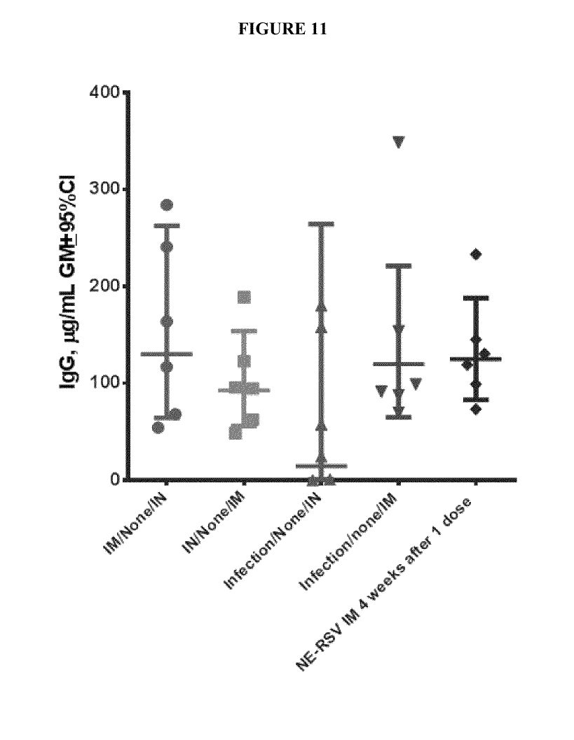

Furthermore, data collected during the development of embodiments of the invention demonstrated that the immunological routes or systems responsible for inducing immune responses to IN administration versus IM administration of an immunological composition are independent of one another. As shown in FIG. 11, a priming immunization administered IN is not boosted by a subsequent (e.g., given 12 weeks later) immunization administered IM. Also, a priming immunization administered IM is not boosted by a subsequent (e.g., given 12 weeks later) immunization administered IN. In fact, the immune response produced by a first IN administration followed by a subsequent IM administration produced an immune response similar to a single IM administration of the immunological composition to a naive animal (FIG. 11; FIG. 7). A boosting effect was only seen when prime and boost immunizations were administered via the same route. In particular, three IM administrations (FIG. 7) produced a higher (e.g., 10 times higher) antibody (e.g., IgG) titer than any of the dosing protocols in which an IN dose was followed by an IM dose or in which an IM dose was followed by an IN dose. The inability of IN administration to boost a previous IM administration and the inability of an IM administration to boost a previous IN administration demonstrates the independence of the two immunological pathways, systems, components (e.g., cytokine profiles, T-cell activity profiles, etc.), and/or mechanisms associated with the mucosal (e.g., IN) and parenteral (e.g., IM) immunization routes.

If immunological memory from the first IN exposure was accessible to the later IM immunization to provide a boost in immunity, then one would have predicted that the data from the “IN/none/IM” experiment (FIG. 11) would appear similar to the data from multiple IM immunizations (see, e.g., FIG. 7, “NE-RSV IM”). This outcome was not observed and the absence of the predicted effect confirms that the IN immunological pathway and the IM immunological pathway are wholly or nearly independent. Accordingly, administration of a vaccine via at least two routes provides a total immune response involving the independent and complementary aspects of both the mucosal immune response and the systemic immune response that provides an immunological protocol for vaccination that provides a different immune protection than a one-route immunization.

Furthermore, the data show that this robust immune response from dual-route immunization is the same or similar in both immunization comprising administering an immunogenic composition comprising a whole virus (e.g., see FIGS. 7-11) and in immunization comprising administering using an immunogenic composition comprising a recombinant peptide from a virus (e.g., see FIGS. 12-14).

As such, immunizing a subject (e.g., an animal such as a mammal, e.g., a human) by administration of an immunogenic composition via at least two different routes (e.g., mucosal and parenteral) induces two separate immunological responses that combine (e.g., additively and/or synergistically) to provide a robust total immune response to the antigen that is different than the immune response induced by administration of the immunogenic composition via one route alone.

In some embodiments, an addititive or synergistic effect is produced by administering to the subject an immunogenic composition comprising a nano emulsion and an immunogen via a first route and administering to the subject an immunogenic composition comprising a nano emulsion and an immunogen via a second route. In some embodiments, a subsequent booster immunization via the first route and/or a subsequent booster immunization via the second route (e.g, administering to the subject the immunogenic composition comprising a nano emulsion and an immunogen via the first route and/or administering to the subject an immunogenic composition comprising a nano emulsion and an immunogen via a second route) produces an additive or synergistic effect. In some embodiments, an addititive or synergistic effect is produced by administering to the subject an immunogenic composition comprising a nano emulsion and an immunogen via a first route and administering to the subject an immunogenic composition comprising a nano emulsion and an immunogen via a second route and, in addition, a boost immune response is produced by subsequently administering to the subject the immunogenic composition comprising a nano emulsion and an immunogen via the first route and/or subsequently administering to the subject an immunogenic composition comprising a nano emulsion and an immunogen via a second route. In some embodiments, both the initial administrations via the first and second routes and the subsequent administration(s) via the first and/or second routes produce an additive or synergistic effect. Accordingly, in some embodiments, a multi-component immune response is produced by administering to the subject an immunogenic composition comprising a nano emulsion and an immunogen at a first time via mucosal (e.g., IN and parenteral (e.g., IM) routes (e.g., an IN/IM administration) and subsequently administering to the subject an immunogenic composition comprising a nano emulsion and an immunogen at a second time via a mucosal (e.g., IN) and/or parenteral (e.g., IM) routes (e.g., first IN/IM+second IN, first IN/IM+second IM, first IN/IM+second IN/IM).

Accordingly, embodiments of the technology provide a method of inducing an immune response in a subject by administering an immunological composition by at least two routes (e.g., a parenteral, e.g., an IM, route and a mucosal, e.g., an IN, route), wherein the antibody titer produced by immunization via the first route (e.g., parenteral, e.g., IM, route) is higher (e.g., 2-fold, 5-fold, 10-fold, 100-fold higher) than the antibody titer produced by the second route (e.g., mucosal, e.g., IN, route). In some embodiments, the technology provides a method for inducing an immunogen-specific immune response in a subject, the method comprising administering to the subject via a first route an effective amount of an immunogenic composition comprising a nanoemulsion and an immunogen and administering to the subject via a second route an effective amount of an immunogenic composition comprising a nanoemulsion and an immunogen, wherein the systemic antibody titer produced in the subject by the administration via the first route is higher than the systemic antibody titer produced in the subject by the administration via the second route. In some embodiments, clearance of an infection from the subject by administration of an effective amount of the immunogenic composition via the first route alone is not significantly different than clearance of the infection from the subject by administration of an effective amount of the immunogenic composition via the second route alone. In some embodiments, the cytokine profile produced in the subject by administration of an effective amount of the immunogenic composition via the first route is different than the cytokine profile produced by administration of an effective amount of the immunogenic composition via the second route. In some embodiments, the T-cell response produced in the subject by administration of an effective amount of the immunogenic composition via the first route is different than the T-cell response produced by administration of an effective amount of the immunogenic composition via the second route. In some embodiments, the method induces an immune response in a subject that is different than either the immune response induced in the subject by administration of an effective amount of the immunogenic composition via the first route alone or the immune response induced in the subject by administration of an effective amount of the immunogenic composition via the second route alone. In some embodiments, administering to the subject via a first route an effective amount of an immunogenic composition does not prime administering to the subject via a second route an effective amount of an immunogenic composition or administering to the subject via a second route an effective amount of an immunogenic composition does not prime administering to the subject via a first route an effective amount of an immunogenic composition. And, in some embodiments, the systemic antibody titer or neutralizing activity induced in the subject by the method is not substantially different than either a systemic antibody titer or neutralizing activity induced in the subject by administration of an effective amount of the immunogenic composition via the first route alone or the systemic antibody titer or neutralizing activity induced in the subject by administration of an effective amount of the immunogenic composition via the second route alone; and the cytokine profile, T-cell response, or combined systemic and mucosal immunity induced in the subject by the method is different than the cytokine profile, T-cell response, or combined systemic and mucosal immunity induced in the subject by administration of an effective amount of the immunogenic composition via the first route alone and the cytokine profile, T-cell response, or combined systemic and mucosal immunity induced in the subject by administration of an effective amount of the immunogenic composition via the second route alone.

In some embodiments the administration by a first route and the administration by a second route are performed concurrently (e.g., within minutes or hours of each other and/or on the same day) and in some embodiments the administration by a first route and the administration by a second route are performed sequentially (e.g., separated by a time of 1, 2, 3, 4, 5, 6, 7, 8, 9, 10, 12, 12, 13, 14, 15, 16, 17, 18, 19, 20, 21, 22, 23, 24, 25, 26, 27, 28, 29, 30, 35, 40, 50, 55, 60, 65, 70, 75, 80, 85, 90, 95, 100, or more than 100 days; 1, 2, 3, 4, 5, 6, 7, 8, 9, 10, 11, or 12 months; 1, 2, 3, 4, 5, 6, 7, 8, 9, 10, 12, 12, 13, 14, 15, 16, 17, 18, 19, or 20 years). In some embodiments comprising sequential administration, a parenteral (e.g., IM) administration is first in time and in some embodiments a mucosal (e.g., IN) administration is first in time.

An effective amount of an immunogenic composition comprising nanoemulsion of the invention administered in a prime or boost delivery may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the vaccine. Where there is more than one administration of an immunogenic composition, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, or more, or combinations thereof. The invention is not limited to dosing intervals that are spaced equally in time, but encompass doses at non-equal intervals.

### I. Nanoemulsions as Anti-Pathogen Compositions

Nanoemulsion compositions utilized in some embodiments of the present invention have demonstrated anti-pathogen effect. For example, nanoemulsion compositions have been shown to inactivate bacteria (both vegetative and spore forms), virus, and fungi. In some embodiments of the present invention, pathogens are inactivated by exposure to nanoemulsions before being administered to a subject (e.g., to induce an immune response (e.g., for use as a vaccine)). Nanoemulsion adjuvant compositions can be used to rapidly inactivate bacteria. In certain embodiments, the compositions are particularly effective at inactivating Gram positive bacteria. In preferred embodiments, the inactivation of bacteria occurs after about five to ten minutes. Thus, bacteria may be contacted with an emulsion and will be inactivated in a rapid and efficient manner. It is expected that the period of time between the contacting and inactivation may be as little as 5-10 minutes where the bacteria is directly exposed to the emulsion. However, it is understood that when nanoemulsions are employed in a therapeutic context and applied systemically, the inactivation may occur over a longer period of time including, but not limited to, 5, 10, 15, 20, 25 30, 60 minutes post application. Further, in additional embodiments, inactivation may take two, three, four, five or six hours to occur.

Nanoemulsion adjuvants can also rapidly inactivate certain Gram negative bacteria for use in generating the vaccines of the present invention. In such methods, the bacteria inactivating emulsions are premixed with a compound that increases the interaction of the emulsion by the cell wall. The use of these enhancers in the vaccine compositions of the present invention is discussed herein below. It should be noted that certain emulsions (e.g., those comprising enhancers) are effective against certain Gram positive and negative bacteria.

Nanoemulsion adjuvants can also be utilized as anti-sporicidals. Without being bound to any theory (an understanding of the mechanism is not necessary to practice the present invention, and the present invention is not limited to any particular mechanism), it is proposed the that the sporicidal ability of these emulsions occurs through initiation of germination without complete reversion to the vegetative form leaving the spore susceptible to disruption by the emulsions. The initiation of germination could be mediated by the action of the emulsion or its components.

The bacteria-inactivating oil-in-water emulsions used in some embodiments of the present invention can be used to inactivate a variety of bacteria and bacterial spores upon contact. For example, the presently disclosed emulsions can be used to inactivate Bacillus including B. cereus, B. circulans and B. megatetium, also including Clostridium (e.g., C. botulinum and C. tetani). The nanoemulsions utilized in some embodiments of the present invention may be particularly useful in inactivating certain biological warfare agents (e.g., B. anthracis). In addition, the formulations of the present invention also find use in combating C. perfringens, H. influenzae, N. gonorrhoeae, S. agalactiae, S. pneumonia, S. pyogenes and V. cholerae classical and Eltor.

Nanoemulsion adjuvant compositions of the present invention have anti-viral properties.

Yet another property of the nanoemulsion adjuvants used in some embodiments of the present invention is that they possess antifungal activity. Common agents of fungal infections include various species of the genii Candida and Aspergillus, and types thereof, as well as others. While external fungus infections can be relatively minor, systemic fungal infections can give rise to serious medical consequences. There is an increasing incidence of fungal infections in humans, attributable in part to an increasing number of patients having impaired immune systems. Fungal disease, particularly when systemic, can be life threatening to patients having an impaired immune system.

### II. Nanoemulsion Adjuvant Compositions and Compositions for Inducing Immune Responses

In some embodiments, the present invention provides compositions for inducing immune responses comprising an immunogenic composition comprising nanoemulsion (e.g., independently and/or combined with one or more immunogens (e.g., inactivated pathogens or pathogen products)). A variety of nanoemulsion that find use in the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes).

Nanoemulsions (e.g., independently or combined with one or more immunogens (e.g., pathogens or pathogen products)) of the present invention may be combined in any suitable amount utilizing a variety of delivery methods. Any suitable pharmaceutical formulation may be utilized, including, but not limited to, those disclosed herein. Suitable formulations may be tested for immunogenicity using any suitable method. For example, in some embodiments, immunogenicity is investigated by quantitating both specific T-cell responses and antibody titer. Nanoemulsion compositions of the present invention may also be tested in animal models of infectious disease states. Suitable animal models, pathogens, and assays for immunogenicity include, but are not limited to, those described herein.

An immunogenic composition comprising nanoemulsion enables and enhances immune responses. Adjuvants have been traditionally developed from pro-inflammatory substances, such as a toxin or microbiological component, found to trigger signaling pathways and cytokine production (See, e.g., Graham, B. S., Plos Medicine, 2006. 3(1): p. e57). Also, enterotoxin-based adjuvants, such as cholera toxin, have been associated with inducing inflammation in the nasal mucosa and with production of the inflammatory cytokines and transport of the vaccine along olfactory neurons into the olfactory bulbs (See, e.g., van Ginkel, F. W., et al., Infect Immun., 2005. 73(10): p. 6892-6902). Some patients treated with a flu vaccine based on one of these toxins (NASALFLU, BERNA Biotech), developed Bell's palsy (See, e.g., Mutsch, M., et al., New England Journal of Medicine, 2004. 350(9): p. 896-903) presumably due to the transition of vaccine or vaccine components into the olfactory bulb. This finding led to NASALFLU being withdrawn. In contrast, in some embodiments, the present invention provides immunogenic compositions comprising nanoemulsion with no significant inflammation in animals and no evidence of the composition in the olfactory bulb. Thus the present invention provides, in some embodiments, compositions and methods for inducing immune responses (e.g., immunity to) to pathogens utilizing needle-free mucosal administration, induction of systemic immunity comparable with conventional vaccines, as well as mucosal and cellular immune responses that are not elicited by injected, non-nanoemulsion adjuvant-based (e.g., aluminum-based) vaccines (See, e.g., the Examples).

In some embodiments, the present invention provides methods of inducing an immune response and an immunogenic composition comprising nanoemulsion useful in such methods (e.g., a nanoemulsion adjuvant composition). In some embodiments, methods of inducing an immune response in a host subject provided by the present invention are used for vaccination. For example, in some embodiments, the present invention provides a composition comprising an immunogenic composition comprising nanoemulsion and one or a plurality of immunogens (e.g., derived from a plurality of pathogens (e.g., one or a plurality of pathogens inactivated by a nanoemulsion of the present invention and/or one or a plurality of protein and/or peptide antigens derived from (e.g., isolated and/or recombinantly produced from) one or a plurality of pathogens)); as well as methods of administering the composition (e.g., in a heterologous prime/boost protocol) to a subject under conditions such that the subject generates an immune response to the one or a plurality of pathogens and/or immunogens. Any prime/boost protocol described herein may be utilized. In some embodiments, inducing an immune response induces immunity to one or a plurality of immunogens in the subject. In some embodiments, inducing an immune response to the immunogens induces immunity to the plurality of pathogens from which the immunogens are derived. In some embodiments, immunity comprises systemic immunity. In some embodiments, immunity comprises mucosal immunity. In some embodiments, the immune response comprises a systemic IgG response to the immunogens (e.g., comparable to monovalent vaccine formulations). In some embodiments, the immune response comprises a mucosal IgA response to the immunogens.

Thus, as described herein, the present invention, in one embodiment, provides nanoemulsions useful for formulating immunogenic compositions, suitable to be used as, for example, vaccines. The immunogenic compositions described herein elicit an immune response by the host subject to which it is administered (e.g., including the production of cytokines and other immune factors). In some embodiments, an immunogenic composition comprising nanoemulsion is formulated to include at least one antigen. An antigen may be an inactivated pathogen or an antigenic fraction of a pathogen. The pathogen may be, for example, a virus, a bacterium or a parasite. The pathogen may be inactivated by a chemical agent, such as formaldehyde, glutaraldehyde, beta-propiolactone, ethyleneimine and derivatives, the nanoemulsion adjuvant itself, or other compounds. The pathogen may also be inactivated by a physical agent, such as UV radiation, gamma radiation, “heat shock” and X-ray radiation. An antigenic fraction of a pathogen can be produced by means of chemical or physical decomposition methods, followed, if desired, by separation of a fraction by means of chromatography, centrifugation and similar techniques. Alternatively, antigens or haptens can be prepared by means of organic synthetic methods, or, in the case of, for example, polypeptides and proteins, by means of recombinant DNA methods. In some embodiments, an adjuvant composition of the invention is co-administered with a vaccine available in the marketplace (e.g., in order to generate a more robust immune response, in order to skew the immune response (e.g., toward a Th1 and away from a Th2 response) or to balance the type of immune response elicited by the vaccine).

In some embodiments, the present invention provides a method of inducing an immune response in a subject comprising administering to a subject an immunogenic composition comprising nanoemulsion under conditions such that the expression of one or more genes associated with an immune response (e.g., a Th1 type immune response, a Th2 type immune response, and/or a Th17 immune response) is altered (e.g., enhances or reduced) in the subject (e.g., within dendritic cells). In some embodiments, the present invention provides nanoemulsion adjuvant compositions that stimulate and/or elicit immune responses (e.g., innate immune responses) when administered to a subject (e.g., a human subject)).

Host innate immune responses enable the host to differentiate self from pathogen and provide a rapid inflammatory response, including production of cytokines and chemokines, elaboration of effector molecules, such as NO, and interactions with the adaptive immune response (See, e.g., Janeway and Medzhitov, (2002) Annu Rev. Immunol. 20, 197-216). Molecular understanding of innate immunity in humans evolved the mid-1990s when the Drosophila protein Toll was shown to be critical for defending flies against fungal infections (See, e.g., Lemaitre et al., (1996). Cell 86, 973-983). The human Toll-like receptor (TLR) family includes at least ten receptors that play important roles in innate immunity (See, e.g., Akira et al., (2006) Cell 124, 783-801; Beutler et al., (2006) Annu. Rev. Immunol. 24, 353-380; and Takeda et al., (2003). Annu Rev. Immunol. 21, 335-376).

In general, TLRs recognize and respond to diverse microbial molecules and enable the innate immune system to discriminate among groups of pathogens and to induce an appropriate cascade of effector responses. Individual TLRs recognize a distinct repertoire of conserved molecules (e.g., microbial products). For example, well-characterized receptor-ligand pairs include TLR4 and LPS (lipopolysaccharide), TLR5 and flagellin, TLR1/TLR2/TLR6 and lipoproteins, and TLR3/TLR7/TLR8/TLR9 and different nucleic acid motifs. Collectively, the family of TLRs allows a host's innate immune system to detect the presence of foreign molecules (e.g., microbial products of most microbial pathogens or other substances).

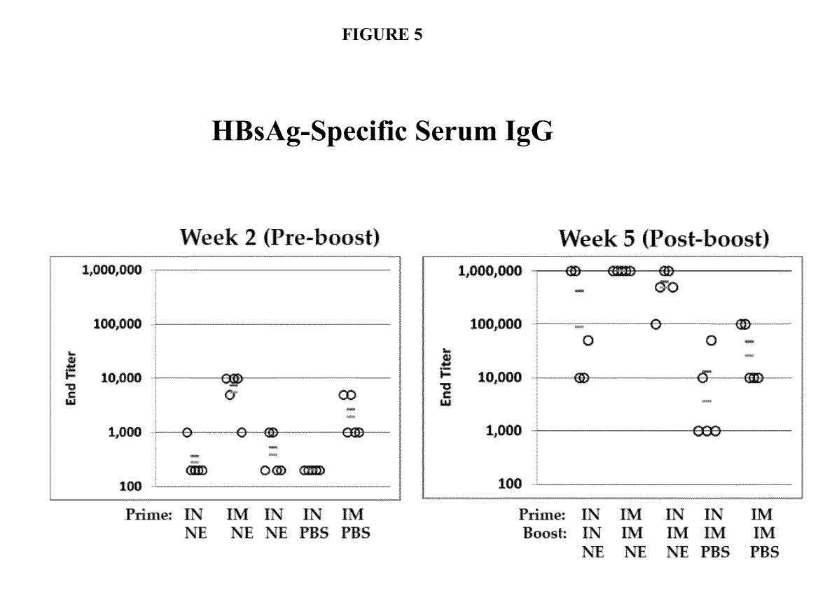

TLRs are classified as members of the IL-1R (IL-1 receptor) superfamily on the basis of a shared cytoplasmic region known as the TIR (Toll/IL-1R) domain. The extracellular portions of TLRs are rather diverse, comprising varying numbers of leucine-rich repeats. Following encounter with a microbe, TLRs trigger a complex cascade of events that lead to the induction of a range of proinflammatory genes (See, e.g., Yamamoto et al., (2002) Nature 420, 324-329 (See, e.g., Misch and Hawn, Clin Sci 2008, 114, 347-360, and also FIG. 5)). Ligand binding results in the recruitment of several molecules to the receptor complex. These include TIR-domain-containing adaptor molecules such as MyD88 (myeloid differentiation primary response gene 88), TIRAP/Mal (TIR-domain-containing adapter/MyD88 adaptor-like), TICAM1/TRIF (TIR-domain-containing adaptor molecule 1/TIR-domain-containing adaptor-inducing interferon b) and TRAM (TRIF-related adaptor molecule). Further recruitment of molecules includes IRAKs (IL-1R-associated kinases (IRAK1, 2, 3 (M) and 4)) as well as TRAF6 (TNF receptor-associated factor 6). IRAK1 and TRAF6 then dissociate and bind another complex that comprises TAK1 (TGF (transforming growth factor)-b-activated kinase 1) and TAB1, 2 and 3 (TAK-1-binding proteins 1, 2 and 3). TAK1 then activates IKK (IkB (inhibitor of NF-kB (nuclear factor kB)) kinase). The activity of this complex is regulated by IKKg (also known as NEMO (NF-kB essential modulator)). IKK-mediated phosphorylation of IkB leads to its degradation, allowing NF-kB to translocate to the nucleus and promote the transcription of multiple proinflammatory genes, including TNF, IL-1b and IL-6.

TLR activation by pathogens, or by molecules derived therefrom, induces intracellular signaling that primarily results in activation of the transcription factor NF-kB (See, e.g., Beg, 2002, Trends Immunol. 2002 23 509-12.) and modulation of cytokine production. However, a series of other pathways can also be triggered, including p38 mitogen activated kinase, c-Jun-N-terminal kinase and extracellular signal related kinase pathways (See, e.g., Flohe, et al., 2003, J Immunol, 170 2340-2348; Triantafilou & Triantafilou, 2002, Trends Immunol, 23 301-304). The patterns of gene expression induced by ligation of the different TLRs are distinct but often overlap. For instance a large proportion of the genes upregulated by TLR3 agonists and double stranded RNA are also upregulated by TLR4 agonists and LPS (See, e.g., Doyle et al., 2002, Immunity, 17 251-263). TLR4 activation by LPS in macrophages results in TNF-α, IL-12 IL-1β, RANTES and MIP1β secretion (See, e.g., Flohe et al., supra; Jones et al., 2002, J Leukoc Biol, 69 1036-1044).

Nanoemulsion compositions may be administered before, after or co-administered with compositions comprising one or more antigens. In some embodiments, a nanoemulsion is administered to a subject prior to (e.g., minutes, hours, days before) the subject being administered a composition comprising an antigen (e.g., a killed pathogen (e.g., virus, bacteria, or other pathogen described herein) or pathogen component) (e.g., so as to prime the subject's immune system to respond to the antigen and produce a desired immune response against the same). In some embodiments, a nanoemulsion is administered to a subject after (e.g., minutes, hours, days after) the subject is administered a composition comprising an antigen (e.g., a killed pathogen (e.g., virus, bacteria, or other pathogen described herein) or pathogen component) (e.g., so as to boost and/or skew the subject's immune system to respond to the antigen and produce a desired immune response against the same). In some embodiments, a nanoemulsion is administered to a subject concurrent with (e.g., co-administered to) the subject being administered a composition comprising an antigen (e.g., a killed pathogen (e.g., virus, bacteria, or other pathogen described herein) or pathogen component) (e.g., so as to prime the subject's immune system to respond to the antigen and produce a desired immune response against the same).

In some embodiments, the present invention provides immunogenic compositions comprising nanoemulsion that generate a desired immune response in a subject administered the composition (e.g., an adaptive immune response). For example, in some embodiments, the present invention provides immunogenic compositions comprising nanoemulsion that skew a host's immune response, when combined with and/or mixed with one or a plurality of antigens, away from Th2 type immune response and toward a Th1 type immune response. In particular, conventional alum based vaccines for a variety of diseases such as respiratory syncitial virus (RSV), anthrax, and hepatitis B virus each lead to a predominant Th2 type immune response in a subject administered the vaccine (e.g., characterized by enhanced expression of Th2 type cytokines and the production of IgG1 antibodies). However, immunogenic compositions (e.g., vaccines) produced with nanoemulsion compositions of the invention are able to redirect the conventionally observed Th2 type immune response in host subjects administered conventional vaccines. Immunogenic compositions comprising an immunogenic composition comprising nanoemulsion of the invention can likewise be utilized to skew a host immune response against hepatitis B virus away from a Th2 type immune response and toward a Th1 type immune response.

Thus, in some embodiments, the present invention provides compositions and methods for skewing and/or redirecting a host's immune response (e.g., away from Th2 type immune responses and toward Th1 type immune responses) to one or a plurality of immunogens/antigens. In some embodiments, skewing and/or redirecting a host's immune response (e.g., away from Th2 type immune responses and toward Th1 type immune responses) to one or a plurality of immunogens/antigens comprises providing one or more antigens (e.g., recombinant antigens, isolated and/or purified antigens, and/or killed whole pathogens) that are historically associated with generation of a Th2 type immune response when administered to a subject (e.g., RSV antigen, hepatitis B virus antigen, etc.), combining the one or more antigens with a nanoemulsion of the invention, and administering the nanoemulsion-antigen mixture to a subject under conditions (e.g., via a prime/boost protocol) sufficient to induce the desired immune response.

In some embodiments, the present invention provides immunogenic compositions comprising nanoemulsion that reduce the number of booster injections (e.g., of an antigen containing composition) required to achieve protection. In some embodiments, the present invention provides an immunogenic composition comprising nanoemulsion and administration thereof (e.g., via a heterologous prime/boost protocol) that result in a higher proportion of recipients achieving seroconversion. In some embodiments, the present invention provides immunogenic compositions comprising nanoemulsion that are useful for selectively skewing adaptive immunity toward Th1, Th2, or cytotoxic T cell responses (e.g., allowing effective immunization by distinct routes (e.g., such as via the skin or mucosa)). In some embodiments, the present invention provides immunogenic compositions comprising nanoemulsion that elicit optimal responses in subjects in which most contemporary vaccination strategies are not optimally effective (e.g., in very young and/or very old populations). In some embodiments, the present invention provides immunogenic compositions comprising nanoemulsion that provide efficacy and safety needed for vaccination regimens that involve different delivery routes and elicitation of distinct types of immunity. In some embodiments, the present invention provides nanoemulsion compositions that stimulate antibody responses and have little toxicity and that can be utilized with a range of antigens for which they provide adjuvanticity and the types of immune responses they elicit. In some embodiments, the present invention provides immunogenic compositions comprising nanoemulsion that meet global supply requirements (e.g., in response to a pathogenic (e.g., influenza) pandemic).

### Generation of Antibodies

An immunogenic composition comprising a nanoemulsion (e.g., independently or together with an antigen) can be used to immunize a mammal, such as a mouse, rat, rabbit, guinea pig, monkey, or human, to produce polyclonal antibodies. If desired, an antigen can be conjugated to a carrier protein, such as bovine serum albumin, thyroglobulin, keyhole limpet hemocyanin or other carrier described herein. Depending on the host species, various additional adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, nanoemulsions described herein, keyhole limpet hemocyanin, and dinitrophenol). Among adjuvants used in humans, BCG (bacilli Calmette-Guerin) and Corynebacterium parvum are especially useful.

Monoclonal antibodies can be prepared using any technique which provides for the production of antibody molecules by continuous cell lines in culture. These techniques include, but are not limited to, the hybridoma technique, the human B cell hybridoma technique, and the EBV hybridoma technique (See, e.g., Kohler et al., Nature 256, 495 497, 1985; Kozbor et al., J. Immunol. Methods 81, 3142, 1985; Cote et al., Proc. Natl. Acad. Sci. 80, 2026 2030, 1983; Cole et al., Mol. Cell. Biol. 62, 109 120, 1984).

In addition, techniques developed for the production of “chimeric antibodies,” the splicing of mouse antibody genes to human antibody genes to obtain a molecule with appropriate antigen specificity and biological activity, can be used (See, e.g., Morrison et al., Proc. Natl. Acad. Sci. 81, 68516855, 1984; Neuberger et al., Nature 312, 604 608, 1984; Takeda et al., Nature 314, 452 454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (See, e.g., Burton, Proc. Natl. Acad. Sci. 88, 11120 23, 1991).

Single-chain antibodies also can be constructed using a DNA amplification method, such as PCR, using hybridoma cDNA as a template (See, e.g., Thirion et al., 1996, Eur. J. Cancer Prey. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught, for example, in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (See, e.g., Verhaar et al., 1995, Int. J. Cancer 61, 497-501; Nicholls et al., 1993, J. Immunol. Meth. 165, 81-91).

Antibodies can be produced by inducing in vivo production in the lymphocyte population or by screening immunoglobulin libraries or panels of highly specific binding reagents as disclosed in the literature (See, e.g., Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared. Antibodies can be purified by methods well known in the art. For example, antibodies can be affinity purified by passage over a column to which the relevant antigen is bound. The bound antibodies can then be eluted from the column using a buffer with a high salt concentration.

### Nanoemulsions

The present invention is not limited by the type of nanoemulsion adjuvant utilized (e.g., in a heterologous prime/boost regimen). Indeed, a variety of nanoemulsions are contemplated to be useful in the present invention.

For example, in some embodiments, a nanoemulsion comprises (i) an aqueous phase; (ii) an oil phase; and at least one additional compound. In some embodiments of the present invention, these additional compounds are admixed into either the aqueous or oil phases of the composition. In other embodiments, these additional compounds are admixed into a composition of previously emulsified oil and aqueous phases. In certain of these embodiments, one or more additional compounds are admixed into an existing emulsion composition immediately prior to its use. In other embodiments, one or more additional compounds are admixed into an existing emulsion composition prior to the compositions immediate use.

Additional compounds suitable for use in a nanoemulsion of the present invention include, but are not limited to, one or more organic, and more particularly, organic phosphate based solvents, surfactants and detergents, cationic halogen containing compounds, germination enhancers, interaction enhancers, food additives (e.g., flavorings, sweeteners, bulking agents, and the like) and pharmaceutically acceptable compounds (e.g., carriers). Certain exemplary embodiments of the various compounds contemplated for use in the compositions of the present invention are presented below. Unless described otherwise, nanoemulsions are described in undiluted form.

Nanoemulsion adjuvant compositions of the present invention are not limited to any particular nanoemulsion. Any number of suitable nanoemulsion compositions may be utilized in the vaccine compositions of the present invention, including, but not limited to, those disclosed in Hamouda et al., J. Infect Dis., 180:1939 (1999); Hamouda and Baker, J. Appl. Microbiol., 89:397 (2000); and Donovan et al., Antivir. Chem. Chemother., 11:41 (2000). Preferred nanoemulsions of the present invention are those that are non-toxic to animals. In preferred embodiments, nanoemulsions utilized in the methods of the present invention are stable, and do not decompose even after long storage periods (e.g., one or more years). Additionally, preferred emulsions maintain stability even after exposure to high temperature and freezing. This is especially useful if they are to be applied in extreme conditions (e.g., extreme heat or cold).

Some embodiments of the present invention employ an oil phase containing ethanol. For example, in some embodiments, the emulsions of the present invention contain (i) an aqueous phase and (ii) an oil phase containing ethanol as the organic solvent and optionally a germination enhancer, and (iii) TYLOXAPOL as the surfactant (preferably 2-5%, more preferably 3%). This formulation is highly efficacious for inactivation of pathogens and is also non-irritating and non-toxic to mammalian subjects (e.g., and thus can be used for administration to a mucosal surface).

In some other embodiments, the emulsions of the present invention comprise a first emulsion emulsified within a second emulsion, wherein (a) the first emulsion comprises (i) an aqueous phase; and (ii) an oil phase comprising an oil and an organic solvent; and (iii) a surfactant; and (b) the second emulsion comprises (i) an aqueous phase; and (ii) an oil phase comprising an oil and a cationic containing compound; and (iii) a surfactant.

### Exemplary Formulations

The following description provides a number of exemplary emulsions including formulations for compositions BCTP and X₈W₆₀PC. BCTP comprises a water-in oil nanoemulsion, in which the oil phase was made from soybean oil, tri-n-butyl phosphate, and TRITON X-100 in 80% water. X₈W₆₀PC comprises a mixture of equal volumes of BCTP with W₈₀8P. W₈₀8P is a liposome-like compound made of glycerol monostearate, refined oya sterols (e.g., GENEROL sterols), TWEEN 60, soybean oil, a cationic ion halogen-containing CPC and peppermint oil. The GENEROL family are a group of a polyethoxylated soya sterols (Henkel Corporation, Ambler, Pa.). Exemplary emulsion formulations useful in the present invention are provided in Table 1. These particular formulations may be found in U.S. Pat. No. 5,700,679 (NN); U.S. Pat. Nos. 5,618,840; 5,549,901 (W₈₀8P); and U.S. Pat. No. 5,547,677, each of which is hereby incorporated by reference in their entireties. Certain other emulsion formulations are presented U.S. patent application Ser. No. 10/669,865, hereby incorporated by reference in its entirety.

The X₈W₆₀PC emulsion is manufactured by first making the W₈₀8P emulsion and BCTP emulsions separately. A mixture of these two emulsions is then re-emulsified to produce a fresh emulsion composition termed X₈W₆₀PC. Methods of producing such emulsions are described in U.S. Pat. Nos. 5,103,497 and 4,895,452 (each of which is herein incorporated by reference in their entireties).

TABLE 1

| | | Water to Oil |
|---|---|---|
| | Oil Phase Formula | Phase Ratio (Vol/Vol) |
| BCTP | 1 vol. Tri(N-butyl)phosphate | 4:1 |
| | 1 vol. TRITON X-100 | |
| | 8 vol. Soybean oil | |
| NN | 86.5 g Glycerol monooleate | 3:1 |
| | 60.1 ml Nonoxynol-9 | |
| | 24.2 g GENEROL 122 | |
| | 3.27 g Cetylpyridinium chloride | |
| | 554 g Soybean oil | |
| W₈₀8P | 86.5 g Glycerol monooleate | 3.2:1 |
| | 21.2 g Polysorbate 60 | |
| | 24.2 g GENEROL 122 | |
| | 3.27 g Cetylpyddinium chloride | |
| | 4 ml Peppermint oil | |
| | 554 g Soybean oil | |
| SS | 86.5 g Glycerol monooleate | 3.2:1 |
| | 21.2 g Polysorbate 60 | (1% bismuth in water) |
| | 24.2 g GENEROL 122 | |
| | 3.27 g Cetylpyridinium chloride | |
| | 554 g Soybean oil | |

The compositions listed above are only exemplary and those of skill in the art will be able to alter the amounts of the components to arrive at a nanoemulsion composition suitable for the purposes of the present invention. Those skilled in the art will understand that the ratio of oil phase to water as well as the individual oil carrier, surfactant CPC and organic phosphate buffer, components of each composition may vary.

Although certain compositions comprising BCTP have a water to oil ratio of 4:1, it is understood that the BCTP may be formulated to have more or less of a water phase. For example, in some embodiments, there is 3, 4, 5, 6, 7, 8, 9, 10, or more parts of the water phase to each part of the oil phase. The same holds true for the W₈₀8P formulation. Similarly, the ratio of Tri (N-butyl) phosphate:TRITON X-100:soybean oil also may be varied.

Although Table 1 lists specific amounts of glycerol monooleate, polysorbate 60, GENEROL 122, cetylpyridinium chloride, and carrier oil for W₈₀8P, these are merely exemplary. An emulsion that has the properties of W₈₀8P may be formulated that has different concentrations of each of these components or indeed different components that will fulfill the same function. For example, the emulsion may have between about 80 to about 100 g of glycerol monooleate in the initial oil phase. In other embodiments, the emulsion may have between about 15 to about 30 g polysorbate 60 in the initial oil phase. In yet another embodiment the composition may comprise between about 20 to about 30 g of a GENEROL sterol, in the initial oil phase.

Individual components of nanoemulsions (e.g. in an immunogenic composition of the present invention) can function both to inactivate a pathogen as well as to contribute to the non-toxicity of the emulsions. For example, the active component in BCTP, TRITON-X100, shows less ability to inactivate a virus at concentrations equivalent to 11% BCTP. Adding the oil phase to the detergent and solvent markedly reduces the toxicity of these agents in tissue culture at the same concentrations. While not being bound to any theory (an understanding of the mechanism is not necessary to practice the present invention, and the present invention is not limited to any particular mechanism), it is suggested that the nanoemulsion enhances the interaction of its components with the pathogens thereby facilitating the inactivation of the pathogen and reducing the toxicity of the individual components. Furthermore, when all the components of BCTP are combined in one composition but are not in a nanoemulsion structure, the mixture is not as effective at inactivating a pathogen as when the components are in a nanoemulsion structure.

Numerous additional embodiments presented in classes of formulations with like compositions are presented below. The following compositions recite various ratios and mixtures of active components. One skilled in the art will appreciate that the below recited formulation are exemplary and that additional formulations comprising similar percent ranges of the recited components are within the scope of the present invention.

In certain embodiments of the present invention, a nanoemulsion comprises from about 3 to 8 vol. % of TYLOXAPOL, about 8 vol. % of ethanol, about 1 vol. % of cetylpyridinium chloride (CPC), about 60 to 70 vol. % oil (e.g., soybean oil), about 15 to 25 vol. % of aqueous phase (e.g., DiH₂O or PBS), and in some formulations less than about 1 vol. % of 1N NaOH. Some of these embodiments comprise PBS. It is contemplated that the addition of 1N NaOH and/or PBS in some of these embodiments, allows the user to advantageously control the pH of the formulations, such that pH ranges from about 7.0 to about 9.0, and more preferably from about 7.1 to 8.5 are achieved. For example, one embodiment of the present invention comprises about 3 vol. % of TYLOXAPOL, about 8 vol. % of ethanol, about 1 vol. % of CPC, about 64 vol. % of soybean oil, and about 24 vol. % of DiH₂O (designated herein as Y3EC). Another similar embodiment comprises about 3.5 vol. % of TYLOXAPOL, about 8 vol. % of ethanol, and about 1 vol. % of CPC, about 64 vol. % of soybean oil, and about 23.5 vol. % of DiH₂O (designated herein as Y3.5EC). Yet another embodiment comprises about 3 vol. % of TYLOXAPOL, about 8 vol. % of ethanol, about 1 vol. % of CPC, about 0.067 vol. % of 1N NaOH, such that the pH of the formulation is about 7.1, about 64 vol. % of soybean oil, and about 23.93 vol. % of DiH₂O (designated herein as Y3EC pH 7.1). Still another embodiment comprises about 3 vol. % of TYLOXAPOL, about 8 vol. % of ethanol, about 1 vol. % of CPC, about 0.67 vol. % of 1N NaOH, such that the pH of the formulation is about 8.5, and about 64 vol. % of soybean oil, and about 23.33 vol. % of DiH₂O (designated herein as Y3EC pH 8.5). Another similar embodiment comprises about 4% TYLOXAPOL, about 8 vol. % ethanol, about 1% CPC, and about 64 vol. % of soybean oil, and about 23 vol. % of DiH₂O (designated herein as Y4EC). In still another embodiment the formulation comprises about 8% TYLOXAPOL, about 8% ethanol, about 1 vol. % of CPC, and about 64 vol. % of soybean oil, and about 19 vol. % of DiH₂O (designated herein as Y8EC). A further embodiment comprises about 8 vol. % of TYLOXAPOL, about 8 vol. % of ethanol, about 1 vol. % of CPC, about 64 vol. % of soybean oil, and about 19 vol. % of 1×PBS (designated herein as Y8EC PBS).

In some embodiments of the present invention, a nanoemulsion comprises about 8 vol. % of ethanol, and about 1 vol. % of CPC, and about 64 vol. % of oil (e.g., soybean oil), and about 27 vol. % of aqueous phase (e.g., DiH₂O or PBS) (designated herein as EC).

In some embodiments, a nanoemulsion comprises from about 8 vol. % of sodium dodecyl sulfate (SDS), about 8 vol. % of tributyl phosphate (TBP), and about 64 vol. % of oil (e.g., soybean oil), and about 20 vol. % of aqueous phase (e.g., DiH₂O or PBS) (designated herein as S8P).

In some embodiments, a nanoemulsion comprises from about 1 to 2 vol. % of TRITON X-100, from about 1 to 2 vol. % of TYLOXAPOL, from about 7 to 8 vol. % of ethanol, about 1 vol. % of cetylpyridinium chloride (CPC), about 64 to 57.6 vol. % of oil (e.g., soybean oil), and about 23 vol. % of aqueous phase (e.g., DiH₂O or PBS). Additionally, some of these formulations further comprise about 5 mM of L-alanine/Inosine, and about 10 mM ammonium chloride. Some of these formulations comprise PBS. It is contemplated that the addition of PBS in some of these embodiments, allows the user to advantageously control the pH of the formulations. For example, one embodiment of the present invention comprises about 2 vol. % of TRITON X-100, about 2 vol. % of TYLOXAPOL, about 8 vol. % of ethanol, about 1 vol. % CPC, about 64 vol. % of soybean oil, and about 23 vol. % of aqueous phase DiH₂O. In another embodiment the formulation comprises about 1.8 vol. % of TRITON X-100, about 1.8 vol. % of TYLOXAPOL, about 7.2 vol. % of ethanol, about 0.9 vol. % of CPC, about 5 mM L-alanine/Inosine, and about 10 mM ammonium chloride, about 57.6 vol. % of soybean oil, and the remainder of 1×PBS (designated herein as 90% X2Y2EC/GE).

In alternative embodiments, a nanoemulsion comprises from about 5 vol. % of TWEEN 80, from about 8 vol. % of ethanol, from about 1 vol. % of CPC, about 64 vol. % of oil (e.g., soybean oil), and about 22 vol. % of DiH₂O (designated herein as W₈₀5EC). In yet another alternative embodiment, a nanoemulsion comprises from about 5 vol. % of TWEEN 80, from about 8 vol. % of ethanol, about 64 vol. % of oil (e.g., soybean oil), and about 23 vol. % of DiH₂O (designated herein as W₈₀5E).

In some embodiments, the present invention provides a nanoemulsion comprising from about 5 vol. % of Poloxamer-407, from about 8 vol. % of ethanol, from about 1 vol. % of CPC, about 64 vol. % of oil (e.g., soybean oil), and about 22 vol. % of DiH₂O (designated herein as P₄₀₇5EC). Although an understanding of the mechanism is not necessary to practice the present invention, and the present invention is not limited to any particular mechanism, in some embodiments, a nanoemulsion comprising Poloxamer-407 does not elicit and/or augment immune responses (e.g., in the lung) in a subject. In some embodiments, various dilutions of a nanoemulsion provided herein (e.g., P₄₀₇5EC) can be utilized to treat (e.g., kill and/or inhibit growth of) bacteria. In some embodiments, undiluted nanoemulsion is utilized. In some embodiments, P₄₀₇5EC is diluted (e.g., in serial, two fold dilutions) to obtain a desired concentration of one of the constituents of the nanoemulsion (e.g., CPC).

In still other embodiments of the present invention, a nanoemulsion comprises from about 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC).

In still other embodiments of the present invention, a nanoemulsion comprises from about 2 to 8 vol. % of TRITON X-100, about 8 vol. % of ethanol, about 1 vol. % of CPC, about 60 to 70 vol. % of oil (e.g., soybean, or olive oil), and about 15 to 25 vol. % of aqueous phase (e.g., DiH₂O or PBS). For example, the present invention contemplates formulations comprising about 2 vol. % of TRITON X-100, about 8 vol. % of ethanol, about 64 vol. % of soybean oil, and about 26 vol. % of DiH₂O (designated herein as X2E). In other similar embodiments, a nanoemulsion comprises about 3 vol. % of TRITON X-100, about 8 vol. % of ethanol, about 64 vol. % of soybean oil, and about 25 vol. % of DiH₂O (designated herein as X3E). In still further embodiments, the formulations comprise about 4 vol. % Triton of X-100, about 8 vol. % of ethanol, about 64 vol. % of soybean oil, and about 24 vol. % of DiH₂O (designated herein as X4E). In yet other embodiments, a nanoemulsion comprises about 5 vol. % of TRITON X-100, about 8 vol. % of ethanol, about 64 vol. % of soybean oil, and about 23 vol. % of DiH₂O (designated herein as X5E). In some embodiments, a nanoemulsion comprises about 6 vol. % of TRITON X-100, about 8 vol. % of ethanol, about 64 vol. % of soybean oil, and about 22 vol. % of DiH₂O (designated herein as X6E). In still further embodiments of the present invention, a nanoemulsion comprises about 8 vol. % of TRITON X-100, about 8 vol. % of ethanol, about 64 vol. % of soybean oil, and about 20 vol. % of DiH₂O (designated herein as X8E). In still further embodiments, a nanoemulsion comprises about 8 vol. % of TRITON X-100, about 8 vol. % of ethanol, about 64 vol. % of olive oil, and about 20 vol. % of DiH₂O (designated herein as X8E O). In yet another embodiment, a nanoemulsion comprises 8 vol. % of TRITON X-100, about 8 vol. % ethanol, about 1 vol. % CPC, about 64 vol. % of soybean oil, and about 19 vol. % of DiH₂O (designated herein as X8EC).

In alternative embodiments of the present invention, a nanoemulsion comprises from about 1 to 2 vol. % of TRITON X-100, from about 1 to 2 vol. % of TYLOXAPOL, from about 6 to 8 vol. % TBP, from about 0.5 to 1.0 vol. % of CPC, from about 60 to 70 vol. % of oil (e.g., soybean), and about 1 to 35 vol. % of aqueous phase (e.g., DiH₂O or PBS). Additionally, certain of these nanoemulsions may comprise from about 1 to 5 vol. % of trypticase soy broth, from about 0.5 to 1.5 vol. % of yeast extract, about 5 mM L-alanine/Inosine, about 10 mM ammonium chloride, and from about 20-40 vol. % of liquid baby formula. In some embodiments comprising liquid baby formula, the formula comprises a casein hydrolysate (e.g., Neutramigen, or Progestimil, and the like). In some of these embodiments, a nanoemulsion further comprises from about 0.1 to 1.0 vol. % of sodium thiosulfate, and from about 0.1 to 1.0 vol. % of sodium citrate. Other similar embodiments comprising these basic components employ phosphate buffered saline (PBS) as the aqueous phase. For example, one embodiment comprises about 2 vol. % of TRITON X-100, about 2 vol. % TYLOXAPOL, about 8 vol. % TBP, about 1 vol. % of CPC, about 64 vol. % of soybean oil, and about 23 vol. % of DiH₂O (designated herein as X2Y2EC). In still other embodiments, the inventive formulation comprises about 2 vol. % of TRITON X-100, about 2 vol. % TYLOXAPOL, about 8 vol. % TBP, about 1 vol. % of CPC, about 0.9 vol. % of sodium thiosulfate, about 0.1 vol. % of sodium citrate, about 64 vol. % of soybean oil, and about 22 vol. % of DiH₂O (designated herein as X2Y2PC STS1). In another similar embodiment, a nanoemulsion comprises about 1.7 vol. % TRITON X-100, about 1.7 vol. % TYLOXAPOL, about 6.8 vol. % TBP, about 0.85% CPC, about 29.2% NEUTRAMIGEN, about 54.4 vol. % of soybean oil, and about 4.9 vol. % of DiH₂O (designated herein as 85% X2Y2PC/baby). In yet another embodiment of the present invention, a nanoemulsion comprises about 1.8 vol. % of TRITON X-100, about 1.8 vol. % of TYLOXAPOL, about 7.2 vol. % of TBP, about 0.9 vol. % of CPC, about 5 mM L-alanine/Inosine, about 10 mM ammonium chloride, about 57.6 vol. % of soybean oil, and the remainder vol. % of 0.1×PBS (designated herein as 90% X2Y2 PC/GE). In still another embodiment, a nanoemulsion comprises about 1.8 vol. % of TRITON X-100, about 1.8 vol. % of TYLOXAPOL, about 7.2 vol. % TBP, about 0.9 vol. % of CPC, and about 3 vol. % trypticase soy broth, about 57.6 vol. % of soybean oil, and about 27.7 vol. % of DiH₂O (designated herein as 90% X2Y2PC/TSB). In another embodiment of the present invention, a nanoemulsion comprises about 1.8 vol. % TRITON X-100, about 1.8 vol. % TYLOXAPOL, about 7.2 vol. % TBP, about 0.9 vol. % CPC, about 1 vol. % yeast extract, about 57.6 vol. % of soybean oil, and about 29.7 vol. % of DiH₂O (designated herein as 90% X2Y2PC/YE).

In some embodiments of the present invention, a nanoemulsion comprises about 3 vol. % of TYLOXAPOL, about 8 vol. % of TBP, and about 1 vol. % of CPC, about 60 to 70 vol. % of oil (e.g., soybean or olive oil), and about 15 to 30 vol. % of aqueous phase (e.g., DiH₂O or PBS). In a particular embodiment of the present invention, a nanoemulsion comprises about 3 vol. % of TYLOXAPOL, about 8 vol. % of TBP, and about 1 vol. % of CPC, about 64 vol. % of soybean, and about 24 vol. % of DiH₂O (designated herein as Y3PC).

In some embodiments of the present invention, a nanoemulsion comprises from about 4 to 8 vol. % of TRITON X-100, from about 5 to 8 vol. % of TBP, about 30 to 70 vol. % of oil (e.g., soybean or olive oil), and about 0 to 30 vol. % of aqueous phase (e.g., DiH₂O or PBS). Additionally, certain of these embodiments further comprise about 1 vol. % of CPC, about 1 vol. % of benzalkonium chloride, about 1 vol. % cetylyridinium bromide, about 1 vol. % cetyldimethyletylammonium bromide, 500 μM EDTA, about 10 mM ammonium chloride, about 5 mM Inosine, and about 5 mM L-alanine. For example, in a certain preferred embodiment, a nanoemulsion comprises about 8 vol. % of TRITON X-100, about 8 vol. % of TBP, about 64 vol. % of soybean oil, and about 20 vol. % of DiH₂O (designated herein as X8P). In another embodiment of the present invention, a nanoemulsion comprises about 8 vol. % of TRITON X-100, about 8 vol. % of TBP, about 1% of CPC, about 64 vol. % of soybean oil, and about 19 vol. % of DiH₂O (designated herein as X8PC). In still another embodiment, a nanoemulsion comprises about 8 vol. % TRITON X-100, about 8 vol. % of TBP, about 1 vol. % of CPC, about 50 vol. % of soybean oil, and about 33 vol. % of DiH₂O (designated herein as ATB-X1001). In yet another embodiment, the formulations comprise about 8 vol. % of TRITON X-100, about 8 vol. % of TBP, about 2 vol. % of CPC, about 50 vol. % of soybean oil, and about 32 vol. % of DiH₂O (designated herein as ATB-X002). In some embodiments, a nanoemulsion comprises about 4 vol. % TRITON X-100, about 4 vol. % of TBP, about 0.5 vol. % of CPC, about 32 vol. % of soybean oil, and about 59.5 vol. % of DiH₂O (designated herein as 50% X8PC). In some embodiments, a nanoemulsion comprises about 8 vol. % of TRITON X-100, about 8 vol. % of TBP, about 0.5 vol. % CPC, about 64 vol. % of soybean oil, and about 19.5 vol. % of DiH₂O (designated herein as X8PC_{1/2}). In some embodiments of the present invention, a nanoemulsion comprises about 8 vol. % of TRITON X-100, about 8 vol. % of TBP, about 2 vol. % of CPC, about 64 vol. % of soybean oil, and about 18 vol. % of DiH₂O (designated herein as X8PC2). In other embodiments, a nanoemulsion comprises about 8 vol. % of TRITON X-100, about 8% of TBP, about 1% of benzalkonium chloride, about 50 vol. % of soybean oil, and about 33 vol. % of DiH₂O (designated herein as X8P BC). In an alternative embodiment of the present invention, a nanoemulsion comprises about 8 vol. % of TRITON X-100, about 8 vol. % of TBP, about 1 vol. % of cetylyridinium bromide, about 50 vol. % of soybean oil, and about 33 vol. % of DiH₂O (designated herein as X8P CPB). In another exemplary embodiment of the present invention, a nanoemulsion comprises about 8 vol. % of TRITON X-100, about 8 vol. % of TBP, about 1 vol. % of cetyldimethyletylammonium bromide, about 50 vol. % of soybean oil, and about 33 vol. % of DiH₂O (designated herein as X8P CTAB). In still further embodiments, a nanoemulsion comprises about 8 vol. % of TRITON X-100, about 8 vol. % of TBP, about 1 vol. % of CPC, about 500 μM EDTA, about 64 vol. % of soybean oil, and about 15.8 vol. % DiH₂O (designated herein as X8PC EDTA). In some embodiments, a nanoemulsion comprises 8 vol. % of TRITON X-100, about 8 vol. % of TBP, about 1 vol. % of CPC, about 10 mM ammonium chloride, about 5 mM Inosine, about 5 mM L-alanine, about 64 vol. % of soybean oil, and about 19 vol. % of DiH₂O or PBS (designated herein as X8PC GE₁ₓ). In another embodiment of the present invention, a nanoemulsion comprises about 5 vol. % of TRITON X-100, about 5% of TBP, about 1 vol. % of CPC, about 40 vol. % of soybean oil, and about 49 vol. % of DiH₂O (designated herein as X5P₅C).

In some embodiments of the present invention, a nanoemulsion comprises about 2 vol. % TRITON X-100, about 6 vol. % TYLOXAPOL, about 8 vol. % ethanol, about 64 vol. % of soybean oil, and about 20 vol. % of DiH₂O (designated herein as X2Y6E).

In an additional embodiment of the present invention, a nanoemulsion comprises about 8 vol. % of TRITON X-100, and about 8 vol. % of glycerol, about 60 to 70 vol. % of oil (e.g., soybean or olive oil), and about 15 to 25 vol. % of aqueous phase (e.g., DiH₂O or PBS). Certain nanoemulsion compositions (e.g., used to generate an immune response (e.g., for use as a vaccine) comprise about 1 vol. % L-ascorbic acid. For example, one particular embodiment comprises about 8 vol. % of TRITON X-100, about 8 vol. % of glycerol, about 64 vol. % of soybean oil, and about 20 vol. % of DiH₂O (designated herein as X8G). In still another embodiment, a nanoemulsion comprises about 8 vol. % of TRITON X-100, about 8 vol. % of glycerol, about 1 vol. % of L-ascorbic acid, about 64 vol. % of soybean oil, and about 19 vol. % of DiH₂O (designated herein as X8GV_{c}).

In still further embodiments, a nanoemulsion comprises about 8 vol. % of TRITON X-100, from about 0.5 to 0.8 vol. % of TWEEN 60, from about 0.5 to 2.0 vol. % of CPC, about 8 vol. % of TBP, about 60 to 70 vol. % of oil (e.g., soybean or olive oil), and about 15 to 25 vol. % of aqueous phase (e.g., DiH₂O or PBS). For example, in one particular embodiment a nanoemulsion comprises about 8 vol. % of TRITON X-100, about 0.70 vol. % of TWEEN 60, about 1 vol. % of CPC, about 8 vol. % of TBP, about 64 vol. % of soybean oil, and about 18.3 vol. % of DiH₂O (designated herein as X8W60PC₁). In some embodiments, a nanoemulsion comprises about 8 vol. % of TRITON X-100, about 0.71 vol. % of TWEEN 60, about 1 vol. % of CPC, about 8 vol. % of TBP, about 64 vol. % of soybean oil, and about 18.29 vol. % of DiH₂O (designated herein as W60_{0.7}X8PC). In yet other embodiments, a nanoemulsion comprises from about 8 vol. % of TRITON X-100, about 0.7 vol. % of TWEEN 60, about 0.5 vol. % of CPC, about 8 vol. % of TBP, about 64 to 70 vol. % of soybean oil, and about 18.8 vol. % of DiH₂O (designated herein as X8W60PC₂). In still other embodiments, a nanoemulsion comprises about 8 vol. % of TRITON X-100, about 0.71 vol. % of TWEEN 60, about 2 vol. % of CPC, about 8 vol. % of TBP, about 64 vol. % of soybean oil, and about 17.3 vol. % of DiH₂O. In another embodiment of the present invention, a nanoemulsion comprises about 0.71 vol. % of TWEEN 60, about 1 vol. % of CPC, about 8 vol. % of TBP, about 64 vol. % of soybean oil, and about 25.29 vol. % of DiH₂O (designated herein as W60_{0.7}PC).

In another embodiment of the present invention, a nanoemulsion comprises about 2 vol. % of dioctyl sulfosuccinate, either about 8 vol. % of glycerol, or about 8 vol. % TBP, in addition to, about 60 to 70 vol. % of oil (e.g., soybean or olive oil), and about 20 to 30 vol. % of aqueous phase (e.g., DiH₂O or PBS). For example, in some embodiments, a nanoemulsion comprises about 2 vol. % of dioctyl sulfosuccinate, about 8 vol. % of glycerol, about 64 vol. % of soybean oil, and about 26 vol. % of D1H₂O (designated herein as D2G). In another related embodiment, a nanoemulsion comprises about 2 vol. % of dioctyl sulfosuccinate, and about 8 vol. % of TBP, about 64 vol. % of soybean oil, and about 26 vol. % of D1H₂O (designated herein as D2P).

In still other embodiments of the present invention, a nanoemulsion comprises about 8 to 10 vol. % of glycerol, and about 1 to 10 vol. % of CPC, about 50 to 70 vol. % of oil (e.g., soybean or olive oil), and about 15 to 30 vol. % of aqueous phase (e.g., DiH₂O or PBS). Additionally, in certain of these embodiments, a nanoemulsion further comprises about 1 vol. % of L-ascorbic acid. For example, in some embodiments, a nanoemulsion comprises about 8 vol. % of glycerol, about 1 vol. % of CPC, about 64 vol. % of soybean oil, and about 27 vol. % of DiH₂O (designated herein as GC). In some embodiments, a nanoemulsion comprises about 10 vol. % of glycerol, about 10 vol. % of CPC, about 60 vol. % of soybean oil, and about 20 vol. % of DiH₂O (designated herein as GC10). In still another embodiment of the present invention, a nanoemulsion comprises about 10 vol. % of glycerol, about 1 vol. % of CPC, about 1 vol. % of L-ascorbic acid, about 64 vol. % of soybean or oil, and about 24 vol. % of DiH₂O (designated herein as GCV_{c}).

In some embodiments of the present invention, a nanoemulsion comprises about 8 to 10 vol. % of glycerol, about 8 to 10 vol. % of SDS, about 50 to 70 vol. % of oil (e.g., soybean or olive oil), and about 15 to 30 vol. % of aqueous phase (e.g., DiH₂O or PBS). Additionally, in certain of these embodiments, a nanoemulsion further comprise about 1 vol. % of lecithin, and about 1 vol. % of p-Hydroxybenzoic acid methyl ester. Exemplary embodiments of such formulations comprise about 8 vol. % SDS, 8 vol. % of glycerol, about 64 vol. % of soybean oil, and about 20 vol. % of DiH₂O (designated herein as S8G). A related formulation comprises about 8 vol. % of glycerol, about 8 vol. % of SDS, about 1 vol. % of lecithin, about 1 vol. % of p-Hydroxybenzoic acid methyl ester, about 64 vol. % of soybean oil, and about 18 vol. % of DiH₂O (designated herein as S8GL1B1).

In yet another embodiment of the present invention, a nanoemulsion comprises about 4 vol. % of TWEEN 80, about 4 vol. % of TYLOXAPOL, about 1 vol. % of CPC, about 8 vol. % of ethanol, about 64 vol. % of soybean oil, and about 19 vol. % of DiH₂O (designated herein as W₈₀₄Y4EC).

In some embodiments of the present invention, a nanoemulsion comprises about 0.01 vol. % of CPC, about 0.08 vol. % of TYLOXAPOL, about 10 vol. % of ethanol, about 70 vol. % of soybean oil, and about 19.91 vol. % of DiH₂O (designated herein as Y.08EC.01).

In yet another embodiment of the present invention, a nanoemulsion comprises about 8 vol. % of sodium lauryl sulfate, and about 8 vol. % of glycerol, about 64 vol. % of soybean oil, and about 20 vol. % of DiH₂O (designated herein as SLS8G).

The specific formulations described above are simply examples to illustrate the variety of nanoemulsion adjuvants that find use in the present invention. The present invention contemplates that many variations of the above formulations, as well as additional nanoemulsions, find use in the methods of the present invention. Candidate emulsions can be easily tested to determine if they are suitable. First, the desired ingredients are prepared using the methods described herein, to determine if an emulsion can be formed. If an emulsion cannot be formed, the candidate is rejected. For example, a candidate composition made of 4.5% sodium thiosulfate, 0.5% sodium citrate, 10% n-butanol, 64% soybean oil, and 21% DiH₂O does not form an emulsion.

Second, the candidate emulsion should form a stable emulsion. An emulsion is stable if it remains in emulsion form for a sufficient period to allow its intended use (e.g., to generate an immune response in a subject). For example, for emulsions that are to be stored, shipped, etc., it may be desired that the composition remain in emulsion form for months to years. Typical emulsions that are relatively unstable, will lose their form within a day. For example, a candidate composition made of 8% 1-butanol, 5% TWEEN 10, 1% CPC, 64% soybean oil, and 22% DiH₂O does not form a stable emulsion. Nanoemulsions that have been shown to be stable include, but are not limited to, 8 vol. % of TRITON X-100, about 8 vol. % of TBP, about 64 vol. % of soybean oil, and about 20 vol. % of DiH₂O (designated herein as X8P); 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC); 0.08% Triton X-100, 0.08% Glycerol, 0.01% Cetylpyridinium Chloride, 99% Butter, and 0.83% diH₂O (designated herein as 1% X8GC Butter); 0.8% Triton X-100, 0.8% Glycerol, 0.1% Cetylpyridinium Chloride, 6.4% Soybean Oil, 1.9% diH₂O, and 90% Butter (designated herein as 10% X8GC Butter); 2% W₂₀5EC, 1% Natrosol 250L NF, and 97% diH₂O (designated herein as 2% W₂₀5EC L GEL); 1% Cetylpyridinium Chloride, 5% TWEEN 20, 8% Ethanol, 64% 70 Viscosity Mineral Oil, and 22% diH₂O (designated herein as W₂₀5EC 70 Mineral Oil); 1% Cetylpyridinium Chloride, 5% TWEEN 20, 8% Ethanol, 64% 350 Viscosity Mineral Oil, and 22% diH₂O (designated herein as W₂₀5EC 350 Mineral Oil). In some embodiments, nanoemulsions of the present invention are stable for over a week, over a month, or over a year.

Third, the candidate emulsion should have efficacy for its intended use. For example, a nanoemulsion should inactivate (e.g., kill or inhibit growth of) a pathogen to a desired level (e.g., 1 log, 2 log, 3 log, 4 log, . . . reduction). Using the methods described herein, one is capable of determining the suitability of a particular candidate emulsion against the desired pathogen. Generally, this involves exposing the pathogen to the emulsion for one or more time periods in a side-by-side experiment with the appropriate control samples (e.g., a negative control such as water) and determining if, and to what degree, the emulsion inactivates (e.g., kills and/or neutralizes) the microorganism. For example, a candidate composition made of 1% ammonium chloride, 5% TWEEN 20, 8% ethanol, 64% soybean oil, and 22% DiH₂O was shown not to be an effective emulsion. The following candidate emulsions were shown to be effective using the methods described herein: 5% TWEEN 20, 5% Cetylpyridinium Chloride, 10% Glycerol, 60% Soybean Oil, and 20% diH₂O (designated herein as W₂₀5GC5); 1% Cetylpyridinium Chloride, 5% TWEEN 20, 10% Glycerol, 64% Soybean Oil, and 20% diH₂O (designated herein as W₂₀5GC); 1% Cetylpyridinium Chloride, 5% TWEEN 20, 8% Ethanol, 64% Olive Oil, and 22% diH₂O (designated herein as W₂₀5EC Olive Oil); 1% Cetylpyridinium Chloride, 5% TWEEN 20, 8% Ethanol, 64% Flaxseed Oil, and 22% diH₂O (designated herein as W₂₀5EC Flaxseed Oil); 1% Cetylpyridinium Chloride, 5% TWEEN 20, 8% Ethanol, 64% Corn Oil, and 22% diH₂O (designated herein as W₂₀5EC Corn Oil); 1% Cetylpyridinium Chloride, 5% TWEEN 20, 8% Ethanol, 64% Coconut Oil, and 22% diH₂O (designated herein as W₂₀5EC Coconut Oil); 1% Cetylpyridinium Chloride, 5% TWEEN 20, 8% Ethanol, 64% Cottonseed Oil, and 22% diH₂O (designated herein as W₂₀5EC Cottonseed Oil); 8% Dextrose, 5% TWEEN 10, 1% Cetylpyridinium Chloride, 64% Soybean Oil, and 22% diH₂O (designated herein as W₂₀5C Dextrose); 8% PEG 200, 5% TWEEN 10, 1% Cetylpyridinium Chloride, 64% Soybean Oil, and 22% diH₂O (designated herein as W₂₀5C PEG 200); 8% Methanol, 5% TWEEN 10, 1% Cetylpyridinium Chloride, 64% Soybean Oil, and 22% diH₂O (designated herein as W₂₀5C Methanol); 8% PEG 1000, 5% TWEEN 10, 1% Cetylpyridinium Chloride, 64% Soybean Oil, and 22% diH₂O (designated herein as W₂₀5C PEG 1000); 2% W₂₀5EC, 2% Natrosol 250H NF, and 96% diH₂O (designated herein as 2% W₂₀5EC Natrosol 2, also called 2% W₂₀5EC GEL); 2% W₂₀5EC, 1% Natrosol 250H NF, and 97% diH₂O (designated herein as 2% W₂₀5EC Natrosol 1); 2% W₂₀5EC, 3% Natrosol 250H NF, and 95% diH₂O (designated herein as 2% W₂₀5EC Natrosol 3); 2% W₂₀5EC, 0.5% Natrosol 250H NF, and 97.5% diH₂O (designated herein as 2% W₂₀5EC Natrosol 0.5); 2% W₂₀5EC, 2% Methocel A, and 96% diH₂O (designated herein as 2% W₂₀5EC Methocel A); 2% W₂₀5EC, 2% Methocel K, and 96% diH₂O (designated herein as 2% W₂₀5EC Methocel K); 2% Natrosol, 0.1% X8PC, 0.1×PBS, 5 mM L-alanine, 5 mM Inosine, 10 mM Ammonium Chloride, and diH₂O (designated herein as 0.1% X8PC/GE+2% Natrosol); 2% Natrosol, 0.8% Triton X-100, 0.8% Tributyl Phosphate, 6.4% Soybean Oil, 0.1% Cetylpyridinium Chloride, 0.1×PBS, 5 mM L-alanine, 5 mM Inosine, 10 mM Ammonium Chloride, and diH₂O (designated herein as 10% X8PC/GE+2% Natrosol); 1% Cetylpyridinium Chloride, 5% TWEEN 20, 8% Ethanol, 64% Lard, and 22% diH₂O (designated herein as W₂₀5EC Lard); 1% Cetylpyridinium Chloride, 5% TWEEN 20, 8% Ethanol, 64% Mineral Oil, and 22% diH₂O (designated herein as W₂₀5EC Mineral Oil); 0.1% Cetylpyridinium Chloride, 2% Nerolidol, 5% TWEEN 20, 10% Ethanol, 64% Soybean Oil, and 18.9% diH₂O (designated herein as W₂₀5EC_{0.1}N); 0.1% Cetylpyridinium Chloride, 2% Farnesol, 5% TWEEN 20, 10% Ethanol, 64% Soybean Oil, and 18.9% diH₂O (designated herein as W₂₀5EC_{0.1}F); 0.1% Cetylpyridinium Chloride, 5% TWEEN 20, 10% Ethanol, 64% Soybean Oil, and 20.9% diH₂O (designated herein as W₂₀5EC_{0.1}); 10% Cetylpyridinium Chloride, 8% Tributyl Phosphate, 8% Triton X-100, 54% Soybean Oil, and 20% diH₂O (designated herein as X8PC₁₀); 5% Cetylpyridinium Chloride, 8% Triton X-100, 8% Tributyl Phosphate, 59% Soybean Oil, and 20% diH₂O (designated herein as X8PC₅); 0.02% Cetylpyridinium Chloride, 0.1% TWEEN 20, 10% Ethanol, 70% Soybean Oil, and 19.88% diH₂O (designated herein as W₂₀0.1EC_{0.02}); 1% Cetylpyridinium Chloride, 5% TWEEN 20, 8% Glycerol, 64% Mobil 1, and 22% diH₂O (designated herein as W₂₀5GC Mobil 1); 7.2% Triton X-100, 7.2% Tributyl Phosphate, 0.9% Cetylpyridinium Chloride, 57.6% Soybean Oil, 0.1×PBS, 5 mM L-alanine, 5 mM Inosine, 10 mM Ammonium Chloride, and 25.87% diH₂O (designated herein as 90% X8PC/GE); 7.2% Triton X-100, 7.2% Tributyl Phosphate, 0.9% Cetylpyridinium Chloride, 57.6% Soybean Oil, 1% EDTA, 5 mM L-alanine, 5 mM Inosine, 10 mM Ammonium Chloride, 0.1×PBS, and diH₂O (designated herein as 90% X8PC/GE EDTA); and 7.2% Triton X-100, 7.2% Tributyl Phosphate, 0.9% Cetylpyridinium Chloride, 57.6% Soybean Oil, 1% Sodium Thiosulfate, 5 mM L-alanine, 5 mM Inosine, 10 mM Ammonium Chloride, 0.1×PBS, and diH₂O (designated herein as 90% X8PC/GE STS).

In preferred embodiments of the present invention, the nanoemulsions are non-toxic (e.g., to humans, plants, or animals), non-irritant (e.g., to humans, plants, or animals), and non-corrosive (e.g., to humans, plants, or animals or the environment), while retaining stability when mixed with other agents (e.g., a composition comprising an immunogen (e.g., bacteria, fungi, viruses, and spores). While a number of the above described nanoemulsions meet these qualifications, the following description provides a number of preferred non-toxic, non-irritant, non-corrosive, anti-microbial nanoemulsions of the present invention (hereinafter in this section referred to as “non-toxic nanoemulsions”).

In some embodiments the non-toxic nanoemulsions comprise surfactant lipid preparations (SLPs) for use as broad-spectrum antimicrobial agents that are effective against bacteria and their spores, enveloped viruses, and fungi. In preferred embodiments, these SLPs comprise a mixture of oils, detergents, solvents, and cationic halogen-containing compounds in addition to several ions that enhance their biocidal activities. These SLPs are characterized as stable, non-irritant, and non-toxic compounds compared to commercially available bactericidal and sporicidal agents, which are highly irritant and/or toxic.

Ingredients for use in the non-toxic nanoemulsions include, but are not limited to: detergents (e.g., TRITON X-100 (5-15%) or other members of the TRITON family, TWEEN 60 (0.5-2%) or other members of the TWEEN family, or TYLOXAPOL (1-10%)); solvents (e.g., tributyl phosphate (5-15%)); alcohols (e.g., ethanol (5-15%) or glycerol (5-15%)); oils (e.g., soybean oil (40-70%)); cationic halogen-containing compounds (e.g., cetylpyridinium chloride (0.5-2%), cetylpyridinium bromide (0.5-2%)), or cetyldimethylethyl ammonium bromide (0.5-2%)); quaternary ammonium compounds (e.g., benzalkonium chloride (0.5-2%), N-alkyldimethylbenzyl ammonium chloride (0.5-2%)); ions (calcium chloride (1 mM-40 mM), ammonium chloride (1 mM-20 mM), sodium chloride (5 mM-200 mM), sodium phosphate (1 mM-20 mM)); nucleosides (e.g., inosine (50 μM-20 mM)); and amino acids (e.g., L-alanine (50 μM-20 mM)). Emulsions are prepared, for example, by mixing in a high shear mixer for 3-10 minutes. The emulsions may or may not be heated before mixing at 82° C. for 1 hour.

Quaternary ammonium compounds for use in the present include, but are not limited to, N-alkyldimethyl benzyl ammonium saccharinate; 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C₈₋₁₀)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride.

1. Aqueous Phase

In some embodiments, the emulsion comprises an aqueous phase. In certain preferred embodiments, the emulsion comprises about 5 to 50, preferably 10 to 40, more preferably 15 to 30, vol. % aqueous phase, based on the total volume of the emulsion (although other concentrations are also contemplated). In preferred embodiments, the aqueous phase comprises water at a pH of about 4 to 10, preferably about 6 to 8. The water is preferably deionized (hereinafter “DiH₂O”). In some embodiments, the aqueous phase comprises phosphate buffered saline (PBS). In some preferred embodiments, the aqueous phase is sterile and pyrogen free.

2. Oil Phase

In some embodiments, the emulsion comprises an oil phase. In certain preferred embodiments, the oil phase (e.g., carrier oil) of the emulsion of the present invention comprises 30-90, preferably 60-80, and more preferably 60-70, vol. % of oil, based on the total volume of the emulsion (although higher and lower concentrations also find use in emulsions described herein).

The oil in the nanoemulsion adjuvant of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

In some embodiments, the oil phase comprises 3-15, and preferably 5-10 vol. % of an organic solvent, based on the total volume of the emulsion. While the present invention is not limited to any particular mechanism, it is contemplated that the organic phosphate-based solvents employed in the emulsions serve to remove or disrupt the lipids in the membranes of the pathogens. Thus, any solvent that removes the sterols or phospholipids in the microbial membranes finds use in the methods of the present invention. Suitable organic solvents include, but are not limited to, organic phosphate based solvents or alcohols. In some preferred embodiments, non-toxic alcohols (e.g., ethanol) are used as a solvent. The oil phase, and any additional compounds provided in the oil phase, are preferably sterile and pyrogen free.

3. Surfactants and Detergents

In some embodiments, the emulsions further comprises a surfactant or detergent. In some preferred embodiments, the emulsion comprises from about 3 to 15%, and preferably about 10% of one or more surfactants or detergents (although other concentrations are also contemplated). While the present invention is not limited to any particular mechanism, it is contemplated that surfactants, when present in the emulsions, help to stabilize the emulsions. Both non-ionic (non-anionic) and ionic surfactants are contemplated. Additionally, surfactants from the BRIJ family of surfactants find use in the compositions of the present invention. The surfactant can be provided in either the aqueous or the oil phase. Surfactants suitable for use with the emulsions include a variety of anionic and nonionic surfactants, as well as other emulsifying compounds that are capable of promoting the formation of oil-in-water emulsions. In general, emulsifying compounds are relatively hydrophilic, and blends of emulsifying compounds can be used to achieve the necessary qualities. In some formulations, nonionic surfactants have advantages over ionic emulsifiers in that they are substantially more compatible with a broad pH range and often form more stable emulsions than do ionic (e.g., soap-type) emulsifiers.

The surfactant in the nanoemulsion adjuvant of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference. Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23. In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis(imidazoyl carbonyl)), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

The present invention is not limited to the surfactants disclosed herein. Additional surfactants and detergents useful in the compositions of the present invention may be ascertained from reference works (e.g., including, but not limited to, McCutheon's Volume 1: Emulsions and Detergents—North American Edition, 2000) and commercial sources.

4. Cationic Halogens Containing Compounds

In some embodiments, the emulsions further comprise a cationic halogen containing compound. In some preferred embodiments, the emulsion comprises from about 0.5 to 1.0 wt. % or more of a cationic halogen containing compound, based on the total weight of the emulsion (although other concentrations are also contemplated). In preferred embodiments, the cationic halogen-containing compound is preferably premixed with the oil phase; however, it should be understood that the cationic halogen-containing compound may be provided in combination with the emulsion composition in a distinct formulation. Suitable halogen containing compounds may be selected from compounds comprising chloride, fluoride, bromide and iodide ions. In preferred embodiments, suitable cationic halogen containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), and cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen-containing compound is CPC, although the compositions of the present invention are not limited to formulation with any particular cationic containing compound.

5. Germination Enhancers

In other embodiments of the present invention, the nanoemulsions further comprise a germination enhancer. In some preferred embodiments, the emulsions comprise from about 1 mM to 15 mM, and more preferably from about 5 mM to 10 mM of one or more germination enhancing compounds (although other concentrations are also contemplated). In preferred embodiments, the germination enhancing compound is provided in the aqueous phase prior to formation of the emulsion. The present invention contemplates that when germination enhancers are added to the nanoemulsion compositions, the sporicidal properties of the nanoemulsions are enhanced. The present invention further contemplates that such germination enhancers initiate sporicidal activity near neutral pH (between pH 6-8, and preferably 7). Such neutral pH emulsions can be obtained, for example, by diluting with phosphate buffer saline (PBS) or by preparations of neutral emulsions. The sporicidal activity of the nanoemulsion preferentially occurs when the spores initiate germination.

In specific embodiments, it has been demonstrated that the emulsions utilized in the vaccines of the present invention have sporicidal activity. While the present invention is not limited to any particular mechanism and an understanding of the mechanism is not required to practice the present invention, it is believed that the fusigenic component of the emulsions acts to initiate germination and before reversion to the vegetative form is complete the lysogenic component of the emulsion acts to lyse the newly germinating spore. These components of the emulsion thus act in concert to leave the spore susceptible to disruption by the emulsions. The addition of germination enhancer further facilitates the anti-sporicidal activity of the emulsions, for example, by speeding up the rate at which the sporicidal activity occurs.

Germination of bacterial endospores and fungal spores is associated with increased metabolism and decreased resistance to heat and chemical reactants. For germination to occur, the spore must sense that the environment is adequate to support vegetation and reproduction. The amino acid L-alanine stimulates bacterial spore germination (See e.g., Hills, J. Gen. Micro. 4:38 (1950); and Halvorson and Church, Bacteriol Rev. 21:112 (1957)). L-alanine and L-proline have also been reported to initiate fungal spore germination (Yanagita, Arch Mikrobiol 26:329 (1957)). Simple α-amino acids, such as glycine and L-alanine, occupy a central position in metabolism. Transamination or deamination of α-amino acids yields the glycogenic or ketogenic carbohydrates and the nitrogen needed for metabolism and growth. For example, transamination or deamination of L-alanine yields pyruvate, which is the end product of glycolytic metabolism (Embden-Meyerhof Pathway). Oxidation of pyruvate by pyruvate dehydrogenase complex yields acetyl-CoA, NADH, H⁺, and CO₂. Acetyl-CoA is the initiator substrate for the tricarboxylic acid cycle (Kreb's Cycle), which in turns feeds the mitochondrial electron transport chain. Acetyl-CoA is also the ultimate carbon source for fatty acid synthesis as well as for sterol synthesis. Simple α-amino acids can provide the nitrogen, CO₂, glycogenic and/or ketogenic equivalents required for germination and the metabolic activity that follows.

In certain embodiments, suitable germination enhancing agents of the invention include, but are not limited to, α-amino acids comprising glycine and the L-enantiomers of alanine, valine, leucine, isoleucine, serine, threonine, lysine, phenylalanine, tyrosine, and the alkyl esters thereof. Additional information on the effects of amino acids on germination may be found in U.S. Pat. No. 5,510,104; herein incorporated by reference in its entirety. In some embodiments, a mixture of glucose, fructose, asparagine, sodium chloride (NaCl), ammonium chloride (NH₄Cl), calcium chloride (CaCl₂) and potassium chloride (KCl) also may be used. In particularly preferred embodiments of the present invention, the formulation comprises the germination enhancers L-alanine, CaCl₂, Inosine and NH₄Cl. In some embodiments, the compositions further comprise one or more common forms of growth media (e.g., trypticase soy broth, and the like) that additionally may or may not itself comprise germination enhancers and buffers.

The above compounds are merely exemplary germination enhancers and it is understood that other known germination enhancers will find use in the nanoemulsions utilized in some embodiments of the present invention. A candidate germination enhancer should meet two criteria for inclusion in the compositions of the present invention: it should be capable of being associated with the emulsions disclosed herein and it should increase the rate of germination of a target spore when incorporated in the emulsions disclosed herein. One skilled in the art can determine whether a particular agent has the desired function of acting as an germination enhancer by applying such an agent in combination with the nanoemulsions disclosed herein to a target and comparing the inactivation of the target when contacted by the admixture with inactivation of like targets by the composition of the present invention without the agent. Any agent that increases germination, and thereby decreases or inhibits the growth of the organisms, is considered a suitable enhancer for use in the nanoemulsion compositions disclosed herein.

In still other embodiments, addition of a germination enhancer (or growth medium) to a neutral emulsion composition produces a composition that is useful in inactivating bacterial spores in addition to enveloped viruses, Gram negative bacteria, and Gram positive bacteria for use in the vaccine compositions of the present invention.

6. Interaction Enhancers

In still other embodiments, nanoemulsions comprise one or more compounds capable of increasing the interaction of the compositions (i.e., “interaction enhancer” (e.g., with target pathogens (e.g., the cell wall of Gram negative bacteria such as Vibrio, Salmonella, Shigella and Pseudomonas)). In preferred embodiments, the interaction enhancer is preferably premixed with the oil phase; however, in other embodiments the interaction enhancer is provided in combination with the compositions after emulsification. In certain preferred embodiments, the interaction enhancer is a chelating agent (e.g., ethylenediaminetetraacetic acid (EDTA) or ethylenebis(oxyethylenenitrilo)tetraacetic acid (EGTA) in a buffer (e.g., tris buffer)). It is understood that chelating agents are merely exemplary interaction enhancing compounds. Indeed, other agents that increase the interaction of the nanoemulsions used in some embodiments of the present invention (e.g., with microbial agents, pathogens, vaccines, etc.) are contemplated. In particularly preferred embodiments, the interaction enhancer is at a concentration of about 50 to about 250 μM. One skilled in the art will be able to determine whether a particular agent has the desired function of acting as an interaction enhancer by applying such an agent in combination with the compositions of the present invention to a target and comparing the inactivation of the target when contacted by the admixture with inactivation of like targets by the composition of the present invention without the agent. Any agent that increases the interaction of an emulsion with bacteria and thereby decreases or inhibits the growth of the bacteria, in comparison to that parameter in its absence, is considered an interaction enhancer.

In some embodiments, the addition of an interaction enhancer to nanoemulsion produces a composition that is useful in inactivating enveloped viruses, some Gram positive bacteria and some Gram negative bacteria for use in a vaccine composition.

7. Quaternary Ammonium Compounds

In some embodiments, nanoemulsions of the present invention include a quaternary ammonium containing compound. Exemplary quaternary ammonium compounds include, but are not limited to, Alkyl dimethyl benzyl ammonium chloride, didecyl dimethyl ammonium chloride, Alkyl dimethyl benzyl and dialkyl dimethyl ammonium chloride, N,N-Dimethyl-2-hydroxypropylammonium chloride polymer, Didecyl dimethyl ammonium chloride, n-Alkyl dimethyl benzyl ammonium chloride, n-Alkyl dimethyl ethylbenzyl ammonium chloride, Dialkyl dimethyl ammonium chloride, n-Alkyl dimethyl benzyl ammonium chloride, n-Tetradecyl dimethyl benzyl ammonium chloride monohydrate, n-Alkyl dimethyl benzyl ammonium chloride, Dialkyl dimethyl ammonium chloride, Hexahydro-1,3,5-tris(2-hydroxyethyl)-s-triazine, Myristalkonium chloride (and) Quat RNIUM 14, Alkyl bis(2-hydroxyethyl) benzyl ammonium chloride, Alkyl demethyl benzyl ammonium chloride, Alkyl dimethyl 3,4-dichlorobenzyl ammonium chloride, Alkyl dimethyl benzyl ammonium chloride, Alkyl dimethyl benzyl dimethylbenzyl ammonium, Alkyl dimethyl dimethybenzyl ammonium chloride, Alkyl dimethyl ethyl ammonium bromide, Alkyl dimethyl ethyl ammonium bromide, Alkyl dimethyl ethylbenzyl ammonium chloride, Alkyl dimethyl isopropylbenzyl ammonium chloride, Alkyl trimethyl ammonium chloride, Alkyl 1 or 3 benzyl-1-(2-hydroxethyl)-2-imidazolinium chloride, Dialkyl methyl benzyl ammonium chloride, Dialkyl dimethyl ammonium chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysilyl quats, and Trimethyl dodecylbenzyl ammonium chloride.

8. Other Components

In some embodiments, a nanoemulsion adjuvant composition comprises one or more additional components that provide a desired property or functionality to the nanoemulsions. These components may be incorporated into the aqueous phase or the oil phase of the nanoemulsions and/or may be added prior to or following emulsification. For example, in some embodiments, the nanoemulsions further comprise phenols (e.g., triclosan, phenyl phenol), acidifying agents (e.g., citric acid (e.g., 1.5-6%), acetic acid, lemon juice), alkylating agents (e.g., sodium hydroxide (e.g., 0.3%)), buffers (e.g., citrate buffer, acetate buffer, and other buffers useful to maintain a specific pH), and halogens (e.g., polyvinylpyrrolidone, sodium hypochlorite, hydrogen peroxide).

Exemplary techniques for making a nanoemulsion are described below. Additionally, a number of specific, although exemplary, formulation recipes are also set forth herein.

In some embodiments, a nanoemulsion adjuvant is administered to a subject before, concurrent with or after administration of a composition comprising an immunogen (e.g., a pathogen and/or pathogen component (e.g., purified, isolated and/or recombinant pathogen peptide and/or protein)). The invention is not limited to the use of any one specific type of composition comprising an immunogen. Indeed, a variety of compositions comprising an immunogen (e.g., utilized for generating an immune response (e.g., for use as a vaccine)) may be utilized with a nanoemulsion adjuvant of the invention. In some embodiments, the composition comprising an immunogen comprises pathogens (e.g., killed pathogens), pathogen components or isolated, purified and/or recombinant parts thereof. Accordingly, in some embodiments, the composition comprising an immunogen comprises a bacterial pathogen or pathogen component including, but not limited to, Bacillus cereus, Bacillus circulans and Bacillus megaterium, Bacillus anthracis, bacteria of the genus Brucella, Vibrio cholera, Coxiella burnetii, Francisella tularensis, Chlamydia psittaci, Ricinus communis, Rickettsia prowazekii, bacterial of the genus Salmonella (e.g., S. typhi), bacteria of the genus Shigella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis). In other embodiments, the composition comprising an immunogen comprises a viral pathogen or pathogen component including, but not limited to, influenza A virus, avian influenza virus, H₅N₁ influenza virus, West Nile virus, SARS virus, Marburg virus, Arenaviruses, Nipah virus, alphaviruses, filoviruses, herpes simplex virus I, herpes simplex virus II, sendai, sindbis, vaccinia, parvovirus, human immunodeficiency virus, hepatitis B virus, hepatitis C virus, hepatitis A virus, cytomegalovirus, human papilloma virus, picornavirus, hantavirus, junin virus, and ebola virus). In still further embodiments, the composition comprising an immunogen comprises a fungal pathogen or pathogen component, including, but not limited to, Candida albicnas and parapsilosis, Aspergillus fumigatus and niger, Fusarium spp, Trychophyton spp.

In some embodiments, a nanoemulsion adjuvant is administered to a subject before, concurrent with or after administration of a vaccine containing peptides (e.g., one generally well known in the art, as exemplified by U.S. Pat. Nos. 4,601,903; 4,599,231; 4,599,230; and 4,596,792; each of which is hereby incorporated by reference).

### Formulation Techniques

Nanoemulsions of the present invention can be formed using classic emulsion forming techniques. In brief, the oil phase is mixed with the aqueous phase under relatively high shear forces (e.g., using high hydraulic and mechanical forces) to obtain an oil-in-water nanoemulsion. The emulsion is formed by blending the oil phase with an aqueous phase on a volume-to-volume basis ranging from about 1:9 to 5:1, preferably about 5:1 to 3:1, most preferably 4:1, oil phase to aqueous phase. The oil and aqueous phases can be blended using any apparatus capable of producing shear forces sufficient to form an emulsion such as French Presses or high shear mixers (e.g., FDA approved high shear mixers are available, for example, from Admix, Inc., Manchester, N.H.). Methods of producing such emulsions are described in U.S. Pat. No. 5,103,497 and U.S. Pat. No. 4,895,452, and U.S. Patent Application Nos. 20070036831, 20060251684, and 20050208083, herein incorporated by reference in their entireties.

In preferred embodiments, compositions used in the methods of the present invention comprise droplets of an oily discontinuous phase dispersed in an aqueous continuous phase, such as water. In preferred embodiments, nanoemulsions of the present invention are stable, and do not decompose even after long storage periods (e.g., greater than one or more years). Furthermore, in some embodiments, nanoemulsions are stable (e.g., in some embodiments for greater than 3 months, in some embodiments for greater than 6 months, in some embodiments for greater than 12 months, in some embodiments for greater than 18 months) after combination with an immunogen. In preferred embodiments, nanoemulsions of the present invention are non-toxic and safe when administered (e.g., via spraying or contacting mucosal surfaces, swallowed, inhaled, etc.) to a subject.

In some embodiments, a portion of the emulsion may be in the form of lipid structures including, but not limited to, unilamellar, multilamellar, and paucliamellar lipid vesicles, micelles, and lamellar phases.

In general, the preferred non-toxic nanoemulsions are characterized by the following: they are approximately 200-800 nm in diameter, although both larger and smaller diameter nanoemulsions are contemplated; the charge depends on the ingredients; they are stable for relatively long periods of time (e.g., up to two years), with preservation of their biocidal activity; they are non-irritant and non-toxic compared to their individual components due, at least in part, to their oil contents that markedly reduce the toxicity of the detergents and the solvents; they are effective at concentrations as low as, for example, 0.1%; they have antimicrobial activity against most vegetative bacteria (including Gram-positive and Gram-negative organisms), fungi, and enveloped and nonenveloped viruses in 15 minutes (e.g., 99.99% killing); and they have sporicidal activity in 1-4 hours (e.g., 99.99% killing) when produced with germination enhancers.

The present invention is not limited by the type of subject administered a composition of the present invention. The present invention is not limited by the particular formulation of a composition comprising a nanoemulsion adjuvant of the present invention. Indeed, a composition comprising a nanoemulsion of the present invention may comprise one or more different agents in addition to the nanoemulsion. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion of the present invention comprises an agent and/or co-factor that enhance the ability of the nanoemulsion to induce an immune response. In some preferred embodiments, the presence of one or more co-factors or agents reduces the amount of nanoemulsion required for inducing an immune response. The present invention is not limited by the type of co-factor or agent used in a therapeutic agent of the present invention.

In some embodiments, a co-factor or agent used in a nanoemulsion composition is a bioactive agent. For example, in some embodiments, the bioactive agent may be a bioactive agent useful in a cell (e.g., a cell expressing a CFTR). Bioactive agents, as used herein, include diagnostic agents such as radioactive labels and fluorescent labels. Bioactive agents also include molecules affecting the metabolism of a cell (e.g., a cell expressing a CFTR), including peptides, nucleic acids, and other natural and synthetic drug molecules. Bioactive agents include, but are not limited to, adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunct to; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LHRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; amyotrophic lateral sclerosis agent; cerebral ischemia agent; Paget's disease agent; unstable angina agent; uricosuric; vasoconstrictor; vasodilator; vulnerary; wound healing agent; xanthine oxidase inhibitor.

Molecules useful as antimicrobials can be delivered by the methods and compositions of the invention. Antibiotics that may find use in co-administration with a composition comprising a nanoemulsion of the present invention include, but are not limited to, agents or drugs that are bactericidal and/or bacteriostatic (e.g., inhibiting replication of bacteria or inhibiting synthesis of bacterial components required for survival of the infecting organism), including, but not limited to, almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424.

In some embodiments, a composition comprising a nanoemulsion of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. Although an understanding of the mechanism is not necessary to practice the present invention and the present invention is not limited to any particular mechanism of action, in some embodiments, use of a mucoadhesive (e.g., in a composition comprising a nanoemulsion) enhances an immune response in a host subject due to an increase in duration and/or amount of exposure to the nanoemulsion that a subject experiences when a mucoadhesive is used compared to the duration and/or amount of exposure to the nanoemulsion in the absence of using the mucoadhesive.

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, pulmonary, subcutaneous, intramuscular, intraperitoneal, intravenous, or administration via other routes may be found in Remington's Pharmaceutical Sciences, Mack Publishing Company, Easton, Pa.

A composition comprising a nanoemulsion adjuvant of the present invention can be used therapeutically or as a prophylactic. A composition comprising a nanoemulsion of the present invention can be administered to a subject via a number of different delivery routes and methods (e.g., in a heterologous prime/boost regimen).

For example, the compositions of the present invention can be administered to a subject (e.g., mucosally or by pulmonary route) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal or pulmonary surface); being placed on or impregnated onto a nasal and/or pulmonary applicator and applied; being applied by a controlled-release mechanism; applied using a nebulizer, aerosolized, being applied as a liposome; or being applied on a polymer.

In some embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal and pulmonary techniques), as well as European Publication No. 517,565 and Illum et al., J. Controlled Rel., 1994, 29:133-141 (e.g., for techniques of intranasal administration), each of which is hereby incorporated by reference in its entirety). The present invention is not limited by the route of administration.

Methods of intranasal and pulmonary administration are well known in the art, including the administration of a droplet or spray form of the nanoemulsion into the nasopharynx of a subject to be treated. In some embodiments, a nebulized or aerosolized composition comprising a nanoemulsion is provided. Enteric formulations such as gastro resistant capsules for oral administration, suppositories for rectal or vaginal administration may also form part of this invention. Compositions of the present invention may also be administered via the oral route. Under these circumstances, a composition comprising a nanoemulsion may comprise a pharmaceutically acceptable excipient and/or include alkaline buffers, or enteric capsules. Formulations for nasal delivery may include those with dextran or cyclodextran and saponin as an adjuvant.

In some embodiments, a nanoemulsion of the present invention is administered via a pulmonary delivery route and/or means. In some embodiments, an aqueous solution containing the nanoemulsion is gently and thoroughly mixed to form a solution. The solution is sterile filtered (e.g., through a 0.2 micron filter) into a sterile, enclosed vessel. Under sterile conditions, the solution is passed through an appropriately small orifice to make droplets (e.g., between 0.1 and 10 microns).

The particles may be administered using any of a number of different applicators. Suitable methods for manufacture and administration are described in the following U.S. Pat. Nos. 6,592,904; 6,518,239; 6,423,344; 6,294,204; 6,051,256 and 5,997,848 to INHALE (now NEKTAR); and U.S. Pat. No. 5,985,309; RE37,053; U.S. Pat. Nos. 6,436,443; 6,447,753; 6,503,480; and 6,635,283, to Edwards, et al. (MIT, AIR), each of which is hereby incorporated

Thus, in some embodiments, compositions of the present invention are administered by pulmonary delivery. For example, a composition of the present invention can be delivered to the lungs of a subject (e.g., a human) via inhalation (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989; 84:1145-1146; Oswein, et al. “Aerosolization of Proteins”, 1990; Proceedings of Symposium on Respiratory Drug Delivery II Keystone, Colo.; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)). In some embodiments, a composition comprising a nanoemulsion is administered to a subject by more than one route or means (e.g., administered via pulmonary route as well as a mucosal route).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the ULTRAVENT nebulizer (Mallinckrodt Inc., St. Louis, Mo.); the ACORN II nebulizer (Marquest Medical Products, Englewood, Colo.); the VENTOLIN metered dose inhaler (Glaxo Inc., Research Triangle Park, N.C.); and the SPINHALER powder inhaler (Fisons Corp., Bedford, Mass.). All such devices require the use of formulations suitable for dispensing of therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising a nanoemulsion of the present invention may be used to protect and/or treat a subject susceptible to, or suffering from, a disease by means of administering (e.g., via a heterologous prime/boost administration protocol) compositions comprising a nanoemulsion by mucosal, intramuscular, intraperitoneal, intradermal, transdermal, pulmonary, intravenous, subcutaneous or other route of administration described herein. Methods of systemic administration of the nanoemulsion and/or agent co-administered with the nanoemulsion may include conventional syringes and needles, or devices designed for ballistic delivery (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). In some embodiments, the present invention provides a delivery device for systemic administration, pre-filled with the nanoemulsion composition of the present invention.

As described above, the present invention is not limited by the type of subject administered a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism. In some embodiments, the human subjects are subjects that are more likely to receive a direct exposure to pathogenic microorganisms or that are more likely to display signs and symptoms of disease after exposure to a pathogen (e.g., subjects with CF or asthma, subjects in the armed forces, government employees, frequent travelers, persons attending or working in a school or daycare, health care workers, an elderly person, an immunocompromised person, and emergency service employees (e.g., police, fire, EMT employees)). In some embodiments, any one or all members of the general public can be administered a composition of the present invention (e.g., to prevent the occurrence or spread of disease). For example, in some embodiments, compositions and methods of the present invention are utilized to treat a group of people (e.g., a population of a region, city, state and/or country) for their own health (e.g., to prevent or treat disease) and/or to prevent or reduce the risk of disease spread from animals (e.g., birds, cattle, sheep, pigs, etc.) to humans. In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition comprising a nanoemulsion of the present invention can be administered (e.g., to a subject (e.g., via a heterologous prime/boost administration protocol)) as a therapeutic or as a prophylactic to prevent microbial infection.

The compositions of the present invention may additionally contain other adjunct components conventionally found in pharmaceutical compositions. Thus, for example, the compositions may contain additional, compatible, pharmaceutically-active materials such as, for example, antipyruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the nanoemulsion. In some embodiments, nanoemulsion compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a nanoemulsion adjuvant is co-administered with one or more antibiotics. For example, one or more antibiotics may be administered with, before and/or after administration of a composition comprising a nanoemulsion. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

A wide variety of antimicrobial agents are currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a nanoemulsion adjuvant with one or more additional active and/or anti-infective agents. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, a second type of nanoemulsion, etc.) can be any of the well-known agents in the art, including, but not limited to, those that are currently in clinical use.

As described herein, in some embodiments, a composition comprising a nanoemulsion is administered to a subject via a heterologous prime/boost administration protocol. For example, a subject may benefit from receiving mucosal administration (e.g., nasal administration or other mucosal routes described herein) and, additionally, receiving one or more other routes of administration (e.g., injection (e.g., intramuscular injection) pulmonary administration (e.g., via a nebulizer, inhaler, or other methods described herein).

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

The present invention is not limited by the amount of nanoemulsion used. The amount will vary depending upon which specific nanoemulsion(s) is/are employed, and can vary from subject to subject, depending on a number of factors including, but not limited to, the species, age and general condition (e.g., health) of the subject, and the mode of administration. Procedures for determining the appropriate amount of nanoemulsion administered to a subject to induce an immune response in a subject can be readily determined using known means by one of ordinary skill in the art.

In some embodiments, it is expected that each dose (e.g., of a composition comprising a nanoemulsion comprises 1-40% nanoemulsion, in some embodiments, 20% nanoemulsion, in some embodiments less than 20% (e.g., 15%, 10%, 8%, 5% 4%, 3%, 2%, 1% or less nanoemulsion), and in some embodiments greater than 20% nanoemulsion (e.g., 25%, 30%, 35%, 40% or more nanoemulsion). An optimal amount for a particular administration can be ascertained by one of skill in the art using standard studies involving observation of immune responses described herein.

In some embodiments, it is expected that each dose (e.g., of a composition comprising a nanoemulsion is from 0.001 to 40% or more (e.g., 0.001-10%, 0.5-5%, 1-3%, 2%, 6%, 10%, 15%, 20%, 30%, 40% or more) by weight nanoemulsion.

Similarly, the present invention is not limited by the duration of time a nanoemulsion is administered to a subject. In some embodiments, a nanoemulsion is administered one or more times (e.g. twice, three times, four times or more) daily. In some embodiments, a composition comprising a nanoemulsion is administered one or more times a day until a suitable level of immune response is generated and/or the immune response is sustained. In some embodiments, a composition comprising a nanoemulsion of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of the nanoemulsion present in the concentrated composition. Concentrated compositions are contemplated to be useful in a setting in which large numbers of subjects may be administered a composition of the present invention (e.g., a hospital). In some embodiments, a composition comprising a nanoemulsion of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

Dosage units may be proportionately increased or decreased based on several factors including, but not limited to, the weight, age, and health status of the subject. In addition, dosage units may be increased or decreased for subsequent administrations.

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions, immunogens, and other components and for screening for new components. Thus, it is not intended that the present invention be limited to any particular subject and/or application setting.

The formulations can be tested in vivo in a number of animal models developed for the study of pulmonary, mucosal and other routes of delivery. As is readily apparent, the compositions of the present invention are useful for preventing and/or treating a wide variety of diseases and infections caused by viruses, bacteria, parasites, and fungi. Not only can the compositions be used prophylactically or therapeutically, as described above, the compositions can also be used in order to prepare antibodies, both polyclonal and monoclonal (e.g., for diagnostic purposes), as well as for immunopurification of an antigen of interest.

In one embodiment, the nanoemulsion compositions of the present invention are useful for the production of immunogenic compositions that can be used to generate antigen-specific antibodies that are useful in the specific identification of that antigen in an immunoassay according to a diagnostic embodiment. Such immunoassays include enzyme-linked immunosorbant assays (ELISA), RIAs and other non-enzyme linked antibody binding assays or procedures known in the art. In ELISA assays, the antigen-specific antibodies are immobilized onto a selected surface; for example, the wells of a polystyrene microtiter plate. After washing to remove incompletely adsorbed antibodies, a nonspecific protein, such as a solution of bovine serum albumin (BSA) or casein, that is known to be antigenically neutral with regard to the test sample may be bound to the selected surface. This allows for blocking of nonspecific adsorption sites on the immobilizing surface and thus reduces the background caused by nonspecific bindings of antigens onto the surface. The immobilizing surface is then contacted with a sample, such as clinical or biological materials, to be tested in a manner conducive to immune complex (antigen/antibody) formation. This may include diluting the sample with diluents, such as BSA, bovine gamma globulin (BGG) and/or phosphate buffered saline (PBS)/Tween. The sample is then allowed to incubate for from about 2 to 4 hours, at temperatures such as of the order of about 25-37° C. Following incubation, the sample-contacted surface is washed to remove non-immunocomplexed material. The washing procedure may include washing with a solution such as PBS/Tween, or a borate buffer.

Following formation of specific immunocomplexes between the antigen in the test sample and the bound antigen-specific antibodies, and subsequent washing, the occurrence, and even amount, of immunocomplex formation may be determined by subjecting the immunocomplex to a second antibody having specificity for the antigen. To provide detecting means, the second antibody may have an associated activity, such as an enzymatic activity, that will generate, for example, a color development upon incubating with an appropriate chromogenic substrate. Quantification may then achieved by measuring the degree of color generation using, for example, a visible spectra spectrophotometer. In an additional embodiment, the present invention includes a diagnostic kit comprising antigen-specific antibodies generated by immunization of a host with immunogenic compositions produced according to the present invention.

In some embodiments, the present invention provides a kit comprising a composition comprising a nanoemulsion adjuvant. In some embodiments, the kit further provides a device for administering the composition. The present invention is not limited by the type of device included in the kit. In some embodiments, the device is configured for pulmonary application of the composition of the present invention (e.g., a nasal inhaler or nasal mister). In some embodiments, a kit comprises a composition comprising a nanoemulsion in a concentrated form (e.g., that can be diluted prior to administration to a subject).

In some embodiments, all kit components are present within a single container (e.g., vial or tube). In some embodiments, each kit component is located in a single container (e.g., vial or tube (e.g., a nanoemulsion adjuvant is present in one container and an immunogen is present in a second, separate container)). In some embodiments, one or more kit components are located in a single container (e.g., vial or tube) with other components of the same kit being located in a separate container (e.g., vial or tube). In some embodiments, a kit comprises a buffer. In some embodiments, the kit further comprises instructions for use.

### Animal Models

In some embodiments, nanoemulsion adjuvant compositions (e.g., for generating an immune response (e.g., for use as an adjuvant and/or vaccine) are tested in animal models of infectious diseases. The use of well-developed animal models provides a method of measuring the effectiveness and safety of a vaccine before administration to human subjects. Exemplary animal models of disease are shown in Table 2. These animals are commercially available (e.g., from Jackson Laboratories Charles River; Portage, Mich.).

Animal models of Bacillus cereus (closely related to Bacillus anthracis) are utilized to test Anthrax vaccines of the present invention. Both bacteria are spore forming Gram positive rods and the disease syndrome produced by each bacteria is largely due to toxin production and the effects of these toxins on the infected host (Brown et al., J. Bact., 75:499 (1958); Burdon and Wende, J. Infect Dis., 107:224 (1960); Burdon et al., J. Infect. Dis., 117:307 (1967)). Bacillus cereus infection mimics the disease syndrome caused by Bacillus anthracis. Mice are reported to rapidly succumb to the effects of B. cereus toxin and are a useful model for acute infection. Guinea pigs develop a skin lesion subsequent to subcutaneous infection with B. cereus that resembles the cutaneous form of anthrax.

Clostridium perfringens infection in both mice and guinea pigs has been used as a model system for the in vivo testing of antibiotic drugs (Stevens et al., Antimicrob. Agents Chemother., 31:312 (1987); Stevens et al., J. Infect. Dis., 155:220 (1987); Alttemeier et al., Surgery, 28:621 (1950); Sandusky et al., Surgery, 28:632 (1950)). Clostridium tetani is well known to infect and cause disease in a variety of mammalian species. Mice, guinea pigs, and rabbits have all been used experimentally (Willis, Topley and Wilson's Principles of Bacteriology, Virology and Immunity. Wilson, G., A. Miles, and M. T. Parker, eds. pages 442-475 1983).

Vibrio cholerae infection has been successfully initiated in mice, guinea pigs, and rabbits. According to published reports it is preferred to alter the normal intestinal bacterial flora for the infection to be established in these experimental hosts. This is accomplished by administration of antibiotics to suppress the normal intestinal flora and, in some cases, withholding food from the animals (Butterton et al., Infect. Immun., 64:4373 (1996); Levine et al., Microbiol. Rev., 47:510 (1983); Finkelstein et al., J. Infect. Dis., 114:203 (1964); Freter, J. Exp. Med., 104:411 (1956); and Freter, J. Infect. Dis., 97:57 (1955)).

Shigella flexnerii infection has been successfully initiated in mice and guinea pigs. As is the case with vibrio infections, it is preferred that the normal intestinal bacterial flora be altered to aid in the establishment of infection in these experimental hosts. This is accomplished by administration of antibiotics to suppress the normal intestinal flora and, in some cases, withholding food from the animals (Levine et al., Microbiol. Rev., 47:510 (1983); Freter, J. Exp. Med., 104:411 (1956); Formal et al., J. Bact., 85:119 (1963); LaBrec et al., J. Bact. 88:1503 (1964); Takeuchi et al., Am. J. Pathol., 47:1011 (1965)).

Mice and rats have been used extensively in experimental studies with Salmonella typhimurium and Salmonella enteriditis (Naughton et al., J. Appl. Bact., 81:651 (1996); Carter and Collins, J. Exp. Med., 139:1189 (1974); Collins, Infect. Immun., 5:191 (1972); Collins and Carter, Infect. Immun., 6:451 (1972)).

Mice and rats are well established experimental models for infection with Sendai virus (Jacoby et al., Exp. Gerontol., 29:89 (1994); Massion et al., Am. J. Respir. Cell Mol. Biol. 9:361 (1993); Castleman et al., Am. J. Path., 129:277 (1987); Castleman, Am. J. Vet. Res., 44:1024 (1983); Mims and Murphy, Am. J. Path., 70:315 (1973)).

Sindbis virus infection of mice is usually accomplished by intracerebral inoculation of newborn mice. Alternatively, weanling mice are inoculated subcutaneously in the footpad (Johnson et al., J. Infect. Dis., 125:257 (1972); Johnson, Am. J. Path., 46:929 (1965)).

It is preferred that animals are housed for 3-5 days to rest from shipping and adapt to new housing environments before use in experiments. At the start of each experiment, control animals are sacrificed and tissue is harvested to establish baseline parameters. Animals are anesthetized by any suitable method (e.g., including, but not limited to, inhalation of Isofluorane for short procedures or ketamine/xylazine injection for longer procedure).

TABLE 2
Animal Models of Infectious Diseases

| | | Experimental | | | |
|---|---|---|---|---|---|
| | Experimental | Animal | | | Route of |
| Microorganism | Animal Species | Strains | Sex | Age | Infection |
| Francisella | mice | BALB/C | M | 6 W | Intraperitoneal |
| philomiraga | | | | | |
| Neisseria | mice | BALB/C | F | 6-10 W | Intraperitoneal |
| meningitidis | rats | COBS/CD | M/F | 4 D | Intranasal |
| Streptococcus | mice | BALB/C | F | 6 W | Intranasal |
| pneumoniae | rats | COBS/CD | M | 6-8 W | Intranasal |
| | guinea Pigs | Hartley | M/F | 4-5 W | Intranasal |
| Yersinia | mice | BALB/C | F | 6 W | Intranasal |
| pseudotuberculosis | | | | | |
| Influenza virus | mice | BALB/C | F | 6 W | Intranasal |
| Sendai virus | mice | CD-1 | F | 6 W | Intranasal |
| | rats | Sprague- | M | 6-8 W | Intranasal |
| | | Dawley | | | |
| Sindbis | mice | CD-1 | M/F | 1-2 D | Intracerebral/SC |
| Vaccinia | mice | BALB/C | F | 2-3 W | Intradermal |

E. Assays For Evaluation of Adjuvants and Vaccines

In some embodiments, nanoemulsion adjuvants and/or vaccines comprising the same are evaluated using one of several suitable model systems. For example, cell-mediated immune responses can be evaluated in vitro. In addition, an animal model may be used to evaluate in vivo immune response and immunity to pathogen challenge. Any suitable animal model may be utilized, including, but not limited to, those disclosed in Table 2.

Before testing a nanoemulsion vaccine in an animal system, the amount of exposure of the pathogen to a nanoemulsion sufficient to inactivate the pathogen is investigated. It is contemplated that pathogens such as bacterial spores require longer periods of time for inactivation by the nanoemulsion in order to be sufficiently neutralized to allow for immunization. The time period required for inactivation may be investigated using any suitable method, including, but not limited to, those described in the illustrative examples below.

In addition, the stability of emulsion-developed vaccines is evaluated, particularly over time and storage condition, to ensure that vaccines are effective long-term. The ability of other stabilizing materials (e.g., dendritic polymers) to enhance the stability and immunogenicity of vaccines is also evaluated.

Once a given nanoemulsion/pathogen vaccine has been formulated to result in pathogen inactivation, the ability of the vaccine to elicit an immune response and provide immunity is optimized. Non-limiting examples of methods for assaying vaccine effectiveness are described in Example 14 below. For example, the timing and dosage of the vaccine can be varied and the most effective dosage and administration schedule determined. The level of immune response is quantitated by measuring serum antibody levels. In addition, in vitro assays are used to monitor proliferation activity by measuring H³-thymidine uptake. In addition to proliferation, Th1 and Th2 cytokine responses (e.g., including but not limited to, levels of include IL-2, TNF-γ, IFN-γ, IL-4, IL-6, IL-11, IL-12, etc.) are measured to qualitatively evaluate the immune response.

Finally, animal models are utilized to evaluate the effect of a nanoemulsion mucosal vaccine. Purified pathogens are mixed in emulsions (or emulsions are contact with a pre-infected animal), administered, and the immune response is determined. The level of protection is then evaluated by challenging the animal with the specific pathogen and subsequently evaluating the level of disease symptoms. The level of immunity is measured over time to determine the necessity and spacing of booster immunizations.

### III. Therapeutics and Prophylactics

Furthermore, in preferred embodiments, a nanoemulsion adjuvant composition of the present invention induces (e.g., when administered to a subject) innate and adaptive/acquired immune responses (e.g., both systemic and mucosal immunity). Thus, in some preferred embodiments, administration of a composition of the present invention to a subject results in protection against an exposure (e.g., a mucosal exposure) to a pathogen. Although an understanding of the mechanism is not necessary to practice the present invention and the present invention is not limited to any particular mechanism of action, mucosal administration (e.g., vaccination) provides protection against pathogen infection (e.g., that initiates at a mucosal surface). Although it has heretofore proven difficult to stimulate secretory IgA responses and protection against pathogens that invade at mucosal surfaces (See, e.g., Mestecky et al, Mucosal Immunology. 3ed edn. (Academic Press, San Diego, 2005)), the present invention provides compositions and methods for stimulating mucosal immunity (e.g., a protective IgA response) from a pathogen in a subject.

In some embodiments, the present invention provides a composition (e.g., a composition comprising a NE and immunogenic protein antigens (e.g., from a pathogen (e.g., gp120)) to serve as a mucosal vaccine. This material can easily be produced with NE and pathogen derived protein (e.g., recombinantly produced or viral-derived gp120, live-virus-vector-derived gp120 and gp160, recombinant mammalian gp120, recombinant denatured antigens, small peptide segments of gp120 and gp41, V3 loop peptides), and induces both mucosal and systemic immunity. The ability to produce this formulation rapidly and administer it via mucosal (e.g., nasal or vaginal) instillation provides a vaccine that can be used in large-scale administrations (e.g., to a population of a town, village, city, state or country).

In some preferred embodiments, the present invention provides a composition for generating an immune response comprising a NE and an immunogen (e.g., a purified, isolated or synthetic protein or derivative, variant, or analogue thereof; or, one or more serotypes of pathogens inactivated by the nanoemulsion). When administered to a subject, a composition of the present invention stimulates an immune response against the immunogen/pathogen within the subject. Although an understanding of the mechanism is not necessary to practice the present invention and the present invention is not limited to any particular mechanism of action, in some embodiments, generation of an immune response (e.g., resulting from administration of a composition comprising a nanoemulsion and an immunogen) stimulates innate and/or adaptive/acquired immune responses that provides total or partial immunity to the subject (e.g., from signs, symptoms or conditions of a disease (e.g., caused by the pathogen)). Without being bound to any specific theory, protection and/or immunity from disease (e.g., the ability of a subject's immune system to prevent or attenuate (e.g., suppress) a sign, symptom or condition of disease) after exposure to an immunogenic composition of the present invention is due to adaptive (e.g., acquired) immune responses (e.g., immune responses mediated by B and T cells following exposure to a NE comprising an immunogen of the present invention (e.g., immune responses that exhibit increased specificity and reactivity towards the pathogen). Thus, in some embodiments, the compositions and methods of the present invention are used prophylactically or therapeutically to prevent or attenuate a sign, symptom or condition associated with the pathogen.

In some embodiments, a nanoemulsion adjuvant is administered alone. In some embodiments, a nanoemulsion adjuvant comprises one or more other agents (e.g., a pharmaceutically acceptable carrier, other adjuvant, excipient, and the like). In some embodiments, a nanoemulsion adjuvant is administered in a manner to induce a humoral immune response. In some embodiments, a nanoemulsion adjuvant is administered in a manner to induce a cellular (e.g., cytotoxic T lymphocyte) immune response, rather than a humoral response. In some embodiments, a nanoemulsion adjuvant induces both a cellular and humoral immune response.

The present invention is not limited by the particular formulation of a composition comprising a nanoemulsion adjuvant (e.g., independently or together with an immunogen) of the present invention. Indeed, a composition comprising a nanoemulsion adjuvant of the present invention may comprise one or more different agents in addition to the nanoemulsion adjuvant. These agents or cofactors include, but are not limited to, additional adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion adjuvant of the present invention comprises an agent and/or co-factor that enhance the ability of the nanoemulsion adjuvant to induce an immune response. In some preferred embodiments, the presence of one or more co-factors or agents reduces the amount of nanoemulsion adjuvant required for induction of an immune response (e.g., a protective immune response (e.g., protective immunization)). In some embodiments, the presence of one or more co-factors or agents can be used to skew the immune response towards a cellular (e.g., T cell mediated) or humoral (e.g., antibody mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995. The present invention is not limited by the type of adjuvant utilized (e.g., for use in a composition (e.g., pharmaceutical composition) comprising a nanoemulsion adjuvant). For example, in some embodiments, suitable adjuvants include an aluminium salt such as aluminium hydroxide gel (alum) or aluminium phosphate. In some embodiments, an adjuvant may be a salt of calcium, iron or zinc, or may be an insoluble suspension of acylated tyrosine, or acylated sugars, cationically or anionically derivatised polysaccharides, or polyphosphazenes.

In some embodiments, a composition comprising a nanoemulsion adjuvant described herein (e.g., with or without an immunogen) comprises one or more additional adjuvants that induce and/or skew toward a Th1-type response. However, in other embodiments, it will be preferred that a composition comprising a nanoemulsion adjuvant described herein (e.g., with or without an immunogen) comprises one or more additional adjuvants that induce and/or skew toward a Th2-type response.

In general, an immune response is generated to an antigen through the interaction of the antigen with the cells of the immune system. Immune responses may be broadly categorized into two categories: humoral and cell mediated immune responses (e.g., traditionally characterized by antibody and cellular effector mechanisms of protection, respectively). These categories of response have been termed Th1-type responses (cell-mediated response), and Th2-type immune responses (humoral response).

Stimulation of an immune response can result from a direct or indirect response of a cell or component of the immune system to an intervention (e.g., exposure to an immunogen). Immune responses can be measured in many ways including activation, proliferation or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titer; splenomegaly (including increased spleen cellularity); hyperplasia and mixed cellular infiltrates in various organs. Other responses, cells, and components of the immune system that can be assessed with respect to immune stimulation are known in the art.

Although an understanding of the mechanism is not necessary to practice the present invention and the present invention is not limited to any particular mechanism of action, in some embodiments, compositions and methods of the present invention induce expression and secretion of cytokines (e.g., by macrophages, dendritic cells and CD4+ T cells (See, e.g., Example 8). Modulation of expression of a particular cytokine can occur locally or systemically. It is known that cytokine profiles can determine T cell regulatory and effector functions in immune responses. In some embodiments, Th1-type cytokines can be induced, and thus, the immunostimulatory compositions of the present invention can promote a Th1 type antigen-specific immune response including cytotoxic T-cells. However in other embodiments, Th2-type cytokines can be induced thereby promoting a Th2 type antigen-specific immune response.

Cytokines play a role in directing the T cell response. Helper (CD4+) T cells orchestrate the immune response of mammals through production of soluble factors that act on other immune system cells, including B and other T cells. Most mature CD4+ T helper cells express one of two cytokine profiles: Th1 or Th2. Th1-type CD4+ T cells secrete IL-2, IL-3, IFN-γ, GM-CSF and high levels of TNF-α. Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities whereas those associated with Th2 responses are associated more with allergic responses.

Several factors have been shown to influence skewing of an immune response towards either a Th1 or Th2 type response. The best characterized regulators are cytokines IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production.

Thus, in some preferred embodiments, the present invention provides a method of stimulating a Th1-type immune response in a subject comprising administering to a subject a composition comprising a nanoemulsion adjuvant described herein (e.g., with or without an immunogen). However, in other preferred embodiments, the present invention provides a method of stimulating a Th2-type immune response in a subject comprising administering to a subject a composition comprising a nanoemulsion adjuvant described herein (e.g., with or without an immunogen). In further preferred embodiments, additional adjuvants can be used (e.g., can be co-administered with a nanoemulsion adjuvant composition of the present invention) to skew an immune response toward either a Th1 or Th2 type immune response. For example, adjuvants that induce Th2 or weak Th1 responses include, but are not limited to, alum, saponins, and SB-As4. Adjuvants that induce Th1 responses include but are not limited to MPL, MDP, ISCOMS, IL-12, IFN-γ, and SB-AS2.

Several other types of Th1-type immunogens can be used (e.g., as an adjuvant) in compositions and methods of the present invention. These include, but are not limited to, the following. In some embodiments, monophosphoryl lipid A (e.g., in particular 3-de-O-acylated monophosphoryl lipid A (3D-MPL)), is used. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. In some embodiments, diphosphoryl lipid A, and 3-O-deacylated variants thereof are used. Each of these immunogens can be purified and prepared by methods described in GB 2122204B, hereby incorporated by reference in its entirety. Other purified and synthetic lipopolysaccharides have been described (See, e.g., U.S. Pat. No. 6,005,099 and EP 0 729 473; Hilgers et al., 1986, Int. Arch. Allergy. Immunol., 79(4):392-6; Hilgers et al., 1987, Immunology, 60(1):141-6; and EP 0 549 074, each of which is hereby incorporated by reference in its entirety). In some embodiments, 3D-MPL is used in the form of a particulate formulation (e.g., having a small particle size less than 0.2 μm in diameter, described in EP 0 689 454, hereby incorporated by reference in its entirety).

In some embodiments, saponins are used as an immunogen (e.g., Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit Rev Ther Drug Carrier Syst, 1996, 12 (1-2):1-55; and EP 0 362 279, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146, 431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety.

In some embodiments, an immunogenic oligonucleotide containing unmethylated CpG dinucleotides (“CpG”) is used as an adjuvant in the present invention. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Immunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

Although an understanding of the mechanism is not necessary to practice the present invention and the present invention is not limited to any particular mechanism of action, in some embodiments, the presence of one or more CpG oligonucleotides activate various immune subsets including natural killer cells (which produce IFN-γ) and macrophages. In some embodiments, CpG oligonucleotides are formulated into a composition of the present invention for inducing an immune response. In some embodiments, a free solution of CpG is co-administered together with an antigen (e.g., present within a NE solution (See, e.g., WO 96/02555; hereby incorporated by reference). In some embodiments, a CpG oligonucleotide is covalently conjugated to an antigen (See, e.g., WO 98/16247, hereby incorporated by reference), or formulated with a carrier such as aluminium hydroxide (See, e.g., Brazolot-Millan et al., Proc. Natl. AcadSci., USA, 1998, 95(26), 15553-8).

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. Coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (See, e.g., WO93/13202 and WO92/19265, each of which is hereby incorporated by reference), and other immunogenic substances (e.g., that enhance the effectiveness of a composition of the present invention) are used with a composition comprising a NE and immunogen of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants may be added to a composition comprising a nanoemulsion adjuvant and an immunogen, or, the adjuvant may be formulated with carriers, for example liposomes, or metallic salts (e.g., aluminium salts (e.g., aluminium hydroxide)) prior to combining with or co-administration with a composition comprising a nanoemulsion adjuvant and an immunogen.

In some embodiments, a composition comprising a nanoemulsion adjuvant and an immunogen comprises a single additional adjuvant. In other embodiments, a composition comprising a nanoemulsion adjuvant and an immunogen comprises two or more additional adjuvants (See, e.g., WO 94/00153; WO 95/17210; WO 96/33739; WO 98/56414; WO 99/12565; WO 99/11241; and WO 94/00153, each of which is hereby incorporated by reference in its entirety).

In some embodiments, a composition comprising a NE adjuvant described herein (e.g., with or without an immunogen) of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. In some embodiments, one or more components of the NE adjuvant function as a mucoadhesive (e.g., individually, or in combination with other components of the NE adjuvant). Although an understanding of the mechanism is not necessary to practice the present invention and the present invention is not limited to any particular mechanism of action, in some embodiments, use of a mucoadhesive (e.g., in a composition comprising a NE and immunogen) enhances induction of an immune response (e.g., an innate and/or adaptive immune response) in a subject (e.g., a subject administered a composition of the present invention) due to an increase in duration and/or amount of exposure to NE adjuvant and/or immunogen that a subject experiences when a mucoadhesive is used compared to the duration and/or amount of exposure to an immunogen in the absence of using the mucoadhesive).

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising a nanoemulsion adjuvant and an immunogen of the present invention can be used therapeutically (e.g., to enhance an immune response) or as a prophylactic (e.g., for immunization (e.g., to prevent signs or symptoms of disease)). A composition comprising a nanoemulsion adjuvant and an immunogen of the present invention can be administered to a subject via a number of different delivery routes and methods.

For example, the compositions of the present invention can be administered to a subject (e.g., mucosally (e.g., nasal mucosa, genital mucosa, oral mucosa, rectal mucosa, etc.)) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal surface); being placed on or impregnated onto a nasal and/or vaginal applicator and applied; being applied by a controlled-release mechanism; being applied as a liposome; or being applied on a polymer.

In some preferred embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal and rectal techniques), as well as European Publication No. 517,565 and Illum et al., J. Controlled Rel., 1994, 29:133-141 (e.g., for techniques of intranasal administration), each of which is hereby incorporated by reference in its entirety). Alternatively, the compositions of the present invention may be administered dermally or transdermally, using standard techniques (See, e.g., Remington: The Science arid Practice of Pharmacy, Mack Publishing Company, Easton, Pa., 19th edition, 1995). The present invention is not limited by the route of administration.

Although an understanding of the mechanism is not necessary to practice the present invention and the present invention is not limited to any particular mechanism of action, in some embodiments, mucosal vaccination is the preferred route of administration (e.g., for one of the routes of administration chosen for heterologous prime/boost administration) as it has been shown that mucosal administration of antigens has a greater efficacy of inducing protective immune responses at mucosal surfaces (e.g., mucosal immunity), the route of entry of many pathogens. In addition, mucosal vaccination, such as intranasal vaccination, may induce mucosal immunity not only in the nasal mucosa, but also in distant mucosal sites such as the genital mucosa (See, e.g., Mestecky, Journal of Clinical Immunology, 7:265-276, 1987). More advantageously, in further preferred embodiments, in addition to inducing mucosal immune responses, mucosal vaccination also induces systemic immunity.

In some embodiments, a composition comprising a nanoemulsion adjuvant and an immunogen of the present invention may be used to protect or treat a subject susceptible to, or suffering from, disease by means of administering a composition of the present invention via a mucosal route (e.g., an oral/alimentary or nasal route). Alternative mucosal routes include intravaginal and intra-rectal routes. In preferred embodiments of the present invention, a nasal route of administration is used, termed “intranasal administration” or “intranasal vaccination” herein. Methods of intranasal vaccination are well known in the art, including the administration of a droplet or spray form of the vaccine into the nasopharynx of a subject to be immunized. In some embodiments, a nebulized or aerosolized composition comprising a nanoemulsion adjuvant and immunogen is provided. Enteric formulations such as gastro resistant capsules for oral administration, suppositories for rectal or vaginal administration also form part of this invention. Compositions of the present invention may also be administered via the oral route. Under these circumstances, a composition comprising a nanoemulsion adjuvant and an immunogen may comprise a pharmaceutically acceptable excipient and/or include alkaline buffers, or enteric capsules. Formulations for nasal delivery may include those with dextran or cyclodextran and saponin as an adjuvant.

Compositions of the present invention may also be administered via a vaginal route. In such cases, a composition comprising a nanoemulsion adjuvant and an immunogen may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, a composition comprising a NE and an immunogen may comprise excipients and/or waxes and polymers known in the art for forming rectal suppositories.

In some embodiments, the same route of administration (e.g., mucosal administration) is chosen for both a priming and boosting vaccination. In some embodiments, multiple routes of administration are utilized (e.g., at the same time, or, alternatively, sequentially (e.g., in a heterologous prime/boost administration protocol) in order to stimulate an immune response (e.g., using a composition comprising a nanoemulsion adjuvant and immunogen of the present invention).

For example, in some embodiments, a composition comprising a nanoemulsion adjuvant and an immunogen is administered to a mucosal surface of a subject in either a priming or boosting vaccination regime. Alternatively, in some embodiments, a composition comprising a nanoemulsion adjuvant and an immunogen is administered systemically in either a priming or boosting vaccination regime. In some embodiments, a composition comprising a nanoemulsion adjuvant and an immunogen is administered to a subject in a priming vaccination regimen via mucosal administration and a boosting regimen via a different route of administration (e.g., injection (e.g., intramuscular injection)). In some embodiments, a composition comprising a nanoemulsion adjuvant and an immunogen is administered to a subject in a priming vaccination regimen via a non-mucosal route (e.g., injection (e.g., intramuscular injection)) and a boosting regimen via mucosal administration. In some embodiments, a composition comprising a nanoemulsion adjuvant and an immunogen is administered to a subject in a priming vaccination regimen via mucosal administration and a boosting regimen via a systemic route. In some embodiments, a composition comprising a nanoemulsion adjuvant and an immunogen is administered to a subject in a priming vaccination regimen via a systemic route and a boosting regimen via mucosal administration. Examples of systemic routes of administration include, but are not limited to, a parenteral, intramuscular, intradermal, transdermal, subcutaneous, intraperitoneal or intravenous administration. A composition comprising a NE and an immunogen may be used for both prophylactic and therapeutic purposes.

In some embodiments, compositions of the present invention are administered by pulmonary delivery. For example, a composition of the present invention can be delivered to the lungs of a subject (e.g., a human) via inhalation (e.g., thereby traversing across the lung epithelial lining to the blood stream (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989; 84:1145-1146; Oswein, et al. “Aerosolization of Proteins”, 1990; Proceedings of Symposium on Respiratory Drug Delivery II Keystone, Colo.; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for dispensing of therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising a nanoemulsion adjuvant of the present invention may be used to protect and/or treat a subject susceptible to, or suffering from, a disease by means of administering a compositions comprising a nanoemulsion adjuvant by mucosal, intramuscular, intraperitoneal, intradermal, transdermal, pulmonary, intravenous, subcutaneous or other route of administration described herein. Methods of systemic administration of the adjuvant preparations may include conventional syringes and needles, or devices designed for ballistic delivery of solid vaccines (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). The present invention may also be used to enhance the immunogenicity of antigens applied to the skin (transdermal or transcutaneous delivery, See, e.g., WO 98/20734; WO 98/28037, each of which are hereby incorporated by reference). Thus, in some embodiments, the present invention provides a delivery device for systemic administration, pre-filled with the adjuvant composition of the present invention.

The present invention is not limited by the type of subject administered (e.g., in order to stimulate an immune response (e.g., in order to generate protective immunity (e.g., mucosal and/or systemic immunity))) a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism. In some embodiments, the human subjects are subjects that are more likely to receive a direct exposure to pathogenic microorganisms or that are more likely to display signs and symptoms of disease after exposure to a pathogen (e.g., immune suppressed subjects). In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease). In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition of the present invention may be formulated for administration by any route, such as mucosal, oral, topical, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxyethyl) pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments, pharmaceutical compositions of the present invention may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the nanoemulsion adjuvant and immunogen of the formulation. In some embodiments, immunostimulatory compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a nanoemulsion adjuvant is co-administered with one or more antibiotics. For example, one or more antibiotics may be administered with, before and/or after administration of a composition comprising a nanoemulsion adjuvant. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

There are an enormous amount of antimicrobial agents currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a nanoemulsion adjuvant with one or more additional active and/or immunostimulatory agents. Indeed, it is a further aspect of this invention to provide methods for enhancing prior art immunostimulatory methods (e.g., immunization methods) and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, adjuvants, etc.) can be any of the well-known agents in the art, including, but not limited to, those that are currently in clinical use.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In preferred embodiments, a composition comprising a nanoemulsion adjuvant and an immunogen of the present invention comprises a suitable amount of the immunogen to induce an immune response in a subject when administered to the subject. In preferred embodiments, the immune response is sufficient to provide the subject protection (e.g., immune protection) against a subsequent exposure to the immunogen or the microorganism (e.g., bacteria or virus) from which the immunogen was derived. The present invention is not limited by the amount of immunogen used. In some preferred embodiments, the amount of immunogen (e.g., virus or bacteria neutralized by the nanoemulsion adjuvant, or, recombinant protein) in a composition comprising a nanoemulsion adjuvant and immunogen (e.g., for use as an immunization dose) is selected as that amount which induces an immunoprotective response without significant, adverse side effects. The amount will vary depending upon which specific immunogen or combination thereof is/are employed, and can vary from subject to subject, depending on a number of factors including, but not limited to, the species, age and general condition (e.g., health) of the subject, and the mode of administration. Procedures for determining the appropriate amount of immunogen administered to a subject to elicit an immune response (e.g., a protective immune response (e.g., protective immunity)) in a subject are well known to those skilled in the art.

In some embodiments, it is expected that each dose (e.g., of a composition comprising a nanoemulsion adjuvant and an immunogen (e.g., administered to a subject to induce an immune response (e.g., a protective immune response (e.g., protective immunity))) comprises 0.05-5000 μg of each immunogen (e.g., recombinant and/or purified protein), in some embodiments, each dose will comprise 1-500 μg, in some embodiments, each dose will comprise 350-750 μg, in some embodiments, each dose will comprise 50-200 μg, in some embodiments, each dose will comprise 25-75 μg of immunogen (e.g., recombinant and/or purified protein). In some embodiments, each dose comprises an amount of the immunogen sufficient to generate an immune response. An effective amount of the immunogen in a dose need not be quantified, as long as the amount of immunogen generates an immune response in a subject when administered to the subject. An optimal amount for a particular administration (e.g., to induce an immune response (e.g., a protective immune response (e.g., protective immunity))) can be ascertained by one of skill in the art using standard studies involving observation of antibody titers and other responses in subjects.

In some embodiments, it is expected that each dose (e.g., of a composition comprising a nanoemulsion adjuvant and an immunogen (e.g., administered to a subject to induce and immune response)) is from 0.001 to 15% or more (e.g., 0.001-10%, 0.5-5%, 1-3%, 2%, 6%, 10%, 15% or more) by weight immunogen (e.g., neutralized bacteria or virus, or recombinant and/or purified protein). In some embodiments, an initial or prime administration dose contains more immunogen than a subsequent boost dose

In some embodiments, a composition comprising a nanoemulsion adjuvant of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising about 0.1-50% of the nanoemulsion adjuvant present in the concentrated composition. In some preferred embodiments, a subject is administered in a single dose a composition comprising 1% of the NE and immunogen present in the concentrated composition. Concentrated compositions are contemplated to be useful in a setting in which large numbers of subjects may be administered a composition of the present invention (e.g., an immunization clinic, hospital, school, etc.). In some embodiments, a composition comprising a nanoemulsion adjuvant of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

Generally, the emulsion compositions of the invention will comprise at least 0.001% to 100%, preferably 0.01 to 90%, of emulsion per ml of liquid composition. It is envisioned that the formulations may comprise about 0.001%, about 0.0025%, about 0.005%, about 0.0075%, about 0.01%, about 0.025%, about 0.05%, about 0.075%, about 0.1%, about 0.25%, about 0.5%, about 1.0%, about 2.5%, about 5%, about 7.5%, about 10%, about 12.5%, about 15%, about 20%, about 25%, about 30%, about 35%, about 40%, about 50%, about 55%, about 60%, about 65%, about 70%, about 75%, about 80%, about 85%, about 90%, about 95% or about 100% of emulsion per ml of liquid composition. It should be understood that a range between any two figures listed above is specifically contemplated to be encompassed within the metes and bounds of the present invention. Some variation in dosage will necessarily occur depending on the condition of the specific pathogen and the subject being immunized.

In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. Although an understanding of the mechanism is not necessary to practice the present invention and the present invention is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

A composition comprising an immunogen of the present invention finds use where the nature of the infectious and/or disease causing agent (e.g., for which protective immunity is sought to be elicited) is known, as well as where the nature of the infectious and/or disease causing agent is unknown (e.g., in emerging disease (e.g., of pandemic proportion (e.g., influenza or other outbreaks of disease))). For example, the present invention contemplates use of the compositions of the present invention in treatment of or prevention of infections associated with an emergent infectious and/or disease causing agent yet to be identified (e.g., isolated and/or cultured from a diseased person but without genetic, biochemical or other characterization of the infectious and/or disease causing agent).

### EXAMPLES

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); μmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar); ° C. (degrees Centigrade); and PBS (phosphate buffered saline).

### Example 1

### NE Formulation and Route of Administration can Influence Type of Immune Response

Experiments were conducted during development of embodiments of the invention in order to determine if a heterologous prime/boost administration regimen would affect immune responses generated in subjects. In particular, experiments were conducted during development of embodiments of the invention in order to determine if combined, heterologous intranasal and intramuscular administration of an immunogenic composition (comprising nanoemulsion plus antigen) would alter immune response generated (e.g., improve activation of Th-1 type response induced by an immunogenic composition comprising nanoemulsion/immunogen.

Study Design: C57BL/6 mice: 5 per group were administered the following nanoemulsion plus antigen: 20 μg HBsAg. For intranasal administration, the immunogenic composition contained a NE concentration of 20% and 20 μg HBsAg, with a total volume of 15 μl administered. For intramuscular administration, the immunogenic composition contained a NE concentration of 5% and 20 μg HBsAg, with a total volume of 50 μl administered. The immunogenic composition utilize for administration via each route of the heterologous prime/boost administration routes contained the same nanoemulsion, at different concentrations. As shown in Table 3, prime administration took place at Week 0, with Boost at Week 3. Serum antibody was obtained at 2-3 week intervals (0, 2, 5 weeks). Cellular immune responses were evaluated at sacrifice (2 weeks after boost). Table 3 shows the administration protocol used:

TABLE 3
Administration of nanoemulsion plus antigen: 20 μg HBsAg, for IN
administration, a total volume of 15 μl of NE plus antigen was
administered, the NE concentration was 20%; for IM administration,
a total volume of 50 μl of NE plus antigen was administered, the NE
concentration was 5%.

| VACCINE | PRIME | BOOST |
|---|---|---|
| NE-HBsAg | IN | IN |
| | IM | IM |
| | IN | IM |
| PBS-HBsAg | IN | IM |
| | IM | IM |

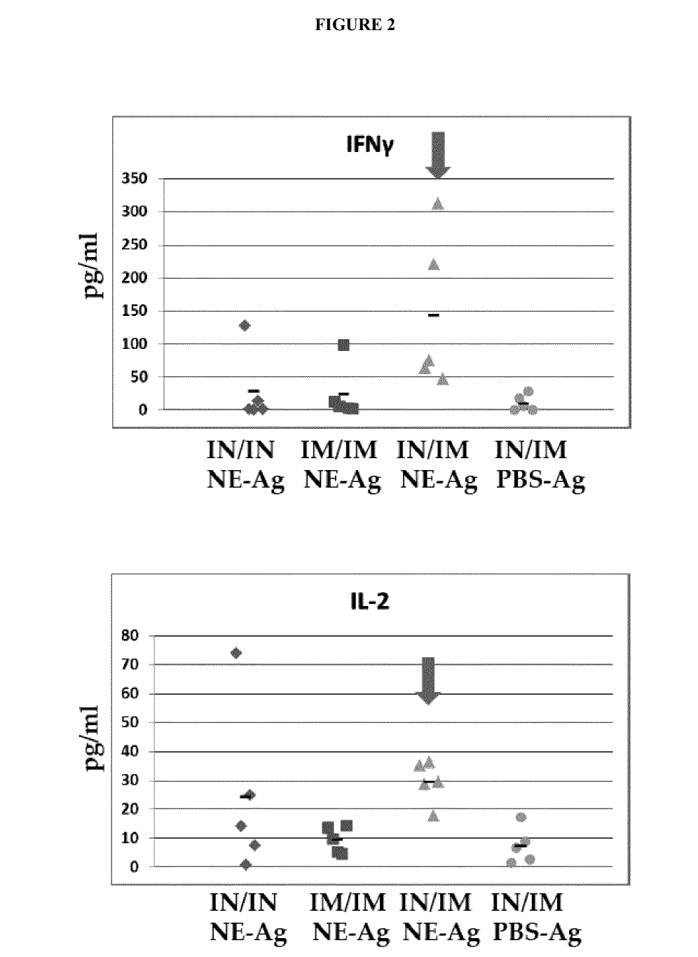

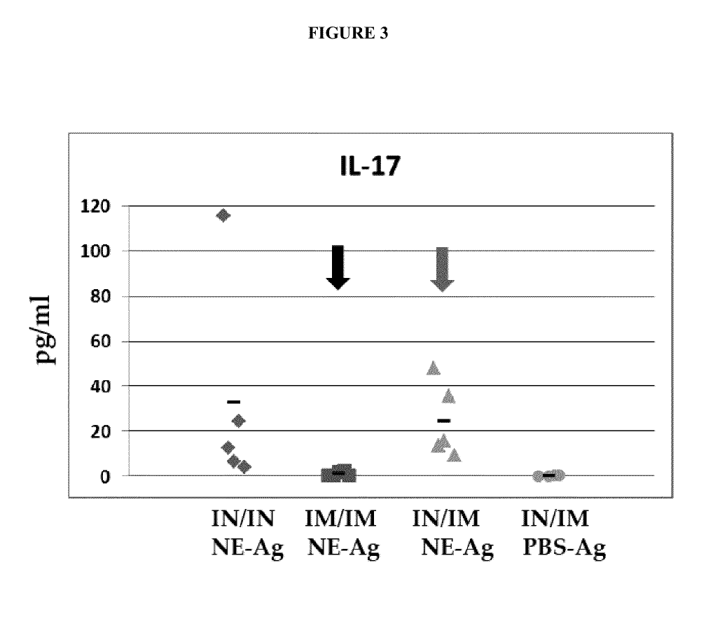

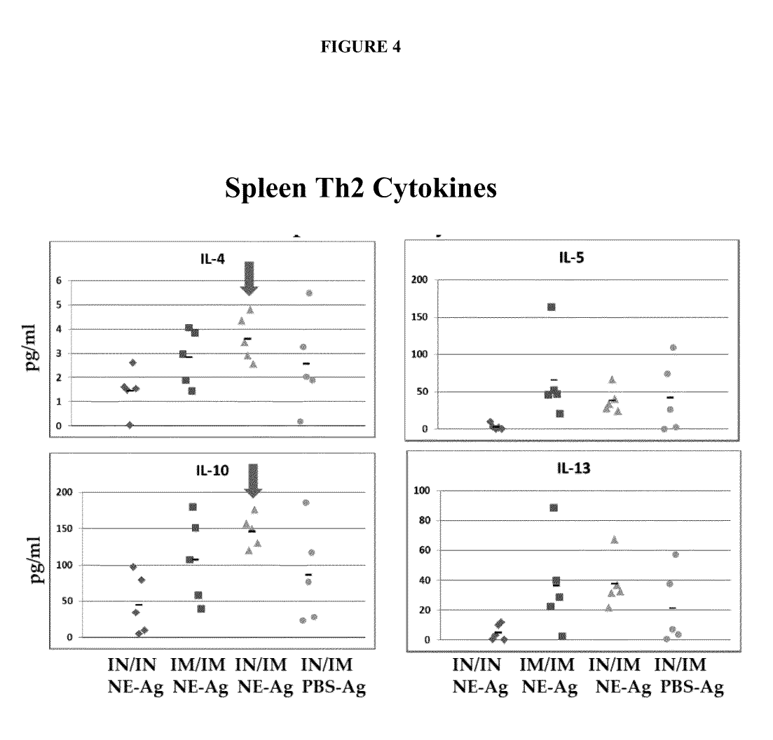

Experiments performed indicated that the route of NE administration drives the type of immune response when an immunogenic composition comprising nanoemulsion and respiratory syncytial virus (NE-RSV) was administered (See FIG. 1). The heterologous prime/boost administration protocol enhanced production of Th1-type cytokines in response to HBsAg (FIG. 2). There was also a strong Th17 response via intranasal but not intramuscular route, and the IN/IM heterologous prime/boost strategy maintained Th17 type immune response (FIG. 3). The heterologous prime/boost administration regimen enhanced production of Th2-type cytokines (FIG. 4). In particular, IM route activated higher Th2 responses compared to IN alone. Heterologous administration enhanced production of Th2 cytokines compared to IN alone (IL-4,5,10,13) or IM alone (IL-4,10) (See FIG. 4).

The heterologous prime/boost administration protocol also enhanced anti-HBsAg serum IgG response compared to IN route alone (See FIG. 5). IM route rapidly activated IgG response compared to IN route at 2 weeks. IM administration activated higher IgG response compared to IN route after boost at 5 weeks. The heterologous prime/boost administration protocol enhanced IgG response compared to IN route alone at 5 weeks (See FIG. 5). Accordingly, in some embodiments, the invention provides that NE exhibits strong adjuvant effect via a heterologous prime/boost administration protocol (IN, IM and heterologous routes). Moreover, the invention provides that the same nanoemulsion formulation, delivered via different routes, effectively induces robust immune responses.

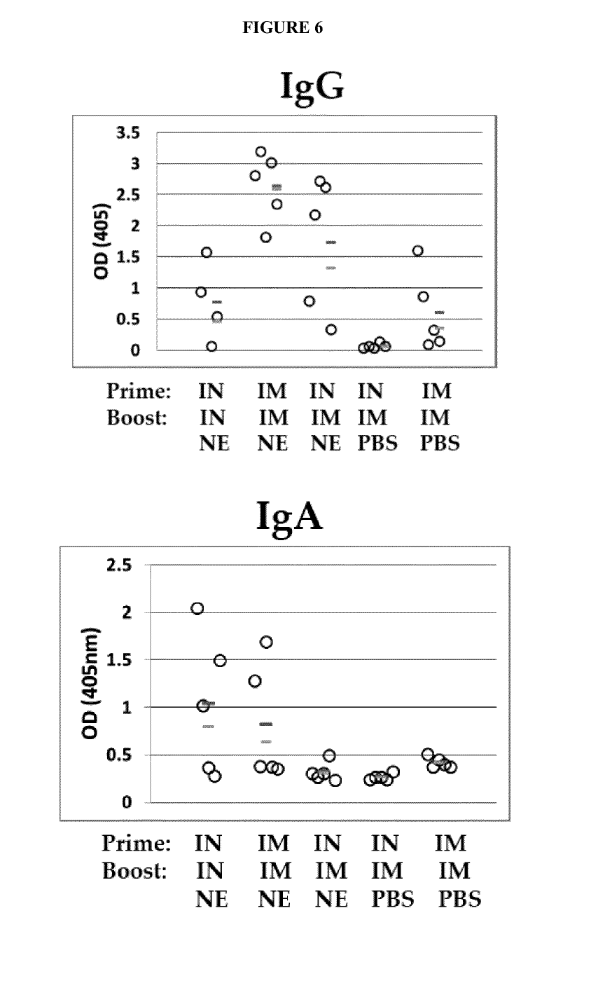

The heterologous prime/boost administration protocol also enhanced anti-HBsAg-specific IgG antibody responses in Bronchial Alveolar Lavage (BAL) compared to IN route alone (See FIG. 6). In particular, whereas IM administration activated the highest IgG response in BAL, IN administration activated the highest IgA response in BAL. The heterologous prime/boost administration (IN/IM route) enhanced IgG but not IgA response compared to IN route alone. Thus, the invention provides, in some embodiments, that a heterologous prime/boost administration protocol is useful to induce and maintain a Th17 response; increase Th1 (e.g., IFNγ) and Th2 (e.g., IL-4, IL-10) responses compared to either route alone; and/or enhance antigen specific total IgG in serum and BAL. The invention also provides that a heterologous prime/boost administration protocol is useful to reduce subject to subject immune response variation.

### Example 2

### Levels of Anti-F IgG in Sera of Cotton Rats 2 Weeks after 3rd Immunization or 4 Weeks after 1 Dose of IM Immunization

During the development of embodiments of the inventions provided herein, experiments were conducted to evaluate the immunogenic capacity of NE-antigen compositions administered IN and IM to a model mammalian system (e.g., a rat). In particular, animals were immunized three times with immunogenic compositions comprising nanoemulsion and RSV antigen (NE-RSV). The NE-RSV compositions were administered IN and IM and sera were obtained to evaluate the presence of antibodies against an antigen of RSV, e.g., the RSV fusion (F) protein. Formalin inactivated RSV (FI-RSV) and RSV strain A2 (A2 infection) were used as controls. After drawing sera, IgG antibodies against RSV fusion (F) protein were quantified (see FIG. 7).

As shown in FIG. 7, all groups generated significant antibody levels. NE-RSV yielded the lowest levels of serum antibodies (e.g., relative to the same dose administered IM). In addition, one IM immunization yielded a significantly higher level of antibodies than three IN immunizations. Results are recorded as the geometric mean±95% confidence limits (GM±95% C1).

### Example 3

### Neutralization Activity in Sera of Cotton Rats 2 Weeks after 3rd Immunization with IN Versus IM Vaccine

During the development of embodiments of the inventions provided herein, experiments were performed to evaluate the neutralization of live virus by antibodies induced by NE-RSV administered IN and IM. Neutralization assays were performed in Vero cell culture. Plates are inoculated with Vero cells and RSV virus is added in the presence of increasing dilutions of the serum being tested for neutralizing activity. Virus added with non-immune serum was used as a positive control. The serum dilution that results in a 50% reduction of the virus titer is measured and the values reported as the inverse of the dilution that resulted in 50% inhibition of the viral infection (e.g., a serum sample that produced a 50% inhibition at a dilution of 1:250 has a neutralization activity (NU) of 250 units.

After immunization of Cotton rats three times with NE-RSV administered IN or IM, sera were drawn and evaluated for neutralization of live RSV in Vero cell culture (see FIG. 8). Sera drawn from rats administered FI-RSV and RSV strain A2 were used as controls. The relative activities of the sera to neutralize live virus was similar to the relative antibody titers measured in Example 2 (e.g., compare FIG. 8 with FIG. 7). In particular, the data collected from testing the immune sera show that the neutralizing activity of serum from IN immunization is significantly lower than the neutralizing activity of sera drawn after IM administration or infection (FIG. 8).

However, the serum antibodies generated by IM or IN administration or by infection had similar neutralizing activity (FIG. 9). That is, the specific activities (e.g., neutralization activity against live virus per unit weight of antibodies) of antibodies produced by IN administration of NE-RSV, IM administration of NE-RSV, and infection with RSV strain A2 were similar (FIG. 9). These data show that the antibodies generated by IN and IM administration of NE-antigen have the same functional activities. As shown by the data (FIG. 9), only the FI-RSV vaccine produced a defective immune response characterized by a low specific activity compared to the other vaccines.

### Example 4

### Viral Clearance in the Lungs of Cotton Rats Administered Vaccine IN and IM

During the development of embodiments of the inventions provided herein, experiments were conducted to test the clearance of RSV from the lungs by immunized rats. In particular, Cotton rats were immunized with NE-RSV administered IN and NE-RSV administered IM. Rats administered a vaccine comprising FI-RSV and naive rats were used as controls. After immunization, rats were challenged with a live RSV infection. The data collected showed that the rats immunized with NE-RSV administered IN, NE-RSV administered IM, and FI-RSV cleared the subsequent viral challenge completely (e.g., below the limit of detection (LOD) of 5×10¹ plaque forming units (PFU) per gram).

### Example 5

### Heterologous Immunization with RSV Versus 1 Dose IM

During the development of embodiments of the inventions described herein, experiments were conducted to test antibody generation in animals immunized according to a heterologous prime/boost administration protocol comprising both IN and IM adiministration of NE-RSV. In this study, animals were primed at time zero by immunization with NE-RSV via the IN route, NE-RSV via the IM route, or by infection with live RSV strain A. After a wait period of up to 12 weeks (e.g., to establish immunological memory), animals were boosted via immunization with NE-RSV via the IN route or NE-RSV via the IM route. Animals were bled 2 weeks later for evaluation.

As shown in FIG. 11, animals of the first group (“IM/none/IN”) were immunized IM on day zero and after 12 weeks these animals were administered a booster immunization IN. Animals of the second group (“IN/none/IM”) were immunized IN on day zero and after 12 weeks these animals were administered a booster immunization IM. Similarly, animals (groups 3 (“Infection/none/IN”) and 4 (“Infection/none/IM”)) were infected with RSV strain A2 at time zero and allowed to recover for 12 weeks followed by booster administration via IN (group 3) or via IM (group 4). The last group (group 5 (“NE-RSV IM 4 weeks after 1 dose”)) was naïve animals that received one IM immunization and then were bled 4 weeks later to assess whether the memory afforded by IN immunization or by infection had any effect on the response to the subsequent IM immunization.

After immunization, IgG antibodies were quantified. Animals primed by infection or IN immunization did not support a booster response by subsequent IM immunization (FIG. 11). All groups primed or naive generated the same levels of antibodies after an IM immunization. IM immunization primed only for an IM boost (see FIG. 7).

### Example 6

### HSV-2 Prophylaxis Vaccine in Guinea Pig Animal Model

During the development of embodiments of the inventions described herein, experiments were conducted to test protection against infection by herpes simplex virus II (HSV-2) by IN and IM administration of a vaccine in guinea pig. Guinea pigs were immunized with a composition comprising a W85EC nanoemulsion and recombinant glycoprotein D2 (gD2) from HSV-2. A 20-μg dose was used in these formulations via mixing the antigen with the appropriate amount of nanoemulsion. The IN compositions comprised a 20% nanoemulsion concentration and the IM compositions comprised a 5% nanoemulsion. Sera from animals were obtained for quantification of IgG titers by ELISA and to assess functional (e.g., neutralization) activity. The neutralization assay and calculation of NU is identical to that described above except that HSV-2 is used in these experiments. After immunization, animals were challenged with 5×10⁵ plaque forming units (pfu) of virus intravaginally and the animals were observed for 13 days for appearance of HSV ulcers on the vaginal parts. The vaginal infection and/or ulceration was scored and the cumulative score is plotted against the non-immune animals.

Data collected show that animals immunized via the IM route produced significantly higher levels of antibodies compared to antibodies produced by IN administration. In addition, data showed that the functional (e.g., specific) activities and protection provided by the antibodies generated by IN and IM administration were the same. These data are similar to the results of the experiments described above for RSV in Example 3.

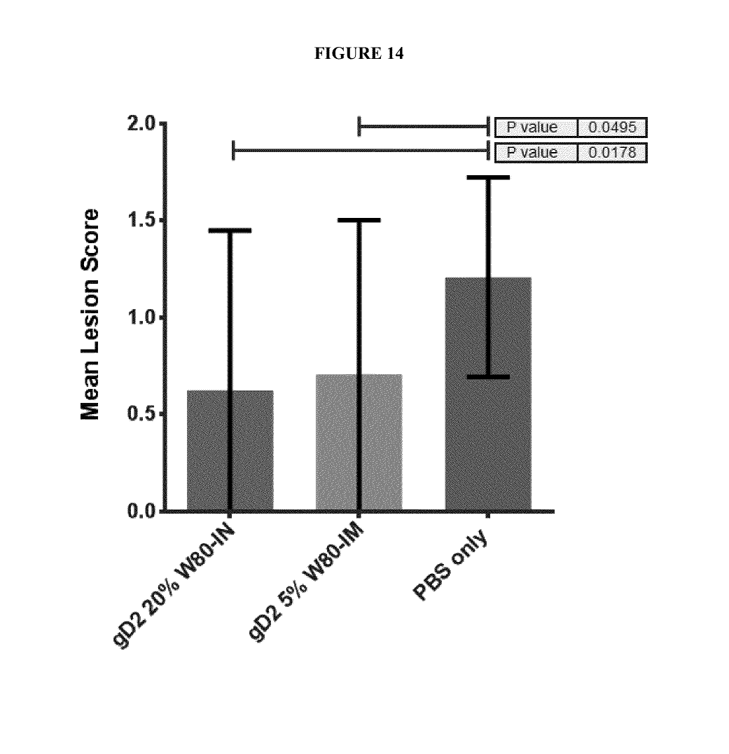

Experiments were conducted to test administration of HSV-2 vaccine via IN and IM routes in the guinea pig model. In particular, neutralization titers were assessed at week 11 after IN and IM immunization with a composition comprising nanoemulsion and the gD2 subunit of HSV-2 as antigen. A phosphate-buffered saline solution was used as a control. IM administration produced significantly higher levels of antibodies compared to IN administration (FIG. 12). Protection conferred by IN and IM administration of vaccines against vaginal challenge by HSV-2 infection were similar despite the differences in antibody titer and neutralization activities. As shown in FIG. 13, the cumulative scores for vaginal lesions on day 13 after the HSV-2 challenge were significantly lower for the IN and IM vaccinated animals compared to control. Protection against recurrence after the infection acute phase was also similar. As shown in FIG. 14, 33 days after challenge with HSV-2 infection, the mean lesion scores for the IN and IM vaccinated animals were lower than the control.

These data demonstrate that IN immunization resulted in lower serum antibodies and lower neutralization activity compared to the IM group, but still significantly higher than the PBS control group. Further, despite the highly significant (p=0.004) difference between the IM and the IN neutralization activities, both showed a significant protection against the viral challenge. These data suggest that the two routes of immunization operate by different modes. In particular, while IN immunization was shown to produce serum antibodies (e.g., at a titer lower than IM immunization), IN immunization also confers additional protection via a different mechanism than IM immunization, e.g., such as producing different T-cell mediated immunity cytokine biomarkers and a Th17 immune response (see FIGS. 1-6).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. A method for inducing a multi-component immunogen-specific immune response in a subject, the method comprising: administering to the subject an immunogenic composition comprising a nanoemulsion and an immunogen via a first route to induce a first component of an immunogen-specific immune response and administering to the subject an immunogenic composition comprising a nanoemulsion and an immunogen via a second route to induce a second component of an immunogen-specific immune response.

2. The method of claim 1, wherein the immunogenic composition is administered via a mucosal route of administration.

3. The method of claim 2, wherein the mucosal route of administration is via the nasal mucosa.

4. The method of claim 1, wherein the immunogenic composition is administered via a parenteral route of administration.

5. The method of claim 4, wherein the parenteral route of administration is selected from the group consisting of infusion, injection, and implantation.

6. The method of claim 5, wherein the injection is selected from the group consisting of subcutaneous injection, intramuscular injection, intradermal injection, intraperitoneal injection, and intravenous injection.

7. The method of claim 1, wherein the first component of the immunogen-specific immune response is not attainable by administering to the subject an immunogenic composition comprising a nanoemulsion and an immunogen via the second route alone.

8. The method of claim 1, wherein the second component of the immunogen-specific immune response is not attainable by administering to the subject an immunogenic composition comprising a nanoemulsion and an immunogen via the first route alone.

9. The method of claim 1 wherein the multi-component immunogen-specific immune response is not attainable by administering to the subject an immunogenic composition comprising a nanoemulsion and an immunogen via the first route alone.

10. The method of claim 1 wherein the multi-component immunogen-specific immune response is not attainable by administering to the subject an immunogenic composition comprising a nanoemulsion and an immunogen via the second route alone.

11. The method of claim 1, wherein the first route is a mucosal route and the second route is an intramuscular route.

12. The method of claim 1, wherein the same immunogenic composition is used for administering via the first route and for administering via the second route.

13. The method of claim 1, wherein the immunogenic composition administered via the first route and the immunogenic composition administered via the second route comprise:
a) the same immunogen and the same nanoemulsion; and
b) the same amount of immunogen; but
c) the percent of nanoemulsion present in the immunogenic composition administered via the first route is different than the percent of nanoemulsion present in the immunogenic composition administered via the second route.

14. The method of claim 1, wherein the amount of immunogen present in the immunogenic composition administered via the first route is the same as the amount of immunogen present in the immunogenic composition administered via the second route.

15. The method of claim 1, wherein the first component of the immunogen-specific immune response comprises induction of antibodies, induction of cytokines, and/or a T cell response and the second component of the immunogen-specific immune response comprises a different induction of antibodies, a different induction of cytokines, and/or a different T cell response.

16. The method of claim 1, wherein the first component of the immunogen-specific immune response comprises a Th17 type immune response.

17. The method of claim 1, wherein the second component of the immunogen-specific immune response comprises an increased titer of IgG antibodies.

18. The method of claim 1, wherein the second component of the immunogen-specific immune response comprises an increased titer of IgG antibodies that is 10 times to 100 times the titer of IgG antibodies of the first component of the immunogen-specific immune response.

19. The method of claim 1 further comprising one or both of administering to the subject a boost immunogenic composition comprising a nanoemulsion and an immunogen via the first route and/or administering to the subject a boost immunogenic composition comprising a nanoemulsion and an immunogen via the second route.

20. An immunization regimen for inducing a multi-component immunogen-specific immune response in a subject comprising (a) an immunogenic composition comprising a nanoemulsion and an immunogen for administration via a first route to induce a first component of an immunogen-specific immune response and (b) an immunogenic composition comprising a nanoemulsion and an immunogen for administration via a second route to induce a second component of an immunogen-specific immune response and comprising the same nanoemulsion as in (a).

21. The immunization regimen according to claim 20, wherein the same immunogen is present in both the immunogenic composition for administration via the first route and the immunogenic composition for administration via the second route.

22. The immunization regimen according to claim 20, wherein the same immunogen is present in the same quantity in both the immunogenic composition for administration via the first route and the immunogenic composition for administration via the second route.

23. The immunization regimen according to claim 20, wherein the first route is a mucosal route.

24. The immunization regimen according to claim 23, wherein the mucosal route is via nasal mucosa.

25. The immunization regimen according to claim 20, wherein the second route is a parenteral route.

26. The immunization regimen according to claim 20, wherein the first route is a mucosal route and wherein the second route is an intramuscular injection.

27. The immunization regimen according to claim 20, wherein the immunogenic composition for administration via the first route is the same as the immunogenic composition for administration via the second route.

28. The immunization regimen according to claim 20, wherein the immunogenic composition administered via the first route and the immunogenic composition administered via the second route comprise:
a) the same immunogen and the same nanoemulsion;
b) the same amount of immunogen; but
c) the percent of nanoemulsion present in the immunogenic composition administered via the first route is different than the percent of nanoemulsion present in the immunogenic composition administered via the second route.

29. The immunization regimen according to claim 20, wherein the immunogen present in the immunogenic composition administered via the first route is different than the immunogen present in the immunogenic composition administered via the second route.

30. The immunization regimen according to claim 20, wherein the immunogenic composition for administration via the first route and the immunogenic composition for administration via the second route further comprise an adjuvant.

31. The immunization regimen according to claim 20, wherein the immunogen is a cancer antigen.

32. The immunization regimen according to claim 20, wherein the immunogen is a viral immunogen.

33. The immunization regimen according to claim 32, wherein the viral antigen is a respiratory syncytial virus (RSV) antigen.

34. The immunization regimen according to claim 32, wherein the viral antigen is a herpes simplex virus (HSV) antigen

35. The immunization regimen according to claim 32, wherein the viral antigen is an influenza antigen.

36. The immunization regimen according to claim 20, wherein the immunogen is a bacterial antigen.

37. The immunization regimen according to claim 20, wherein the immunogen is a recombinant antigenic peptide.

38. The immunization regimen according to claim 37, wherein the peptide is a glycoprotein D2 subunit of HSV.
